(19) United States
(12) Patent Application Publication
(10) Pub. No.: US 2013/0053382 A1
(43) Pub. Date: Feb. 28, 2013

(54) Inhibitors of Phosphoinositide Dependent Kinase 1 (PDK1)

(71) Applicants: Sunil Paliwal, Monroe Township, NJ (US); Matthew Paul Rainka, Schenectady, NY (US); Ang Li, Cranberry Township, PA (US); May Xiaowu Jiang, Guilderland, NY (US); Hon-chung Tsui, Shanghai (CN); Ronald J. Doll, Convent Station, NJ (US)

(72) Inventors: Sunil Paliwal, Monroe Township, NJ (US); Matthew Paul Rainka, Schenectady, NY (US); Ang Li, Cranberry Township, PA (US); May Xiaowu Jiang, Guilderland, NY (US); Hon-chung Tsui, Shanghai (CN); Ronald J. Doll, Convent Station, NJ (US)

(21) Appl. No.: 13/643,580

(22) PCT Filed: Apr. 28, 2011
(86) PCT No.: PCT/US11/34275

§ 371 Date: Oct. 26, 2012

Related U.S. Application Data
(60) Provisional application No. 61/329,699, filed on Apr. 30, 2010.

(51) Int. Cl. C07D 417/14; A61K 31/5377; A61P 35/00; A61K 31/4725; A61K 31/454; A61K 31/4439; A61K 31/427; A61K 31/496

(52) U.S. Cl. 514/234.5; 548/200; 514/365; 544/133; 544/369; 514/254.02; 546/146; 514/307; 546/199; 514/322; 546/269.7; 514/338

(57) ABSTRACT

The instant invention provides for compounds that inhibit PDK1 activity. The invention also provides for compositions comprising such inhibitory compounds and methods of inhibiting PDK1 activity by administering the compound to a patient in need of treatment of cancer.

### BACKGROUND OF THE INVENTION

3-Phosphoinositide-dependent protein kinase-1 (PDK1) is a 556-amino acid containing enzyme comprised of a C-terminal Pleckstrin homology (PH) domain (residues 459-550) and an N-terminal kinase domain (residues 70-359). The PH domain of PDK1 binds phosphatidylinositols (e.g., phosphatidylinositol 4,5-bisphosphate and phosphatidylinositol 3,4,5-triphosphate) produced by phosphatidylinositol kinases, such as phosphatidylinositol 3-kinase (PI3K) and whose levels are, in part, controlled by phosphatases such as PTEN (phosphatase and tensin homologue). PDK1 plays a central role in the PI3K/Akt pathway and has been called a “master regulator” kinase due to its role as a critical upstream activating kinase that phosphorylates the so-called T-loop phosphorylation site for multiple kinases in the AGC family of kinases including but not limited to all three isoforms of PKB (PKBα, PKBβ, PKBγ, also known as Akt1, Akt2, and Akt3, respectively), RSK (three isoforms RSK1, RSK2, RSK3, also known as p90RSK), p70S6K (two isoforms, S6K1 and S6K2), SGK1 and PKC.

Signals from several peptide growth factors including insulin, insulin-like growth factor-1 and platelet-derived growth factor are transduced by PKB. Like PDK1, PKB contains a PH domain that binds phosphatidyl 3,4,5-triphosphate. PKB is translocated to the plasma membrane and phosphorylated by PDK1 at residue T-308/309 (the two phosphosites correspond to different isoforms) in response to the second messenger phosphatidyl 3,4,5-triphosphate produced by PI3K. Activation of PKB in tumor cells results in increased cellular survival via anti-apoptotic signals and also proliferation. PKBβ amplification has been observed in a proportion of several tumor types including ovarian, breast and pancreatic cancers. Similarly, PKBα amplification has been observed in a percentage of gastric adenocarcinoma samples. Recently, an activated mutant form of PKBα (E17K) was detected in a number of breast (8%), colorectal (6%), and ovarian (2%) cancers. PDK1 kinase inhibitors are useful as treatments for diseases linked to PKB signaling (such as cancer, Cowden syndrome, Lhermitte-Dudos disease and Bannayan-Zonana syndrome) by preventing activation of PKB signaling by PDK1.

Similarly, PDK1 kinase inhibitors are useful for treating cancer or other proliferative disorders by blocking the activation of p70S6K by PDK1. There are several substrates of p70S6K, such as ribosomal S6 protein, eIF4B, PDCD4 etc., that are involved in translation inhibition complex formation or ribosomal protein synthesis. Inhibition of protein synthesis via inhibition of phosphorylation of ribosomal S6 protein is believed to inhibit the proliferation of tumor cells by mTOR inhibitors (e.g., rapamycin). p70S6K gene amplification has been observed in breast tumor specimens. Simultaneous amplification of p70S6K and HER-2 correlates with poor survival in cancer patients. Hyperactivation of p70S6K (as measured by phosphorylation of T389) has been observed by immunohistochemical analysis of breast, head and neck squamous cell carcinoma (HNSCC), glioblastoma, lung and liver primary tumor specimens.

Likewise, PDK1 kinase inhibitors are useful for the treatment of cancer by blocking the activation of RSK1 (also known as p90RSK) by PDK1. RSK1 transduces anti-apoptotic and proliferative signals be mediating phosphorylation directly or indirectly of BAD, LKB1, TSC2, NFkB, mTOR. Ras/MAPK pathway is activated in >50% in primary tumors. RSK1 activity is correlated with MAPK activity. RSK1 is overexpressed in primary breast and prostate cancer samples.

PDK1 signaling regulates multiple critical steps in angiogenesis. Inhibitors of the activity of PDK1 are thus useful in the treatment of cancer, in particular cancers associated with deregulated activity of the PTEN/PI3K pathway including, but not limited to PTEN loss of function mutations, PI3K gain of function mutations and receptor tyrosine kinase gain of function mutations.

PDK1 signaling has also been implicated in tumorigenesis and a PDK1 inhibitor is useful for tumor prevention or prevention of tumor recurrence. Mice with a PTEN heterozygous (PTEN^{+/-}) genotype are well-known to spontaneously develop tumors. Alessi and co-workers found that PDK1 hypomorphic PTEN^{+/-} mice expressing <30% of normal PDK1 protein levels showed a significant delay in tumor formation as compared to littermate controls expressing normal levels of PDK1 protein (Current Biology, 2005, 15, 1839-1846).

SGK1 (serum and glucocorticoid-regulated kinase-1) activity is critical for insulin-mediated Na+ retention and hypertensive effects. Inhibition of SGK1 activity by a PDK1 kinase inhibitor is useful in treating hypertension and/or hypoinsulinemia.

It is an object of the instant invention to provide novel compounds that are inhibitors of PDK1.

It is also an object of the present invention to provide pharmaceutical compositions that comprise the novel compounds that are inhibitors of PDK1.

It is also an object of the present invention to provide a method for treating cancer that comprises administering such inhibitors of PDK1 activity.

It is also an object of the present invention to provide a method for treating tumor recurrence that comprises administering such inhibitors of PDK1 activity.

It is also an object of the present invention to provide a method for treating hypertension that comprises administering such inhibitors of PDK1 activity.

It is also an object of the present invention to provide a method for treating diabetes mellitus that comprises administering such inhibitors of PDK1 activity.

### SUMMARY OF THE INVENTION

The instant invention provides for compounds that inhibit PDK1 activity. The invention also provides for compositions comprising such inhibitory compounds and methods of inhibiting PDK1 activity by administering the compound to a patient in need of treatment of cancer.

### DETAILED DESCRIPTION OF THE INVENTION

The compounds of the instant invention are useful in the inhibition of the activity of PDK1. In a first embodiment of this invention, the inhibitors of PDK1 activity are illustrated by the Formula A:

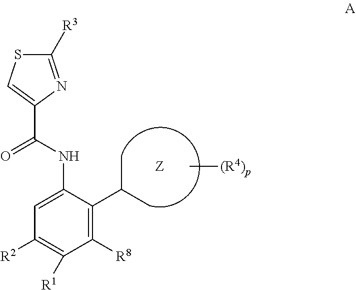

wherein:

a is 0 or 1, b is 0 or 1, m is 0, 1 or 2, and p is 0, 1, 2, 3 or 4;

ring Z is attached to phenyl via a carbon-carbon bond and is selected from cycloalkyl, cycloalkenyl, aryl, heteroaryl and heterocyclyl;

R¹ and R² are independently selected from CF₃, CN, halo, OH, C₁₋₆ alkyl and C₃₋₈ cycloalkyl, or R¹ and R² can be taken together to form a heterocyclyl which is optionally substituted with one to four substituents selected from CF₃, halo, N(R^{b})₂, OH, (O)C₁₋₃ alkyl, C₁₋₃ alkyl and C₃₋₆ cycloalkyl;

R³ is selected from hydrogen, CF₃, CN, halo, CO₂H, C₁₋₆ alkyl, C₂₋₆ alkenyl, C₂₋₆ alkynyl, C₃₋₈ cycloalkyl, C₃₋₈ cycloalkenyl, aryl, heteroaryl, heterocyclyl, N(R^{a})₂, C(═O)N(R^{a})₂, S(O)ₘ C₁₋₆ alkyl, S(O)ₘ C₃₋₈ cycloalkyl, S(O)ₘ C₃₋₈ cycloalkenyl, S(O)ₘ aryl, S(O)ₘ heteroaryl, S(O)ₘN(R^{a})₂, wherein said alkyl, alkenyl, alkynyl, cycloalkyl, cycloalkenyl, aryl, heteroaryl and heterocyclyl are optionally substituted with one to four substituents selected from CF₃, halo, OH, (O)C₁₋₆ alkyl, C₁₋₆ alkyl, C₃₋₆ cycloalkyl, aryl, heteroaryl, heterocyclyl, N(R^{a})₂, C(═O)N(R^{a})₂, S(O)ₘ C₁₋₆ alkyl, S(O)ₘ C₃₋₈ cycloalkyl, S(O)ₘ aryl, S(O)ₘ heteroaryl, S(O)ₘ N(R^{a})₂, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, aryl and heterocyclyl;

R⁴ is independently selected from CF₃, CN, halo, CO₂H, OH, C₁₋₆ alkyl, S(O)ₘ C₁₋₆ alkyl, N(R^{a})₂, C(═O)N(R^{a})₂, S(O)ₘN(R^{a})₂, C₃₋₈ cycloalkyl, C₃₋₈ cycloalkenyl, aryl, heteroaryl and heterocyclyl, wherein said alkyl, cycloalkyl, cycloalkenyl, aryl, heteroaryl and heterocyclyl are optionally substituted with one to four substituents selected from CF₃, halo, N(R^{a})₂, C(═O)N(R^{a})₂, OH, (O)C₁₋₆ alkyl, C₁₋₆ alkyl, C₃₋₆ cycloalkyl, aryl, heteroaryl, and heterocyclyl, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl and C₃₋₆ cycloalkyl;

R⁸ is hydrogen or halo;

R^{a} is independently selected from hydrogen, C₁₋₃ alkyl and C₃₋₆ cycloalkyl, wherein said alkyl and cycloalkyl are optionally substituted with CF₃, halo, N(R^{b})₂, OH, (O)C₁₋₃ alkyl, C₁₋₃ alkyl, C₃₋₆ cycloalkyl and phenyl; and

R^{b} is independently selected from hydrogen and C₁₋₃ alkyl;

or a pharmaceutically acceptable salt, tautomer or a stereoisomer thereof.

In a second embodiment of Formula A, the inhibitors of PDK1 activity are illustrated, wherein:

ring Z is attached to phenyl via a carbon-carbon bond and is selected from cyclohexenyl, pyridyl, isoindolinyl, isoquinolinyl, indenyl and phenyl;

R¹ and R² are independently selected from CF₃, CN, halo, OH, C₁₋₆ alkyl and C₃₋₈ cycloalkyl, or R¹ and R² can be taken together to form an imidazole, pyrrole, pyrazole, thiophene or furan;

R³ is pyrazolyl and C₂₋₆ alkenyl which are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl; and

all other substituents are as defined in the first embodiment of Formula A;

or a pharmaceutically acceptable salt, tautomer or a stereoisomer thereof.

In a third embodiment of Formula A, the inhibitors of PKD1 activity are illustrated, wherein,

R³ is pyrazolyl which is optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl; and

all other substituents are as defined in the second embodiment of Formula A;

or a pharmaceutically acceptable salt, tautomer or a stereoisomer thereof.

In a fourth embodiment of Formula A, the inhibitors of PKD1 activity are illustrated, wherein,

R³ is pyrazolyl which is optionally substituted with CF₃, C₁₋₃ alkyl, phenyl and heteroaryl, wherein said alkyl is optionally substituted with phenyl or heterocyclyl; and

all other substituents are as defined in the third embodiment of Formula A;

or a pharmaceutically acceptable salt, tautomer or a stereoisomer thereof.

In a fifth embodiment of Formula A, the inhibitors of PKD1 activity are illustrated, wherein,

R³ is pyrazolyl which is optionally substituted with CF₃; and

all other substituents are as defined in the fourth embodiment of Formula A;

or a pharmaceutically acceptable salt, tautomer or a stereoisomer thereof.

In a second embodiment of this invention, the inhibitors of PDK1 activity are illustrated by the Formula B:

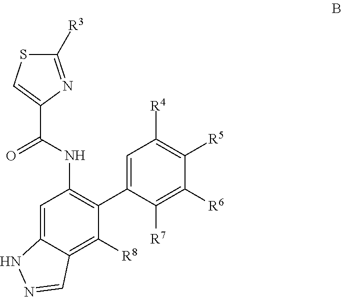

wherein:

a is 0 or 1, b is 0 or 1, and m is 0, 1 or 2;

R³ is selected from hydrogen, CF₃, CN, halo, CO₂H, C₁₋₆ alkyl, C₂₋₆ alkenyl, C₂₋₆ alkynyl, C₃₋₈ cycloalkyl, C₃₋₈ cycloalkenyl, aryl, heteroaryl, heterocyclyl, N(R^{a})₂, C(═O)N(R^{a})₂, S(O)ₘ C₁₋₆ alkyl, S(O)ₘ C₃₋₈ cycloalkyl, S(O)ₘ C₃₋₈ cycloalkenyl, S(O)ₘ aryl, S(O)ₘ heteroaryl, S(O)ₘ N(R^{a})₂, wherein said alkyl, alkenyl, alkynyl, cycloalkyl, cycloalkenyl, aryl, heteroaryl and heterocyclyl are optionally substituted with one to four substituents selected from CF₃, halo, OH, (O)C₁₋₆ alkyl, C₁₋₆ alkyl, C₃₋₆ cycloalkyl, aryl, heteroaryl, heterocyclyl, N(R^{a})₂, C(═O)N(R^{a})₂, S(O)ₘ C₁₋₆ alkyl, S(O)ₘ C₃₋₈ cycloalkyl, S(O)ₘ aryl, S(O)ₘ heteroaryl, S(O)ₘN(R^{a})₂, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, aryl and heterocyclyl;

R⁴, R⁵, R⁶, and R⁷ are independently selected from absent, hydrogen, CF₃, CN, halo, CO₂H, OH, C₁₋₆ alkyl, S(O)ₘ C₁₋₆ alkyl, N(R^{a})₂, C(═O)N(R^{a})₂, S(O)ₘN(R^{a})₂, C₃₋₈ cycloalkyl, C₃₋₈ cycloalkenyl, aryl, heteroaryl and heterocyclyl, wherein said alkyl, cycloalkyl, cycloalkenyl, aryl, heteroaryl and heterocyclyl are optionally substituted with one to four substituents selected from CF₃, halo, N(R^{a})₂, C(═O)N(R^{a})₂, OH, (O)C₁₋₆ alkyl, C₁₋₆ alkyl, C₃₋₆ cycloalkyl, aryl, heteroaryl, and heterocyclyl, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl and C₃₋₆ cycloalkyl, and R⁴ and R⁵ or R⁵ and R⁶ can be taken together to form a C₃₋₈ cycloalkyl, C₃₋₈ cycloalkenyl, aryl, heteroaryl or heterocyclyl, wherein said alkyl, cycloalkyl, cycloalkenyl, aryl, heteroaryl and heterocyclyl are optionally substituted with one to four substituents selected from CF₃, halo, N(R^{a})₂, C(═O)N(R^{a})₂, OH, (O)C₁₋₆ alkyl, C₁₋₆ alkyl, C₃₋₆ cycloalkyl, aryl, heteroaryl, and heterocyclyl, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl and C₃₋₆ cycloalkyl;

R⁸ is hydrogen or halo;

R^{a} is independently selected from hydrogen, C₁₋₃ alkyl and C₃₋₆ cycloalkyl, wherein said alkyl and cycloalkyl are optionally substituted with CF₃, halo, N(R^{b})₂, OH, (O)C₁₋₃ alkyl, C₁₋₃ alkyl, C₃₋₆ cycloalkyl and phenyl; and

R^{b} is independently selected from hydrogen and C₁₋₃ alkyl;

or a pharmaceutically acceptable salt, tautomer or a stereoisomer thereof.

In a second embodiment of Formula B, the inhibitors of PKD1 activity are illustrated, wherein,

R³ is pyrazolyl and C₂₋₆ alkenyl which are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl;

R⁴, R⁵, R⁶ and R⁷ are independently selected from absent, hydrogen, CF₃, halo, N(R^{a})₂, C(═O)N(R^{a})₂, OH, (O)C₁₋₃ alkyl, C₁₋₃ alkyl, C₃₋₆ cycloalkyl, phenyl, and pyrrolinyl, wherein said alkyl, cycloalkyl, phenyl, and pyrrolinyl, are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl, morpholinyl, piperazinyl, piperidinyl and pyrrolidinyl said cycloalkyl, phenyl, morpholinyl, piperazinyl and pyrrolidinyl are optionally substituted with one to two substituents selected from OH and C₁₋₃ alkyl, and R⁴ and R⁵ or R⁵ and R⁶ can be taken together to form, with the phenyl to which they are attached, isoindolinyl, isoquinolinyl and indenyl, which are optionally substituted with one to two substituents selected from CF₃, halo, N(R^{a})₂, C(═O)N(R^{a})_{z}, OH, (O)C₁₋₃ alkyl, C₁₋₃ alkyl, C₃₋₆ cycloalkyl, phenyl, morpholinyl, piperazinyl, piperidinyl and pyrrolidinyl;

all other substituents are as defined in the first embodiment of Formula B;

or a pharmaceutically acceptable salt, tautomer or a stereoisomer thereof.

In a third embodiment of Formula B, the inhibitors of PKD1 activity are illustrated, wherein,

R³ is pyrazolyl which is optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl;

R⁴, R⁵, R⁶ and R⁷ are independently selected from absent, hydrogen, CF₃, halo, N(R^{a})₂, C(═O)N(R^{a})₂, OH, (O)C₁₋₃ alkyl, C₁₋₃ alkyl, C₃₋₆ cycloalkyl, phenyl, and pyrrolinyl, wherein said alkyl, cycloalkyl, phenyl, and pyrrolinyl, are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl, morpholinyl, piperazinyl, piperidinyl and pyrrolidinyl said cycloalkyl, phenyl, morpholinyl, piperazinyl and pyrrolidinyl are optionally substituted with one to two substituents selected from OH and C₁₋₃ alkyl, and R⁴ and R⁵ or R⁵ and R⁶ can be taken together to form, with the phenyl to which they are attached, isoindolinyl, isoquinolinyl and indenyl, which are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl, morpholinyl, piperazinyl, piperidinyl and pyrrolidinyl;

all other substituents are as defined in the second embodiment of Formula B;

or a pharmaceutically acceptable salt, tautomer or a stereoisomer thereof.

In a fourth embodiment of Formula B, the inhibitors of PKD1 activity are illustrated, wherein,

R³ is pyrazolyl which is optionally substituted with CF₃, C₁₋₃ alkyl, phenyl and heteroaryl, wherein said alkyl is optionally substituted with phenyl or heterocyclyl;

R⁴, R⁵, R⁶ and R⁷ are independently selected from absent, hydrogen, CF₃, halo, N(R^{a})₂, C(═O)N(R^{a})₂, OH, (O)C₁₋₃ alkyl, C₁₋₃ alkyl, C₃₋₆ cycloalkyl, phenyl, and pyrrolinyl, wherein said alkyl, cycloalkyl, phenyl, and pyrrolinyl, are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl, morpholinyl, piperazinyl, piperidinyl and pyrrolidinyl said cycloalkyl, phenyl, morpholinyl, piperazinyl and pyrrolidinyl are optionally substituted with one to two substituents selected from OH and C₁₋₃ alkyl, and R⁴ and R⁵ or R⁵ and R⁶ can be taken together to form, with the phenyl to which they are attached, isoindolinyl, isoquinolinyl and indenyl, which are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl, morpholinyl, piperazinyl, piperidinyl and pyrrolidinyl;

all other substituents are as defined in the third embodiment of Formula B;

or a pharmaceutically acceptable salt, tautomer or a stereoisomer thereof.

In a third embodiment of this invention, the inhibitors of PDK1 activity are illustrated by the Formula C:

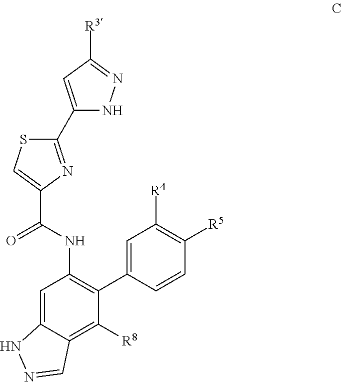

wherein:

R^{3′} is hydrogen or CF₃;

R⁴ and R⁵ are independently selected from hydrogen and C₁₋₃ alkyl, said alkyl is substituted with NH₂, or R⁴ and R⁵ can be taken together to form, with the phenyl to which they are attached, isoindolinyl; and

R⁸ is hydrogen or fluoro;

or a pharmaceutically acceptable salt, tautomer or a stereoisomer thereof.

Specific compounds of the instant invention include:
N-(5-(isoindolin-5-yl)-1H-indazol-6-yl)-2-(3-(trifluoromethyl)-1H-pyrazol-5-yl)thiazole-4-carboxamide (1-7);
N-(5-(4-(aminomethyl)phenyl)-1H-indazol-6-yl)-2-(3-(trifluoromethyl)-1H-pyrazol-5-yl)thiazole-4-carboxamide (1-8);
N-(5-(3-(aminomethyl)phenyl)-1H-indazol-6-yl)-2-(3-(trifluoromethyl)-1H-pyrazol-5-yl)thiazole-4-carboxamide (1-9);
(R)—N-(5-(3-(1-aminoethyl)phenyl)-1H-indazol-6-yl)-2-(3-(trifluoromethyl)-1H-pyrazol-5-yl)thiazole-4-carboxamide (1-10);
N-(5-(3-(pyrrolidin-1-ylmethyl)phenyl)-1H-indazol-6-yl)-2-(3-(trifluoromethyl)-1H-pyrazol-5-yl)thiazole-4-carboxamide (1-11);
N-(5-(3-(aminomethyl)-4-methylphenyl)-1H-indazol-6-yl)-2-(3-(trifluoromethyl)-1H-pyrazol-5-yl)thiazole-4-carboxamide (1-12);
(S)—N-(5-(3-(1-aminoethyl)phenyl)-1H-indazol-6-yl)-2-(3-(trifluoromethyl)-1H-pyrazol-5-yl)thiazole-4-carboxamide (1-13);
N-(5-(5-(aminomethyl)-2-fluorophenyl)-1H-indazol-6-yl)-2-(3-(trifluoromethyl)-1H-pyrazol-5-yl)thiazole-4-carboxamide (1-14);
N-(5-(3-((methylamino)methyl)phenyl)-1H-indazol-6-yl)-2-(3-(trifluoromethyl)-1H-pyrazol-5-yl)thiazole-4-carboxamide (1-15);
N-(5-(3-(2-aminopropan-2-yl)phenyl)-1H-indazol-6-yl)-2-(3-(trifluoromethyl)-1H-pyrazol-5-yl)thiazole-4-carboxamide (1-16);
N-(5-(3-(morpholinomethyl)phenyl)-1H-indazol-6-yl)-2-(3-(trifluoromethyl)-1H-pyrazol-5-yl)thiazole-4-carboxamide (1-17);
N-(5-(4-(1-aminocyclopropyl)phenyl)-1H-indazol-6-yl)-2-(3-(trifluoromethyl)-1H-pyrazol-5-yl)thiazole-4-carboxamide (1-18);
N-(5-(3-(aminomethyl)-2-fluorophenyl)-1H-indazol-6-yl)-2-(3-(trifluoromethyl)-1H-pyrazol-5-yl)thiazole-4-carboxamide (1-19);
N-(5-(3-((dimethylamino)methyl)phenyl)-1H-indazol-6-yl)-2-(3-(trifluoromethyl)-1H-pyrazol-5-yl)thiazole-4-carboxamide (1-20);
N-(5-(2-methylisoindolin-5-yl)-1H-indazol-6-yl)-2-(3-(trifluoromethyl)-1H-pyrazol-5-yl)thiazole-4-carboxamide (1-21);
N-(5-(3-((4-methylpiperazin-1-yl)methyl)phenyl)-1H-indazol-6-yl)-2-(3-(trifluoromethyl)-1H-pyrazol-5-yl)thiazole-4-carboxamide (1-22);
N-(5-(3-(1-aminocyclopropyl)phenyl)-1H-indazol-6-yl)-2-(3-(trifluoromethyl)-1H-pyrazol-5-yl)thiazole-4-carboxamide (1-23);
N-(5-(3-oxoisoindolin-5-yl)-1H-indazol-6-yl)-2-(3-(trifluoromethyl)-1H-pyrazol-5-yl)thiazole-4-carboxamide (1-24);
N-(5-(1,2,3,4-tetrahydroisoquinolin-7-yl)-1H-indazol-6-yl)-2-(3-(trifluoromethyl)-1H-pyrazol-5-yl)thiazole-4-carboxamide (1-25);
N-(5-(3-(2-aminoethyl)phenyl)-1H-indazol-6-yl)-2-(3-(trifluoromethyl)-1H-pyrazol-5-yl)thiazole-4-carboxamide (1-26);
N-(5-(2-amino-2,3-dihydro-1H-inden-5-yl)-1H-indazol-6-yl)-2-(3-(trifluoromethyl)-1H-pyrazol-5-yl)thiazole-4-carboxamide (1-27);
N-(5-(3-(pyrrolidin-2-yl)phenyl)-1H-indazol-6-yl)-2-(3-(trifluoromethyl)-1H-pyrazol-5-yl)thiazole-4-carboxamide (1-28);
N-(4-fluoro-5-(isoindolin-5-yl)-1H-indazol-6-yl)-2-(3-(trifluoromethyl)-1H-pyrazol-5-yl)thiazole-4-carboxamide (1-29);
N-(5-(isoindolin-5-yl)-1H-indazol-6-yl)-2-(1H-pyrazol-4-yl)thiazole-4-carboxamide (2-3);
N-(5-(4-(aminomethyl)phenyl)-1H-indazol-6-yl)-2-(1H-pyrazol-4-yl)thiazole-4-carboxamide (2-5);
N-(5-(3-(aminomethyl)phenyl)-1H-indazol-6-yl)-2-(1H-pyrazol-4-yl)thiazole-4-carboxamide (2-6);
N-(5-(3-(piperidin-1-ylmethyl)phenyl)-1H-indazol-6-yl)-2-(3-(trifluoromethyl)-1H-pyrazol-5-yl)thiazole-4-carboxamide (3-5);
N-(5-(3-((2-(dimethylamino)ethylamino)methyl)phenyl)-1H-indazol-6-yl)-2-(3-(trifluoromethyl)-1H-pyrazol-5-yl)thiazole-4-carboxamide (3-6);
N-(5-(3-((benzylamino)methyl)phenyl)-1H-indazol-6-yl)-2-(3-(trifluoromethyl)-1H-pyrazol-5-yl)thiazole-4-carboxamide (3-7);
N-(5-(3-((cyclopropylamino)methyl)phenyl)-1H-indazol-6-yl)-2-(3-(trifluoromethyl)-1H-pyrazol-5-yl)thiazole-4-carboxamide (3-8);
N-(5-(3-((2-methoxyethylamino)methyl)phenyl)-1H-indazol-6-yl)-2-(3-(trifluoromethyl)-1H-pyrazol-5-yl)thiazole-4-carboxamide (3-9);
N-(5-(3-((3-aminopyrrolidin-1-yl)methyl)phenyl)-1H-indazol-6-yl)-2-(3-(trifluoromethyl)-1H-pyrazol-5-yl)thiazole-4-carboxamide (3-10);
N-(5-(3-((3-hydroxypyrrolidin-1-yl)methyl)phenyl)-1H-indazol-6-yl)-2-(3-(trifluoromethyl)-1H-pyrazol-5-yl)thiazole-4-carboxamide (3-11);
N-(5-(3-((2-aminoethylamino)methyl)phenyl)-1H-indazol-6-yl)-2-(3-(trifluoromethyl)-1H-pyrazol-5-yl)thiazole-4-carboxamide (3-12);
N-(5-(3-((2-(methylamino)ethylamino)methyl)phenyl)-1H-indazol-6-yl)-2-(3-(trifluoromethyl)-1H-pyrazol-5-yl)thiazole-4-carboxamide (3-13);
N-(5-(3-((2-hydroxyethylamino)methyl)phenyl)-1H-indazol-6-yl)-2-(3-(trifluoromethyl)-1H-pyrazol-5-yl)thiazole-4-carboxamide (3-14);
N-(5-(6-(aminomethyl)pyridin-3-yl)-1H-indazol-6-yl)-2-(3-(trifluoromethyl)-1H-pyrazol-5-yl)thiazole-4-carboxamide (4-4);
N-(5-(4-(aminomethyl)-2-methylphenyl)-1H-indazol-6-yl)-2-(1H-pyrazol-4-yl)thiazole-4-carboxamide (4-5);
N-(5-(4-(aminomethyl)-2-methylphenyl)-1H-indazol-6-yl)-2-(3-(trifluoromethyl)-1H-pyrazol-5-yl)thiazole-4-carboxamide (4-6);
N-(5-(3-(aminomethyl)-4-fluorophenyl)-1H-indazol-6-yl)-2-(3-(trifluoromethyl)-1H-pyrazol-5-yl)thiazole-4-carboxamide (4-7);
N-(5-(3-(aminomethyl)phenyl)-1H-indazol-6-yl)-2-(3-phenyl-1H-pyrazol-5-yl)thiazole-4-carboxamide (5-6);
N-(5-(isoindolin-5-yl)-1H-indazol-6-yl)-2-(3-methyl-1H-pyrazol-5-yl)thiazole-4-carboxamide (5-7);
N-(5-(3-(aminomethyl)phenyl)-1H-indazol-6-yl)-2-(1-benzyl-1H-pyrazol-4-yl)thiazole-4-carboxamide hydrochloride (8-5);
N-(2-(2-(aminomethyl)thiazol-4-yl)-4-fluorophenyl)-2-(1H-pyrazol-4-yl)thiazole-4-carboxamide hydrochloride (10-6);
2-(cyclopropylethynyl)-N-(5-(isoindolin-5-yl)-1H-indazol-6-yl)thiazole-4-carboxamide hydrochloride (13-5);
N-(5-(3-(aminomethyl)-2-fluorophenyl)-1H-indazol-6-yl)-2-(3-methyl-1H-pyrazol-5-yl)thiazole-4-carboxamide hydrochloride (15-3);
N-(4′-(aminomethyl)-5-fluorobiphenyl-2-yl)-2-(1H-pyrazol-4-yl)thiazole-4-carboxamide (18-4); and
N-(2-(4-(aminomethyl)cyclohex-1-enyl)-4-fluorophenyl)-2-(1H-pyrazol-4-yl)thiazole-4-carboxamide (19-9);
   or a pharmaceutically acceptable salt, tautomer or a stereoisomer thereof.

All compounds were made and isolated as the hydrochloride salt.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention.

In addition, the compounds disclosed herein may exist as tautomers and both tautomeric forms are intended to be encompassed by the scope of the invention, even though only one tautomeric structure is depicted.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off target activity, packaging properties, and so on. (Diass, J. O. et al. Organometallics (2006) 5:1188-1198; Showell, G. A. et al. Bioorganic & Medicinal Chemistry Letters (2006) 16:2555-2558).

In the compounds of generic Formula A, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in viva half-life or reducing dosage requirements, or may provide a compound useful as a standard for characterization of biological samples. Isotopically-enriched compounds within generic Formula A can be prepared without undue experimentation by conventional techniques well known to those skilled in the art or by processes analogous to those described in the Schemes and Examples herein using appropriate isotopically-enriched reagents and/or intermediates.

As used herein, “alkyl” is intended to include both branched and straight-chain saturated aliphatic hydrocarbon groups having the specified number of carbon atoms. For example, C₁-C₆, as in “C₁-C₆ alkyl” is defined to include groups having 1, 2, 3, 4, 5 or 6 carbons in a linear or branched arrangement. For example, “C₁-C₆ alkyl” specifically includes methyl, ethyl, n-propyl, i-propyl, n-butyl, t-butyl, i-butyl, pentyl, hexyl, and so on.

The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on.

If no number of carbon atoms is specified, the term “alkenyl” refers to a non-aromatic hydrocarbon radical, straight, branched or cyclic, containing from 2 to 6 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 6 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydro-naphthyl, indanyl and biphenyl. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrazolyl, indolyl, benzotriazolyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively. Such heteroaryl moieties for substituent Q include but are not limited to: 2-benzimidazolyl, 2-quinolinyl, 3-quinolinyl, 4-quinolinyl, 1-isoquinolinyl, 3-isoquinolinyl and 4-isoquinolinyl.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzoimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As appreciated by those of skill in the art, “halo” or “halogen” as used herein is intended to include chloro (Cl), fluoro (F), bromo (Br) and iodo (I).

In an embodiment of Formula A, p is 0, 1 or 2.

In an embodiment of Formula A, p is 0.

In an embodiment of Formula A, ring Z is attached to phenyl via a carbon-carbon bond and is selected from cyclohexenyl, pyridyl, isoindolinyl, isoquinolinyl, indenyl and phenyl.

In an embodiment of Formula A, ring Z is attached to phenyl via a carbon-carbon bond and is phenyl.

In another embodiment of Formula A, R¹ and R² are independently selected from halo, OH, C₁₋₃ alkyl, cyclopropyl and NH₂, or R¹ and R² can be taken together to form an imidazole, pyrrole, pyrazole, thiophene or furan.

In another embodiment of Formula A, R¹ and R² are taken together to form an imidazole, pyrrole, pyrazole, thiophene or furan.

In another embodiment of Formula A, R³ is pyrazolyl which is optionally substituted with one to two substituents selected from CF₃, halo, N(R^{a})₂, (C═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl.

In another embodiment of Formula A, R³ is pyrazolyl, said pyrazolyl is optionally substituted with one to two substituents selected from CF₃, halo, N(R^{a})₂, C(═O)N(R^{a})₂, OH, (O)C₁₋₃ alkyl, C₁₋₃ alkyl and C₃₋₆ cycloalkyl.

In another embodiment of Formula A, R⁴ is independently selected from CF₃, CN, halo, CO₂H, OH, S(O)ₘ C₁₋₆ alkyl, S(O)ₘ N(R^{a})₂, C(═O)N(R^{a})₂, N(R^{a})₂, C₁₋₆ alkyl, C₃₋₈ cycloalkyl, C₃₋₈ cycloalkenyl, aryl, heteroaryl and heterocyclyl, wherein said alkyl, cycloalkyl, cycloalkenyl, aryl, heteroaryl and heterocyclyl are optionally substituted with one to four substituents selected from CF₃, halo, N(R^{a})₂, C(═O)N(R^{a})₂, OH, (O)C₁₋₃ alkyl, C₁₋₃ alkyl, C₃₋₆ cycloalkyl and C(O)ₘN(R^{b})₂.

In another embodiment of Formula A, R⁴ is independently selected from hydrogen, halo, OH, C₁₋₃ alkyl and heterocyclyl, said alkyl and heterocyclyl are optionally substituted with one to two substituents selected from N(R^{a})₂, C₃₋₆ cycloalkyl and heterocyclyl, said alkyl, cycloalkyl and heterocyclyl are optionally substituted with one to two substituents selected from N(R^{b})₂, and OH.

In another embodiment of Formula A, R⁴ is independently selected from hydrogen, halo, OH, C₁₋₃ alkyl and pyrrolidinyl, said alkyl and pyrrolidinyl are optionally substituted with one to two substituents selected from N(R^{a})₂, pyrrolidinyl, morpholinyl, cyclopropyl and piperidinyl, said pyrrolidinyl, morpholinyl, cyclopropyl and piperidinyl are optionally substituted with one to two substituents selected from N(R^{b})₂ and OH.

In another embodiment of Formula A, R⁴ is independently selected from hydrogen, halo and C₁₋₃ alkyl, said alkyl is substituted with NH₂.

In another embodiment of Formula A, R⁴ is independently selected from hydrogen and C₁₋₃ alkyl, said alkyl is substituted with NH₂.

In another embodiment of Formula A, R⁸ is hydrogen.

In another embodiment of Formula A,

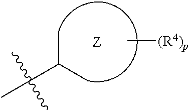

is selected from

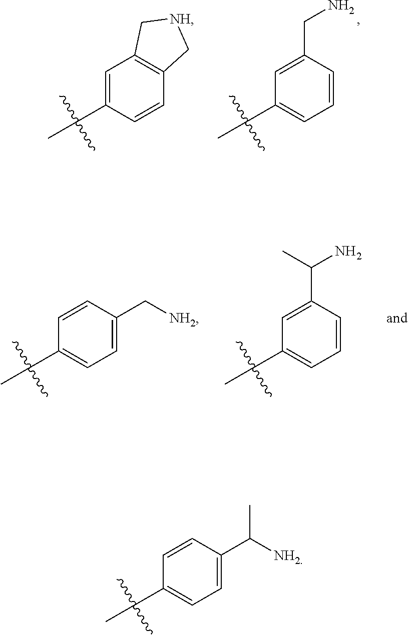

In another embodiment of Formula A,

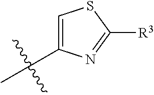

is

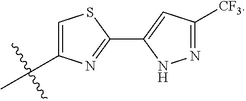 [SMILES: *c1csc(-c2cc(C(F)(F)F)n[nH]2)n1]

Included in the instant invention is the free form of compounds of Formula A, as well as the pharmaceutically acceptable salts and stereoisomers thereof. Some of the isolated specific compounds exemplified herein are the protonated salts of amine compounds. The term “free form” refers to the amine compounds in non-salt form. The encompassed pharmaceutically acceptable salts not only include the isolated salts exemplified for the specific compounds described herein, but also all the typical pharmaceutically acceptable salts of the free form of compounds of Formula A. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic (TFA) and the like.

When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

### Utility

3-Phosphoinositide-dependent protein kinase-1 (PDK1) is a 556-amino acid containing enzyme comprised of a C-terminal Pleckstrin homology (PH) domain (residues 459-550) and an N-terminal kinase domain (residues 70-359). The PH domain of PDK1 binds phosphatidylinositols (e.g., phosphatidylinositol 4,5-bisphosphate and phosphatidylinositol 3,4,5-triphosphate) produced by phosphatidylinositol kinases, such as phosphatidylinositol 3-kinase (PI3K) and whose levels are, in part, controlled by phosphatases such as PTEN (phosphatase and tensin homologue). PDK1 plays a central role in the PI3K/Akt pathway and has been called a “master regulator” kinase due to its role as a critical upstream activating kinase that phosphorylates the so-called T-loop phosphorylation site for multiple kinases in the AGC family of kinases including but not limited to all three isoforms of PKB (PKBα, PKBβ, PKBγ, also known as Akt1, Akt2, and Akt3, respectively), RSK (three isoforms RSK1, RSK2, RSK3, also known as p90RSK), p70S6K (two isoforms, S6K1 and S6K2), SGK1 and PKC.

Signals from several peptide growth factors including insulin, insulin-like growth factor-1 and platelet-derived growth factor are transduced by PKB. Like PDK1, PKB contains a PH domain that binds phosphatidyl 3,4,5-triphosphate. PKB is translocated to the plasma membrane and phosphorylated by PDK1 at residue T-308/309 (the two phosphosites correspond to different isoforms) in response to the second messenger phosphatidyl 3,4,5-triphosphate produced by PI3K. Activation of PKB in tumor cells results in increased cellular survival via anti-apoptotic signals and also proliferation. PKB amplification has been observed in a proportion of several tumor types including ovarian, breast and pancreatic cancers. Similarly, PKBα amplification has been observed in a percentage of gastric adenocarcinoma samples. Recently, an activated mutant form of PKBα (E 17K) was detected in a number of breast (8%), colorectal (6%), and ovarian (2%) cancers. PDK1 kinase inhibitors are useful as treatments for diseases linked to PKB signaling (such as cancer, Cowden syndrome, Lhermitte-Dudos disease and Bannayan-Zonana syndrome) by preventing activation of PKB signaling by PDK1.

Similarly, PDK1 kinase inhibitors are useful for treating cancer or other proliferative disorders by blocking the activation of p70S6K by PDK1. There are several substrates of p70S6K, such as ribosomal S6 protein, eIF4B, PDCD4 etc., that are involved in translation inhibition complex formation or ribosomal protein synthesis. Inhibition of protein synthesis via inhibition of phosphorylation of ribosomal S6 protein is believed to inhibit the proliferation of tumor cells by mTOR inhibitors (e.g., rapamycin). p70S6K gene amplification has been observed in breast tumor specimens, Simultaneous amplification of p70S6K and HER-2 correlates with poor survival in cancer patients. Hyperactivation of p70S6K (as measured by phosphorylation of T389) has been observed by immunohistochemical analysis of breast, head and neck squamous cell carcinoma (HNSCC), glioblastoma, lung and liver primary tumor specimens.

Likewise, PDK1 kinase inhibitors are useful for the treatment of cancer by blocking the activation of RSK1 (also known as p90RSK) by PDK1. RSK1 transduces anti-apoptotic and proliferative signals be mediating phosphorylation directly or indirectly of BAD, LKB1, TSC2, NFkB, mTOR. Ras/MAPK pathway is activated in >50% in primary tumors. RSK1 activity is correlated with MAPK activity. RSK1 is overexpressed in primary breast and prostate cancer samples.

PDK1 signaling regulates multiple critical steps in angiogenesis. Inhibitors of the activity of PDK1 are thus useful in the treatment of cancer, in particular cancers associated with deregulated activity of the PTEN/PI3K pathway including, but not limited to PTEN loss of function mutations, PI3K gain of function mutations and receptor tyrosine kinase gain of function mutations.

PDK1 signaling has also been implicated in tumorigenesis and a PDK1 inhibitor is useful for tumor prevention or prevention of tumor recurrence. Mice with a PTEN heterozygous (PTEN^{+/-}) genotype are well-known to spontaneously develop tumors. Alessi and co-workers found that PDK1 hypomorphic PTEN^{+/-} mice expressing <30% of normal PDK1 protein levels showed a significant delay in tumor formation as compared to littermate controls expressing normal levels of PDK1 protein (Current Biology, 2005, 15, 1839-1846).

SGK1 (serum and glucocorticoid-regulated kinase-1) activity is critical for insulin-mediated Na+ retention and hypertensive effects. Inhibition of SGK1 activity by a PDK1 kinase inhibitor is useful in treating hypertension and/or hypoinsulinemia.

The compounds of the instant invention are useful for treating tumor recurrence.

The compounds of the instant invention are useful for treating hypertension.

The compounds of the instant invention are useful for treating diabetes mellitus.

The instant invention provides a method for treating tumor recurrence that comprises administering such inhibitors of PDK1 activity.

The instant invention provides a method for treating hypertension that comprises administering such inhibitors of PDK1 activity.

The instant invention provides a method for treating diabetes mellitus that comprises administering such inhibitors of PDK1 activity.

The compounds, compositions and methods provided herein are particularly deemed useful for the treatment of cancer. Cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

Cancers that may be treated by the compounds, compositions and methods of the invention include, but are not limited to: breast, prostate, colon, colorectal, lung, brain, testicular, stomach, pancrease, skin, small intestine, large intestine, throat, head and neck, oral, bone, liver, bladder, kidney, thyroid and blood.

Cancers that may be treated by the compounds, compositions and methods of the invention include breast, prostate, colon, ovary, endometrium and thyroid.

Cancers that may be treated by the compounds, compositions and methods of the invention include breast and prostate.

The compounds of the invention are also useful in preparing a medicament that is useful in treating cancer.

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bendamustine hydrochloride (Treanda®); bevacuzimab (Avastin®); bexarotene capsules (Targretin®); bexarotene gel (Targretin®); bleomycin (Blenoxane®); bortezomib (Velcade®); brefeldin A; busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); dalteparin sodium injection (Fragmin®); Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); degarelix (Firmagon®); Denileukin diftitox (Ontak®); dexrazoxane (Zinecard®); dexrazoxane hydrochloride (Totect®); didemnin B; 17-DMAG; docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); eculizumab injection (Soliris®); Elliott's B Solution (Elliott's B Solution®); eltrombopag (Promacta®); epirubicin (Ellence®); Epoetin alfa (Epogen®); erlotinib (Tarceva®); estramustine (Emcyt®); ethinyl estradiol; etoposide phosphate (Etopophos®); etoposide, VP-16 (Vepesid®); everolimus tablets (Afinitor®); exemestane (Aromasin®); ferumoxytol (Feraheme Injection®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); geldanamycin; gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); iobenguane I 123 injection (AdreView®); irinotecan (Camptosar®); ixabepilone (Ixempra®); lapatinib tablets (Tykerb®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); 8-methoxypsoralen; mitomycin C (Mutamycin®); mitotane (Lysodren®); mitoxantrone (Novantrone®); mitramycin; nandrolone phenpropionate (Durabolin-50®); nelarabine (Arranon®); nilotinib (Tasigna®); Nofetumomab (Verluma®); ofatumumab (Arzerra®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pazopanib tablets (Votrienttm®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plerixafor (Mozobil®); plicamycin, mithramycin (Mithracin®); porfimer sodium (Photofrin®); pralatrexate injection (Folotyn®); procarbazine (Matulane®); quinacrine (Atabrine®); rapamycin; Rasburicase (Elitek®); raloxifene hydrochloride (Evista®); Rituximab (Rituxan®); romidepsin (Istodax®); romiplostim (Nplate®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); testolactone (Teslac®); thioguanine, 6-TG (Thioguanine®); thiopurine; thiotepa (Thioplex®); topotecan (Hycamtin®); toremifene (Fareston®); Tositumomab (Bexxar®); Tositumomab/I-131 tositumomab (Bexxar®); trans-retinoic acid; Trastuzumab (Herceptin®); tretinoin, ATRA (Vesanoid®); triethylenemelamine; Uracil Mustard (Uracil Mustard Capsules®); valrubicin (Valstar®); vinblastine (Velban®); vincristine (Oncovin®); vinorelbine (Navelbine®); vorinostat (Zolinza®); wortmannin; and zoledronate (Zometa®).

The compounds of the instant invention are useful for treating cancer in combination with taxanes.

The compounds of the instant invention are useful for treating cancer in combination with docetaxel (Taxotere®).

The compounds of the instant invention are useful for treating cancer in combination with vorinostat (Zolinza®).

The compounds of the instant invention are useful for treating cancer in combination with the mTor inhibitor, AP 23573.

The compounds of the instant invention are useful for treating cancer in combination with the Akt inhibitor, MK-2206.

The compounds of the instant invention are useful for treating cancer in combination with the Akt inhibitor, MK-8152.

The compounds of the instant invention are useful for treating cancer in combination with the IGF1R inhibitor, MK-0646.

The compounds of the instant invention are useful for treating cancer in combination with satraplatin.

The compounds of the instant invention are useful for treating cancer in combination with lapatinib (Tykerb®).

The compounds of this invention may be administered to mammals, including humans, either alone or, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula A may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula A are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a composition according to this invention is administered into a human subject, the daily dosage will normally be determined by the prescribing physician with the dosage generally varying according to the age, weight, and response of the individual patient, as well as the severity of the patient's symptoms.

In an embodiment, a suitable amount of an inhibitor of PDK1 is administered to a mammal undergoing treatment for cancer. Administration occurs in an amount of inhibitor of between about 0.1 mg/kg of body weight to about 60 mg/kg of body weight per day, or between 0.5 mg/kg of body weight to about 40 mg/kg of body weight per day. Another therapeutic dosage that comprises the instant composition includes from about 0.01 mg to about 1000 mg of inhibitor of PDK1. In another embodiment, the dosage comprises from about 1 mg to about 1000 mg of inhibitor of PDK1.

The instant compounds are also useful in combination with therapeutic, chemotherapeutic and anti-cancer agents. Combinations of the presently disclosed compounds with therapeutic, chemotherapeutic and anti-cancer agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints. The instant compounds are particularly useful when co-administered with radiation therapy.

Thus, the scope of the instant invention encompasses the use of the instantly claimed compounds in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The instant invention also includes a pharmaceutical composition useful for treating or preventing cancer that comprises a therapeutically effective amount of a compound of the instant invention and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

All patents, publications and pending patent applications identified are hereby incorporated by reference.

Abbreviations used in the description of the chemistry and in the Examples that follow are: DMF: N,N-dimethylformamide; DIPEA: N,N-diisopropylethylamine; TFA: trifluoroacetic acid; DMSO: dimethylsulfoxide; TLC: thin layer chromatography; HPLC: high pressure liquid chromatography; THF: tetrahydrofuran; HATU: O-(7-azabenzotriazol-1-yl)-N,N,N′,N′-tetramethyluronium hexafluorophosphate; SEMC1: 2-(trimethylsilyl)ethoxymethyl chloride.

The compounds of this invention may be prepared by employing reactions as shown in the following Reaction Schemes, in addition to other standard manipulations that are known in the literature or exemplified in the experimental procedures. The illustrative Reaction Schemes below, therefore, are not limited by the compounds listed or by any particular substituents employed for illustrative purposes. Substituent numbering as shown in the Reaction Schemes do not necessarily correlate to that used in the claims and often, for clarity, a single substituent is shown attached to the compound where multiple substituents are optionally allowed under the definitions of Formula A hereinabove.

### Synopsis of Reaction Schemes

Utilizing the following general Reaction Schemes, Reaction Schemes I-IV, one of ordinary skill in the art would be able to synthesize the substituted bicyclic molecules (see Formula A) of the instant invention. The requisite intermediates are in some cases commercially available or can be prepared according to literature procedures.
1. Zhu, G.-D.; Gong, J.; Gandhi, V. B.; Woods, K.; Luo, Y.; Liu, X.; Gaan, R.; Klinghifer, V.; Johnson, E. F.; Stoll, V. S.; Mamo, M.; Li, Q.; Rosenberg, S. H.; Giranda, V. L. Bioorg. & Med. Chem. 2007, 15, 2441-2452.
2. WO2005/070932
3. WO2005/073205
4. Bertus, P.; and Szymoniak, J. J. Org. Chem. 2003, 68, 7133-7136.

As illustrated in Reaction Scheme I, an aryl bromide such as I-1 is functionalized using methods familiar to one of ordinary skill in the art, in this case with an aryl ring using a palladium-catalyzed coupling reaction, to give I-2. Reduction of the nitro group utilizing standard reduction conditions provides I-3. Coupling of the aniline with carboxylic acids, in this case a thiazole carboxylic acid using standard techniques provides I-5.

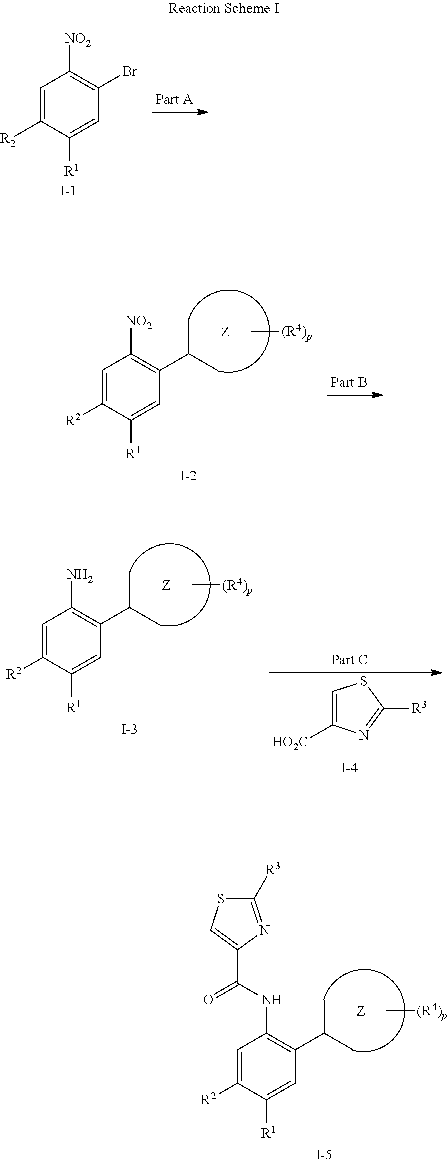

As illustrated in Reaction Scheme II, an alternative reaction sequence involves functionalization of I-1 with 3-formylphenylboronic acid using a palladium-catalyzed coupling to give II-1. Functionalization of aldehyde II-1 with an amine and a reducing agent affords II-2. Reduction of the nitro group on II-2 using standard reducing agents gives aniline II-3. Coupling of the aniline with carboxylic acids, in this case a thiazole carboxylic acid using standard techniques provides II-4.

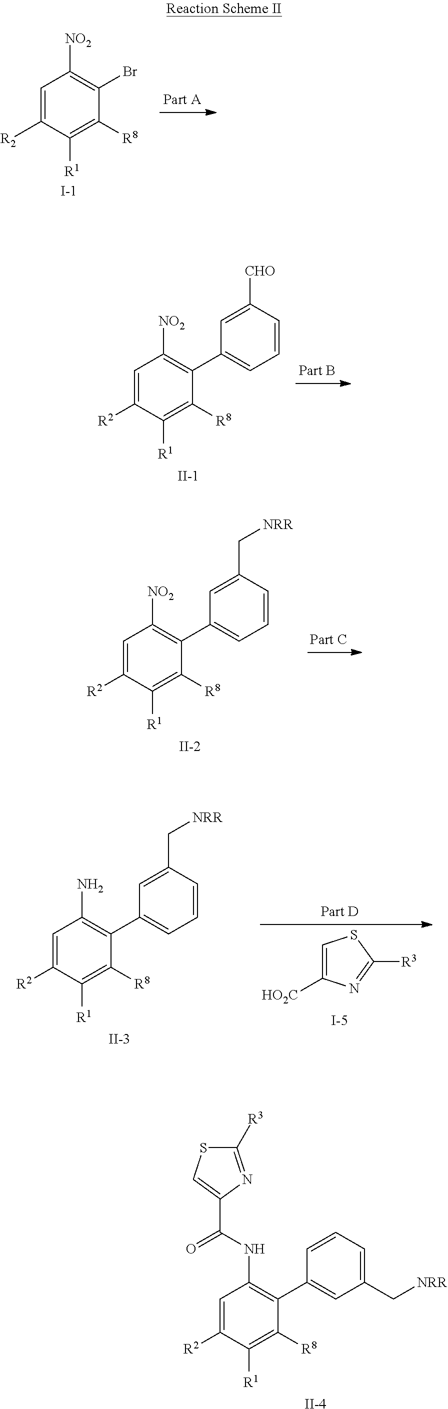

Another possible reaction sequence as shown in Reaction Scheme III involves a palladium-catalyzed coupling of I-1 with a cyano-substituted aryl boronate to give III-1. Exposure of III-1 to reducing conditions affords amine III-2. The primary amine is protected, in this case as a tert-butyl carbamate to give III-3. Subsequent coupling of III-3 with I-4 using standard conditions, followed by removal of the protecting group, in this case under acidic conditions, gave III-4.

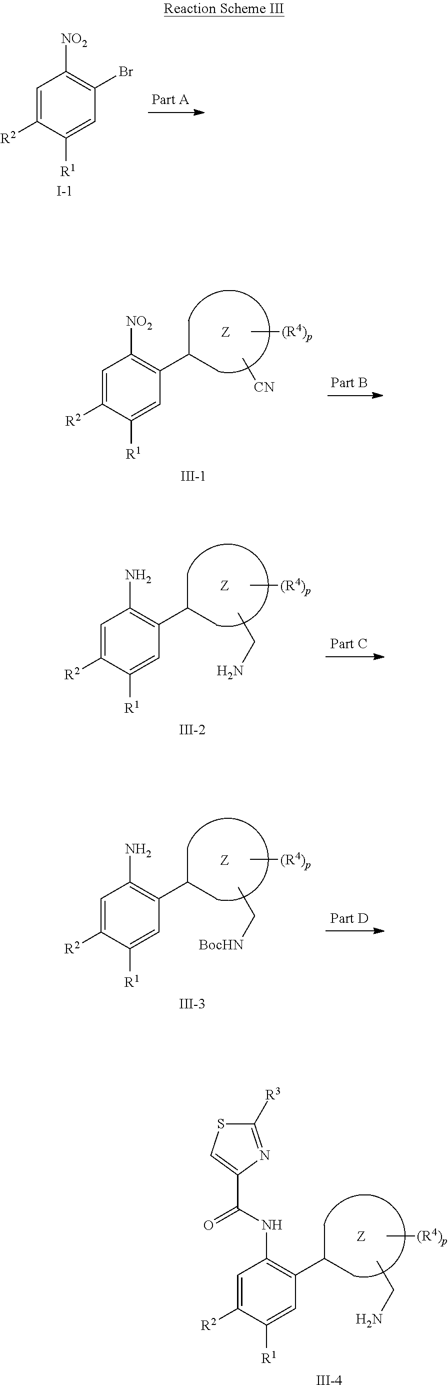

Another reaction sequence couples the aniline I-3 with carboxylic acid IV-1, using standard techniques providing IV-2. This compound is functionalized via a palladium catalyzed coupling, in this case with stannane IV-3a or borate IV-3b, followed by deprotection, in this case under acidic conditions to give IV-4.

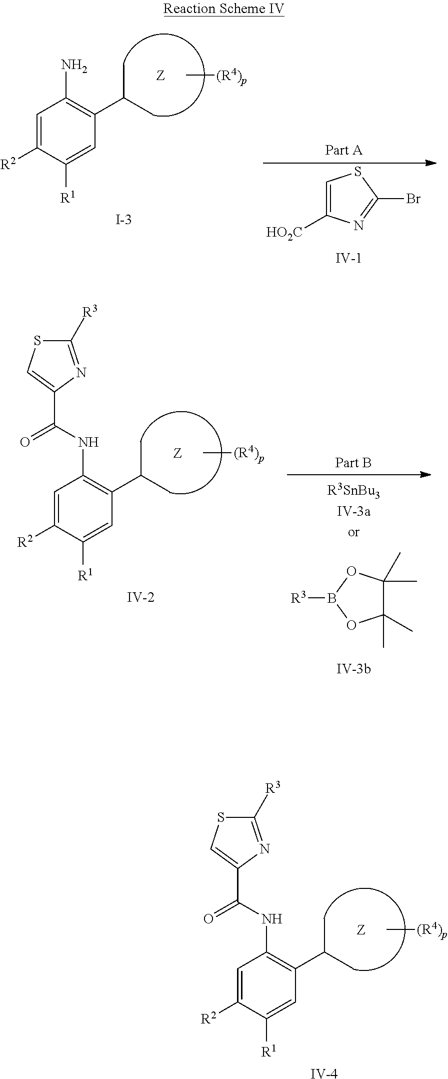

### EXAMPLES

Examples and schemes provided are intended to assist in a further understanding of the invention. Particular materials employed, species and conditions are intended to be further illustrative and do not limit the reasonable scope thereof.

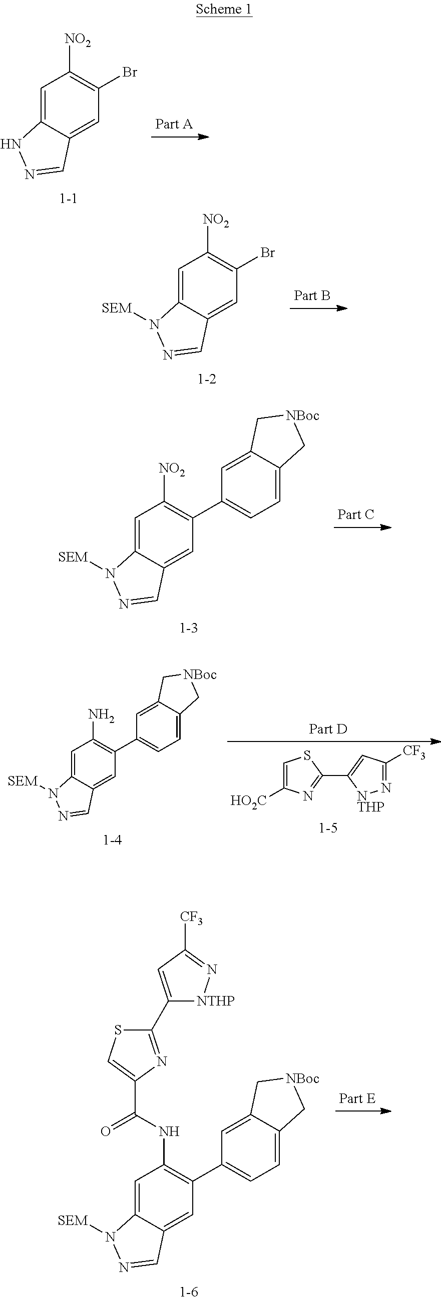

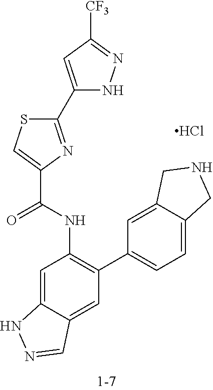 [SMILES: O=C(Nc1cc2[nH]ncc2cc1-c1ccc2c(c1)CNC2)c1csc(-c2cc(C(F)(F)F)n[nH]2)n1 ; Cl]

### N-(5-(isoindolin-5-yl)-1H-indazol-6-yl)-2-(3-(trifluoromethyl)-1H-pyrazol-5-yl)thiazole-4-carboxamide Hydrochloride (1-7)

### 5-bromo-6-nitro-1-((2-(trimethylsilyl)ethoxy)methyl)-1H-indazole (1-2)

Part A: A mixture of 5-bromo-6-nitro-1H-indazole (1-1)¹ (1.00 g, 4.13 mmol), SEMC1 (1.6 mL, 9.09 mmol) and K₂CO₃ (6.73 g, 20.7 mmol) in DMSO (10 mL) was stirred at room temperature under a nitrogen atmosphere for 18 hours. The mixture was diluted with dichloromethane, washed with water and brine, dried over sodium sulfate, filtered, concentrated under vacuum, and purified by silica gel chromatography to afford 5-bromo-6-nitro-1-((2-(trimethylsilyl)ethoxy)methyl)-1H-indazole (1-2).

### tert-butyl 5-(6-nitro-1-((2-(trimethylsilyl)ethoxy)methyl)-1H-indazol-5-yl)isoindoline-2-carboxylate (1-3)

Part B: A mixture of 5-bromo-6-nitro-1-((2-(trimethylsilyl)ethoxy)methyl)-1H-indazole (1-2) (160 mg, 0.43 mmol), tert-butyl 5-(4,4,5,5-tetramethyl-1,3,2-dioxaborolan-2-yl)isoindoline-2-carboxylate (178 mg, 0.516 mmol), PdCl₂dppf.CH₂Cl₂ (31 mg, 0.043 mmol) and K₂CO₃ (119 mg, 0.86 mmol) in DMF (1.5 mL) was stirred at 85° C. under a nitrogen atmosphere for 18 hours. The mixture was allowed to cool to room temperature, diluted with ethyl acetate, washed with water and brine, dried over sodium sulfate, filtered, concentrated under vacuum, and purified by silica gel chromatography to afford tea-butyl 5-(6-nitro-1-((2-(trimethylsilyl)ethoxy)methyl)-1H-indazol-5-yl)isoindoline-2-carboxylate (1-3).

### tert-butyl 5-(6-amino-1-((2-(trimethylsilyl)ethoxy)methyl)-1H-indazol-5-yl)isoindoline-2-carboxylate (1-4)

Part C: A mixture of tert-butyl 5-(6-nitro-1-((2-(trimethylsilyl)ethoxy)methyl)-1H-indazol-5-yl)isoindoline-2-carboxylate (1-3) (170 mg, 0.333 mmol) and Pd/C (10% Pd, 142 mg, 0.0665 mmol) in ethyl acetate (3.5 mL) was stirred under an atmosphere of hydrogen gas at room temperature for 18 hours. The mixture was then filtered through a pad of Celite and concentrated under vacuum to afford compound tert-butyl 5-(6-amino-1-((2-(trimethylsilyl)ethoxy)methyl)-1H-indazol-5-yl)isoindoline-2-carboxylate (1-4).

### tert-butyl 5-(6-(2-(1-(tetrahydro-2H-pyran-2-yl)-3-(trifluoromethyl)-1H-pyrazol-5-yl)thiazole-4-carboxamido)-1-((2-(trimethylsilyl)ethoxy)methyl)-1H-indazol-5-yl)isoindoline-2-carboxylate (1-6)

Part D: A mixture of 2-(1-(tetrahydro-2H-pyran-2-yl)-3-(trifluoromethyl)-1H-pyrazol-5-yl)thiazole-4-carboxylic acid (1-5) (87 mg, 0.251 mmol), tert-butyl 5-(6-amino-1-((2-(trimethylsilyl)ethoxy)methyl)-1H-indazol-5-yl)isoindoline-2-carboxylate (1-4) (145 mg, 0.302 mmol), HATU (143 mg, 0.377 mmol), and DIPEA (97 mg, 0.753 mmol) in DMF (1.5 mL) was stirred at room temperature for 21 hours. The mixture was diluted with ethyl acetate, washed with saturated aqueous sodium bicarbonate and brine, dried over sodium sulfate, filtered, concentrated under vacuum, and purified by silica gel chromatography to afford compound tert-butyl 5-(6-(2-(1-(tetrahydro-2H-pyran-2-yl)-3-(trifluoromethyl)-1H-pyrazol-5-yl)thiazole-4-carboxamido)-1-((2-(trimethylsilyl)ethoxy)methyl)-1H-indazol-5-yl)isoindoline-2-carboxylate (1-6).

### N-(5-(isoindolin-5-yl)-1H-indazol-6-yl)-2-(3-(trifluoromethyl)-1H-pyrazol-5-yl)thiazole-4-carboxamide Hydrochloride (1-7)

Part E: A mixture of tert-butyl 5-(6-(2-(1-(tetrahydro-2H-pyran-2-yl)-3-(trifluoromethyl)-1H-pyrazol-5-yl)thiazole-4-carboxamido)-1-((2-(trimethylsilyl)ethoxy)methyl)-1H-indazol-5-yl)isoindoline-2-carboxylate (1-6) (164 mg, 0.202 mmol) and TFA (3 mL) in dichloromethane (2 mL) and water (5 drops) was stirred at room temperature for 1 hour. The reaction was concentrated under vacuum and purified by prep-HPLC to afford N-(5-(isoindolin-5-yl)-1H-indazol-6-yl)-2-(3-(trifluoromethyl)-1H-pyrazol-5-yl)thiazole-4-carboxamide hydrochloride (1-7) as a white solid, ¹H NMR (400 MHz, DMSO d₆) δ 14.83-14.73 (m, 1H); 13.17 (bs, 1H); 9.81 (s, 1H); 9.58 (bs, 2H); 8.62-8.38 (m, 2H); 8.11 (s, 1H); 7.73 (s, 1H); 7.61-7.54 (m, 3H); 7.20-6.98 (m, 1H); 4.53 (bs, 4H). HPLC t_{R}=4.93 min (UV_{254 nm}). Mass calculated for formula C₂₃H₁₆F₃N₇OS 495.48; observed MH⁺ (ESI MS) 496.8 (m/z).

The compounds in Table 1 were prepared according to the Reaction Schemes and Scheme 1. The free base is shown.

TABLE 1

| | | | MS | | |
|---|---|---|---|---|---|
| | | | MH⁺/MH | HPLC | |
| Cmp | Structure | MW | m/z | t_{R} | Name |
| 1-8 | 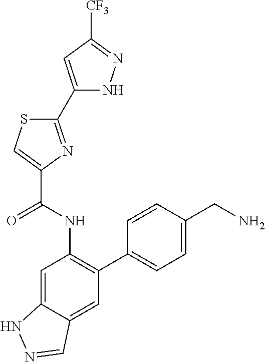 [SMILES: NCc1ccc(-c2cc3cn[nH]c3cc2NC(=O)c2csc(-c3cc(C(F)(F)F)n[nH]3)n2)cc1] | 483.1 | 482.2 | 5.48 | N-(5-(4- (aminomethyl)phenyl)- 1H-indazol-6-yl)-2- (3-(trifluoromethyl)- 1H-pyrazol-5- yl)thiazole-4- carboxamide |
| 1-9 | 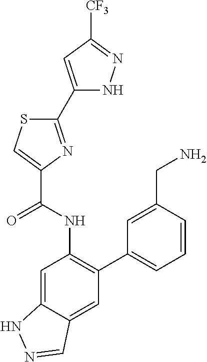 [SMILES: NCc1cccc(-c2cc3cn[nH]c3cc2NC(=O)c2csc(-c3cc(C(F)(F)F)n[nH]3)n2)c1] | 483.1 | 484.5 | 5.00 | N-(5-(3- (aminomethyl)phenyl)- 1H-indazol-6-yl)-2- (3-(trifluoromethyl)- 1H-pyrazol-5- yl)thiazole-4- carboxamide |
| 1-10 | 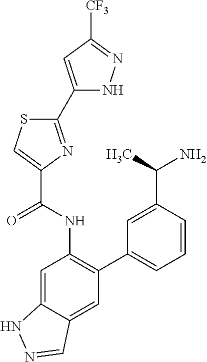 [SMILES: C[C@@H](N)c1cccc(-c2cc3cn[nH]c3cc2NC(=O)c2csc(-c3cc(C(F)(F)F)n[nH]3)n2)c1] | 497.1 | 496.1 | 4.75 | (R)-N-(5-(3-(1- aminoethyl)phenyl)- 1H-indazol-6-yl)-2-(3- (trifluoromethyl)-1H- pyrazol-5-yl)thiazole- 4-carboxamide |
| 1-11 | 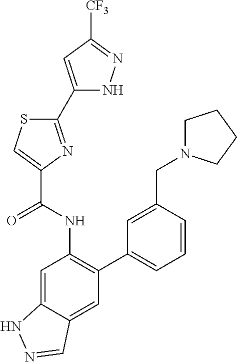 [SMILES: O=C(Nc1cc2[nH]ncc2cc1-c1cccc(CN2CCCC2)c1)c1csc(-c2cc(C(F)(F)F)n[nH]2)n1] | 537.1 | 538.4 | 5.16 | N-(5-(3-(pyrrolidin-1- ylmethyl)phenyl)-1H- indazol-6-yl)-2-(3- (trifluoromethyl)-1H- pyrazol-5-yl)thiazole- 4-carboxamide |
| 1-12 | 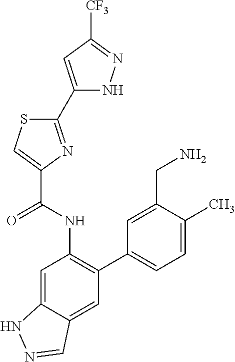 [SMILES: Cc1ccc(-c2cc3cn[nH]c3cc2NC(=O)c2csc(-c3cc(C(F)(F)F)n[nH]3)n2)cc1CN] | 497.1 | 498.7 | 5.19 | N-(5-(3- (aminomethyl)-4- methylphenyl)-1H- indazol-6-yl)-2-(3- (trifluoromethyl)-1H- pyrazol-5-yl)thiazole- 4-carboxamide |
| 1-13 | 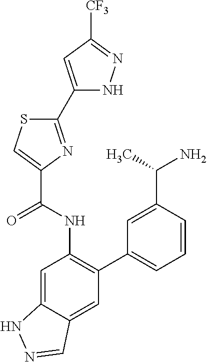 [SMILES: C[C@H](N)c1cccc(-c2cc3cn[nH]c3cc2NC(=O)c2csc(-c3cc(C(F)(F)F)n[nH]3)n2)c1] | 497.1 | 498.6 | 5.93 | (S)-N-(5-(3-(1- aminoethyl)phenyl)- 1H-indazol-6-yl)-2-(3- (trifluoromethyl)-1H- pyrazol-5-yl)thiazole- 4-carboxamide |
| 1-14 | 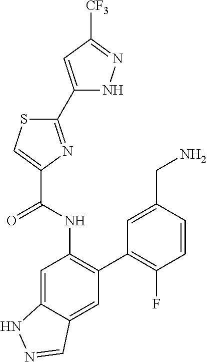 [SMILES: NCc1ccc(F)c(-c2cc3cn[nH]c3cc2NC(=O)c2csc(-c3cc(C(F)(F)F)n[nH]3)n2)c1] | 501.1 | 502.2 | 5.88 | N-(5-(5- (aminomethyl)-2- fluorophenyl)-1H- indazol-6-yl)-2-(3- (trifluoromethyl)-1H- pyrazol-5-yl)thiazole- 4-carboxamide |
| 1-15 | 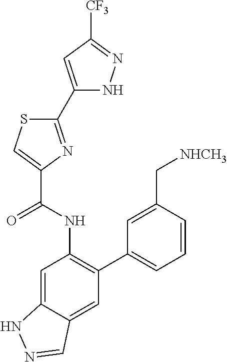 [SMILES: CNCc1cccc(-c2cc3cn[nH]c3cc2NC(=O)c2csc(-c3cc(C(F)(F)F)n[nH]3)n2)c1] | 497.1 | 498.6 | 5.97 | N-(5-(3 - ((methylamino)methyl) phenyl)-1H-indazol- 6-yl)-2-(3- (trifluoromethyl)-1H- pyrazol-5-yl)thiazole- 4-carboxamide |
| 1-16 | 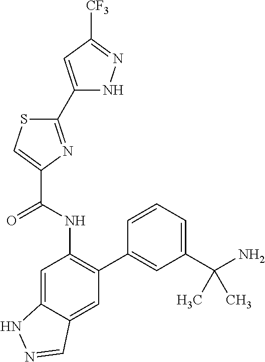 [SMILES: CC(C)(N)c1cccc(-c2cc3cn[nH]c3cc2NC(=O)c2csc(-c3cc(C(F)(F)F)n[nH]3)n2)c1] | 511.1 | 512.7 | 6.00 | N-(5-(3-(2- aminopropan-2- yl)phenyl)-1H- indazol-6-yl)-2-(3- (trifluoromethyl)-1H- pyrazol-5-yl)thiazole- 4-carboxamide |
| 1-17 | 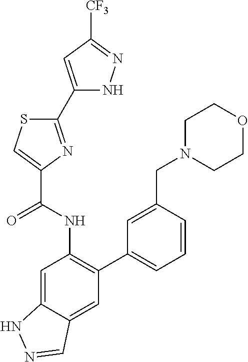 [SMILES: O=C(Nc1cc2[nH]ncc2cc1-c1cccc(CN2CCOCC2)c1)c1csc(-c2cc(C(F)(F)F)n[nH]2)n1] | 553.1 | 554.4 | 6.04 | N-(5-(3- (morpholinomethyl) phenyl)-1H-indazol-6- yl)-2-(3- (trifluoromethyl)-1H- pyrazol-5-yl)thiazole- 4-carboxamide |
| 1-18 | 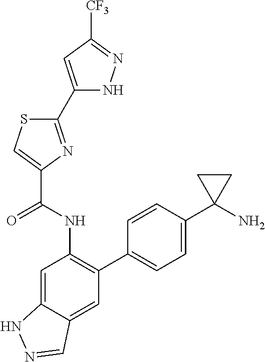 [SMILES: NC1(c2ccc(-c3cc4cn[nH]c4cc3NC(=O)c3csc(-c4cc(C(F)(F)F)n[nH]4)n3)cc2)CC1] | 509.1 | 510.5 | 5.93 | N-(5-(4-(1- aminocyclopropyl) phenyl)-1H-indazol-6-yl)- 2-(3-(trifluoromethyl)- 1H-pyrazol-5- yl)thiazole-4- carboxamide |
| 1-19 | 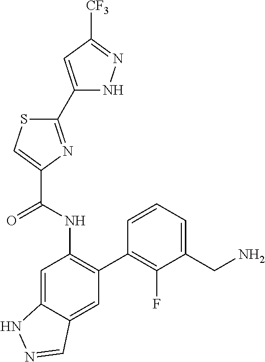 [SMILES: NCc1cccc(-c2cc3cn[nH]c3cc2NC(=O)c2csc(-c3cc(C(F)(F)F)n[nH]3)n2)c1F] | 501.1 | 500.2 | 5.83 | N-(5-(3- (aminomethyl)-2- fluorophenyl)-1H- indazol-6-yl)-2-(3- (trifluoromethyl)-1H- pyrazol-5-yl)thiazole- 4-carboxamide |
| 1-20 | 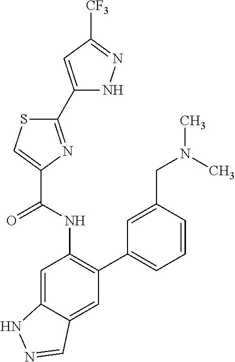 [SMILES: CN(C)Cc1cccc(-c2cc3cn[nH]c3cc2NC(=O)c2csc(-c3cc(C(F)(F)F)n[nH]3)n2)c1] | 511.1 | 512.7 | 6.07 | N-(5-(3- ((dimethylamino) methyl)phenyl)-1H- indazol-6-yl)-2-(3- (trifluoromethyl)-1H- pyrazol-5-yl)thiazole- 4-carboxamide |
| 1-21 | 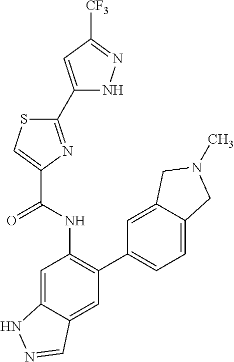 [SMILES: CN1Cc2ccc(-c3cc4cn[nH]c4cc3NC(=O)c3csc(-c4cc(C(F)(F)F)n[nH]4)n3)cc2C1] | 509.1 | 510.6 | 5.83 | N-(5-(2- methylisoindolin-5- yl)-1H-indazol-6-yl)- 2-(3-(trifluoromethyl)- 1H-pyrazol-5- yl)thiazole-4- carboxamide |
| 1-22 | 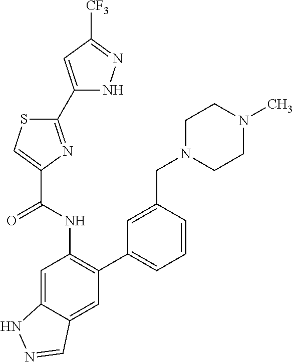 [SMILES: CN1CCN(Cc2cccc(-c3cc4cn[nH]c4cc3NC(=O)c3csc(-c4cc(C(F)(F)F)n[nH]4)n3)c2)CC1] | 566.1 | 567.5 | 5.73 | N-(5-(3-((4- methylpiperazin-1- yl)methyl)phenyl)-1H- indazol-6-yl)-2-(3- (trifluoromethyl)-1H- pyrazol-5-yl)thiazole- 4-carboxamide |
| 1-23 | 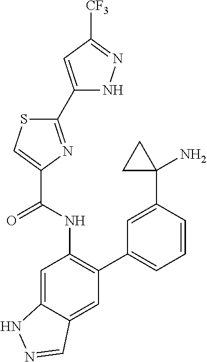 [SMILES: NC1(c2cccc(-c3cc4cn[nH]c4cc3NC(=O)c3csc(-c4cc(C(F)(F)F)n[nH]4)n3)c2)CC1] | 509.1 | 510.3 | 6.00 | N-(5-(3-(1- aminocyclopropyl) phenyl)-1H-indazol-6-yl)- 2-(3-(trifluoromethyl)- 1H-pyrazol-5- yl)thiazole-4- carboxamide |
| 1-24 | 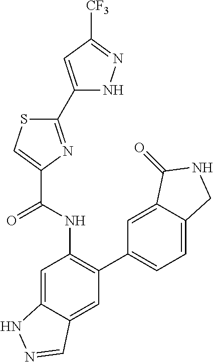 [SMILES: O=C(Nc1cc2[nH]ncc2cc1-c1ccc2c(c1)C(=O)NC2)c1csc(-c2cc(C(F)(F)F)n[nH]2)n1] | 509.0 | 510.5 | 6.65 | N-(5-(3- oxoisoindolin-5-yl)- 1H-indazol-6-yl)-2-(3- (trifluoromethyl)-1H- pyrazol-5-yl)thiazole- 4-carboxamide |
| 1-25 | 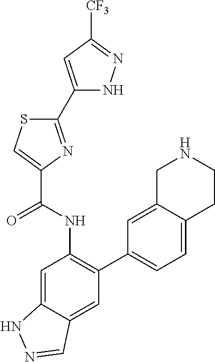 [SMILES: O=C(Nc1cc2[nH]ncc2cc1-c1ccc2c(c1)CNCC2)c1csc(-c2cc(C(F)(F)F)n[nH]2)n1] | 509.1 | 510.4 | 5.89 | N-(5-(1,2,3,4- tetrahydroisoquinolin- 7-yl)-1H-indazol-6- yl)-2-(3- (trifluoromethyl)-1H- pyrazol-5-yl)thiazole- 4-carboxamide |
| 1-26 | 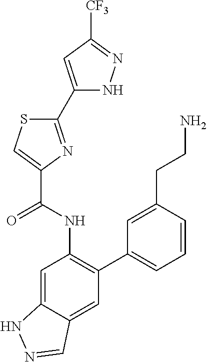 [SMILES: NCCc1cccc(-c2cc3cn[nH]c3cc2NC(=O)c2csc(-c3cc(C(F)(F)F)n[nH]3)n2)c1] | 497.1 | 496.2 | 6.01 | N-(5-(3-(2- aminoethyl)phenyl)- 1H-indazol-6-yl)-2-(3- (trifluoromethyl)-1H- pyrazol-5-yl)thiazole- 4-carboxamide |
| 1-27 | 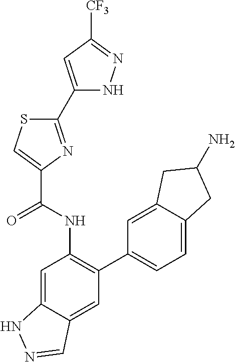 [SMILES: NC1Cc2ccc(-c3cc4cn[nH]c4cc3NC(=O)c3csc(-c4cc(C(F)(F)F)n[nH]4)n3)cc2C1] | 509.1 | 508.2 | 5.86 | N-(5-(2-amino-2,3- dihydro-1H-inden-5- yl)-1H-indazol-6-yl)- 2-(3-(trifluoromethyl)- 1H-pyrazol-5- yl)thiazole-4- carboxamide |
| 1-28 | 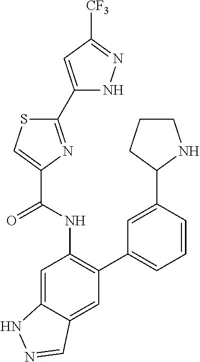 [SMILES: O=C(Nc1cc2[nH]ncc2cc1-c1cccc(C2CCCN2)c1)c1csc(-c2cc(C(F)(F)F)n[nH]2)n1] | 523.1 | 524.9 | 6.14 | N-(5-(3-(pyrrolidin-2- yl)phenyl)-1H- indazol-6-yl)-2-(3- (trifluoromethyl)-1H- pyrazol-5-yl)thiazole- 4-carboxamide |
| 1-29 | 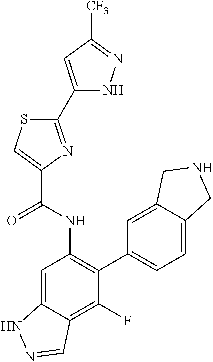 [SMILES: O=C(Nc1cc2[nH]ncc2c(F)c1-c1ccc2c(c1)CNC2)c1csc(-c2cc(C(F)(F)F)n[nH]2)n1] | 513.1 | 512.1 | 5.05 | N-(4-fluoro-5- (isoindolin-5-yl)-1H- indazol-6-yl)-2-(3- (trifluoromethyl)-1H- pyrazol-5-yl)thiazole- 4-carboxamide |

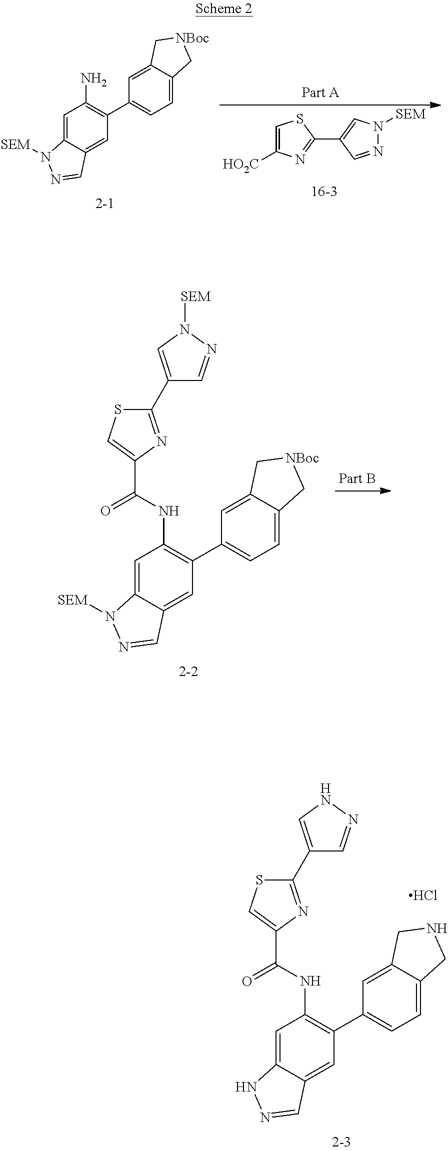

### N-(5-(isoindolin-5-yl)-1H-indazol-6-yl)-2-(1H-pyrazol-4-yl)thiazole-4-carboxamide Hydrochloride (2-3)

### N-(5-(isoindolin-5-yl)-1H-indazol-6-yl)-2-(1H-pyrazol-4-yl)thiazole-4-carboxamide Hydrochloride (2-2)

Part A: A mixture of 16-3 (54 mg, 0.166 mmol), tert-butyl 5-(6-amino-1-((2-(trimethylsilyl)ethoxy)methyl)-1H-indazol-5-yl)isoindoline-2-carboxylate (2-1) (96 mg, 0.20 mmol), HATU (95 mg, 0.249 mmol), and DIPEA (64 mg, 0.498 mmol) in DMF (1 mL) was stirred at room temperature 3.5 hours. The mixture was diluted with ethyl acetate, washed with saturated aqueous sodium bicarbonate and brine, dried over sodium sulfate, filtered, concentrated under vacuum, and purified by silica gel chromatography to afford tert-butyl 5-(1-((2-(trimethylsilyl)ethoxy)methyl)-6-(2-(1-((2-(trimethylsilyl)ethoxy)methyl)-1H-pyrazol-4-yl)thiazole-4-carboxamido)-1H-indazol-5-yl)isoindoline-2-carboxylate (2-2).

### N-(5-(isoindolin-5-yl)-1H-indazol-6-yl)-2-(1H-pyrazol-4-yl)thiazole-4-carboxamide Hydrochloride (2-3)

Part B: A mixture of tert-butyl 5-(1-((2-(trimethylsilyl)ethoxy)methyl)-6-(2-(1-((2-(trimethylsilyl)ethoxy)methyl)-1H-pyrazol-4-yl)thiazole-4-carboxamido)-1H-indazol-5-yl)isoindoline-2-carboxylate (2-2) (66 mg, 0.0837 mmol) and TFA (4 mL) in dichloromethane (2 mL) and water (2 drops) was stirred at room temperature for 6 hours. The reaction was concentrated and purified by prep-HPLC to afford N-(5-(isoindolin-5-yl)-1H-indazol-6-yl)-2-(1H-pyrazol-4-yl)thiazole-4-carboxamide hydrochloride (2-3). ¹H NMR (400 MHz, DMSO d₆) δ 13.14 (bs, 1H); 9.77 (s, 1H); 9.67 (bs, 2H); 8.67 (s, 1H); 8.29 (s, 1H); 8.08 (s, 1H); 7.96 (bs, 2H); 7.69 (s, 1H); 7.66-7.557 (m, 3H); 4.68-4.65 (m, 2H); 4.59-4.56 (m, 2H). HPLC t_{R}=5.27 min (UV_{254 nm}). Mass calculated for formula C₂₂H₁₇N₇OS 427.11; observed MH⁺ (ESI MS) 428.3 (m/z).

The compounds in Table 2 were prepared according to the Reaction Schemes and Scheme 2. The free base is shown.

TABLE 2

| | | | MS | | |
|---|---|---|---|---|---|
| | | | MH⁺/MH⁻ | HPLC | |
| Cmp | Structure | MW | m/z | t_{R} | Name |
| 2-5 | 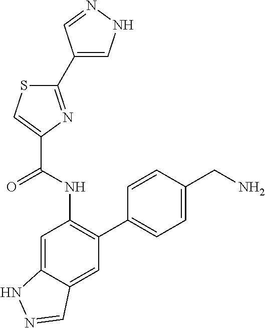 [SMILES: NCc1ccc(-c2cc3cn[nH]c3cc2NC(=O)c2csc(-c3cn[nH]c3)n2)cc1] | 415.1 | 416.7 | 4.25 | N-(5-(4- (aminomethyl)phenyl)- 1H-indazol-6-yl)-2- (1H-pyrazol-4- yl)thiazole-4- carboxamide |
| 2-6 | 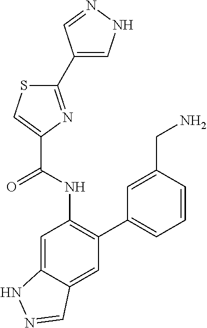 [SMILES: NCc1cccc(-c2cc3cn[nH]c3cc2NC(=O)c2csc(-c3cn[nH]c3)n2)c1] | 415.1 | 415.2 | 5.17 | N-(5-(3- (aminomethyl)phenyl)- 1H-indazol-6-yl)-2- (1H-pyrazol-4- yl)thiazole-4- carboxamide |

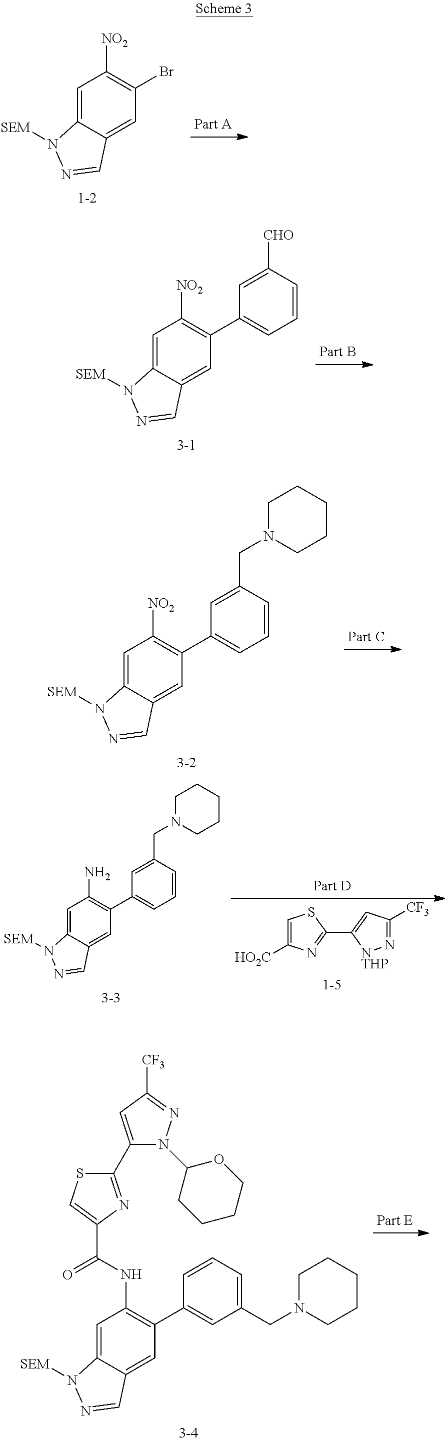

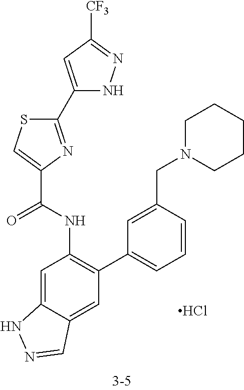 [SMILES: O=C(Nc1cc2[nH]ncc2cc1-c1cccc(CN2CCCCC2)c1)c1csc(-c2cc(C(F)(F)F)n[nH]2)n1 ; Cl]

### N-(5-(3-(piperidin-1-ylmethyl)phenyl)-1H-indazol-6-yl)-2-(3-(trifluoromethyl)-1H-pyrazol-5-yl)thiazole-4-carboxamide Hydrochloride (3-5)

### 3-(6-nitro-1-((2-(trimethylsilyl)ethoxy)methyl)-1H-indazol-5-yl)benzaldehyde (3-1)

Part A: A mixture of 5-bromo-6-nitro-1-((2-(trimethylsilyl)ethoxy)methyl)-1H-indazole (1-2) (1.20 g, 31 mmol), 3-formylbenzeneboronic acid (530 mg, 3.2 mmol), PdCl₂dppf.CH₂Cl₂ (260 mg, 0.32 mmol) and K₂CO₃ (1.33 g, 9.6 mmol) in DMF (6 mL) was stirred at 85° C. under a nitrogen atmosphere for 18 hours. The mixture was allowed to cool to room temperature, diluted with ethyl acetate, washed with water and brine, dried over sodium sulfate, filtered, concentrated under vacuum, and purified by silica gel chromatography to afford 3-(6-nitro-1-((2-(trimethylsilyl)ethoxy)methyl)-1H-indazol-5-yl)benzaldehyde (3-1).

### 6-nitro-5-(3-(piperidin-1-ylmethyl)phenyl)-1-((2-(trimethylsilyl)ethoxy)methyl)-1H-indazole (3-2)

Part B: A mixture of 3-(6-nitro-1-((2-(trimethylsilyl)ethoxy)methyl)-1H-indazol-5-yl)benzaldehyde (3-1) (100 mg, 0.25 mmol), piperidine (43 mg, 0.5 mmol), and titanium (IV) isopropoxide (142 mg, 0.5 mmol), in ethanol (2 mL) was stirred at room temperature for 8 hours. NaBH₄ (14 mg, 0.37 mmol) was added and stirred at room temperature until the reaction was deemed complete as judged by TLC analysis. The mixture was quenched with NH₃.H₂O (2N), diluted with dichloromethane, filtered through Celite, washed with brine, dried over sodium sulfate, filtered, concentrated under vacuum, and purified by silica gel chromatography to afford 6-nitro-5-(3-(piperidin-1-ylmethyl)phenyl)-1-((2-(trimethylsilyl)ethoxy)methyl)-1H-indazole (3-2).

### 5-(3-(piperidin-1-ylmethyl)phenyl)-1-((2-(trimethylsilyl)ethoxy)methyl)-1H-indazol-6-amine (3-3)

Part C: A mixture of 6-nitro-5-(3-(piperidin-1-ylmethyl)phenyl)-1-((2-(trimethylsilyl)ethoxy)methyl)-1H-indazole (3-2) (110 mg, 024 mmol) and Pd/C (10% Pd, 80 mg, 0.075 mmol) in ethyl acetate (3.0 mL) was stirred under an atmosphere of hydrogen gas at room temperature for 18 hours. The mixture was then filtered through a pad of Celite and concentrated under vacuum to afford 5-(3-(piperidin-1-ylmethyl)phenyl)-1-((2-(trimethylsilyl)ethoxy)methyl)-1H-indazol-6-amine (3-3).

### N-(5-(3-(piperidin-1-ylmethyl)phenyl)-1-((2-(trimethylsilyl)ethoxy)methyl)-1H-indazol-6-yl)-2-(1-(tetrahydro-2H-pyran-2-yl)-3-(trifluoromethyl)-1H-pyrazol-5-yl)thiazole-4-carboxamide (3-4)

Part D: A mixture of 2-(1-(tetrahydro-2H-pyran-2-yl)-3-(trifluoromethyl)-1H-pyrazol-5-yl)thiazole-4-carboxylic acid (1-5) (38 mg, 0.11 mmol), 5-(3-(piperidin-1-ylmethyl)phenyl)-1-((2-(trimethylsilyl)ethoxy)methyl)-1H-indazol-6-amine (3-3)

(48 mg, 0.11 mmol), HATU (63 mg, 0.17 mmol), and DIPEA (43 mg, 0.33 mmol) in DMF (3 mL) was stirred at room temperature for 18 hours. The mixture was diluted with ethyl acetate, washed with saturated aqueous sodium bicarbonate and brine, dried over sodium sulfate, filtered, concentrated under vacuum, and purified by silica gel chromatography to afford compound N-(5-(3-(piperidin-1-ylmethyl)phenyl)-1-((2-(trimethylsilyl)ethoxy)methyl)-1H-indazol-6-yl)-2-(1-(tetrahydro-2H-pyran-2-yl)-3-(trifluoromethyl)-1H-pyrazol-5-yl)thiazole-4-carboxamide (3-4).

### N-(5-(3-(piperidin-1-ylmethyl)phenyl)-1H-indazol-6-yl)-2-(3-(trifluoromethyl)-1H-pyrazol-5-yl)thiazole-4-carboxamide Hydrochloride (3-5)

Part E: A mixture of N-(5-(3-(piperidin-1-ylmethyl)phenyl)-1-((2-(trimethylsilyl)ethoxy)methyl)-1H-indazol-6-yl)-2-(1-(tetrahydro-2H-pyran-2-yl)-3-(trifluoromethyl)-1H-pyrazol-5-yl)thiazole-4-carboxamide (3-4) (66 mg, 0.0862 mmol) and TFA (4 mL) in dichloromethane (2 mL) and water (2 drops) was stirred at 60° C. for 18 hours. The reaction was allowed to cool to room temperature, concentrated under vacuum, and purified by prep-HPLC to afford N-(5-(3-(piperidin-1-ylmethyl)phenyl)-1H-indazol-6-yl)-2-(3-(trifluoromethyl)-1H-pyrazol-5-yl)thiazole-4-carboxamide hydrochloride (3-5). ¹H NMR (400 MHz, DMSO d₆) δ 8.51 (bs, 1H); 8.34 (bs, 1H); 8.09 (s, 1H); 7.79 (s, 1H); 7.74-7.68 (m, 2H); 7.64 (bs, 1H); 7.59-7.57 (m, 1H); 6.93 (bs, 1H); 4.30 (s, 2H); 3.41-3.38 (m, 2H); 2.92-2.86 (m, 2H); 1.81-1.74 (m, 3H); 1.66-1.56 (m, 2H); 1.43-1.32 (m, 1H). HPLC t_{R}=6.25 min (UV_{254 nm}). Mass calculated for formula C₂₇H₂₄F₃N₇OS 551.17; observed MH⁺ (ESI MS) 552.7 (m/z).

The compounds in Table 3 were prepared according to the Reaction Schemes and Scheme 3. The free base is shown.

TABLE 3

| | | | MS | | |
|---|---|---|---|---|---|
| | | | MH⁺/MH⁻ | HPLC | |
| Cmp | Structure | MW | m/z | t_{R} | Name |
| 3-6 | 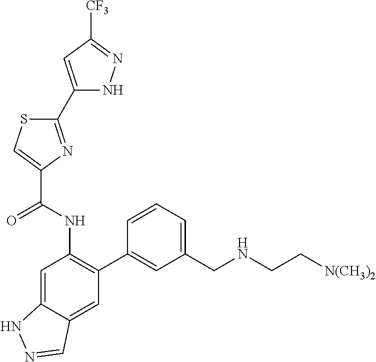 [SMILES: CN(C)CCNCc1cccc(-c2cc3cn[nH]c3cc2NC(=O)c2csc(-c3cc(C(F)(F)F)n[nH]3)n2)c1] | 554.1 | 553.3 | 5.55 | N-(5-(3-((2- (dimethylamino) ethylamino)methyl) phenyl)-1H-indazol-6- yl)-2-(3- (trifluoromethyl)- 1H-pyrazol-5- yl)thiazole-4- carboxamide |
| 3-7 | 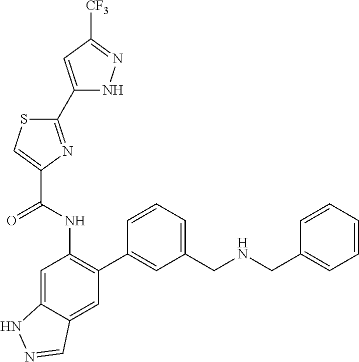 [SMILES: O=C(Nc1cc2[nH]ncc2cc1-c1cccc(CNCc2ccccc2)c1)c1csc(-c2cc(C(F)(F)F)n[nH]2)n1] | 573.1 | 574.6 | 6.39 | N-(5-(3- ((benzylamino) methyl)phenyl)-1H- indazol-6-yl)-2-(3- (trifluoromethyl)- 1H-pyrazol-5- yl)thiazole-4- carboxamide |
| 3-8 | 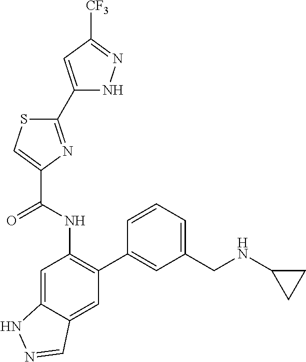 [SMILES: O=C(Nc1cc2[nH]ncc2cc1-c1cccc(CNC2CC2)c1)c1csc(-c2cc(C(F)(F)F)n[nH]2)n1] | 523.1 | 524.3 | 6.21 | N-(5-(3- ((cyclopropylamino) methyl)phenyl)- 1H-indazol-6-yl)-2- (3- (trifluoromethyl)- 1H-pyrazol-5- yl)thiazole-4- carboxamide |
| 3-9 | 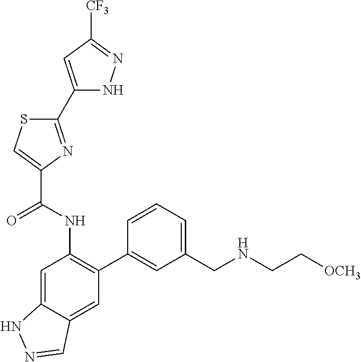 [SMILES: COCCNCc1cccc(-c2cc3cn[nH]c3cc2NC(=O)c2csc(-c3cc(C(F)(F)F)n[nH]3)n2)c1] | 541.1 | 542.4 | 6.17 | N-(5-(3-((2- methoxyethylamino) methyl)phenyl)- 1H-indazol-6-yl)-2- (3- (trifluoromethyl)- 1H-pyrazol-5- yl)thiazole-4- carboxamide |
| 3-10 | 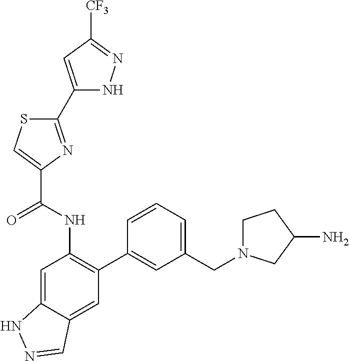 [SMILES: NC1CCN(Cc2cccc(-c3cc4cn[nH]c4cc3NC(=O)c3csc(-c4cc(C(F)(F)F)n[nH]4)n3)c2)C1] | 552.1 | 553.4 | 5.62 | N-(5-(3-((3- aminopyrrolidin-1- yl)methyl)phenyl)- 1H-indazol-6-yl)-2- (3- (trifluoromethyl)- 1H-pyrazol-5- yl)thiazole-4- carboxamide |
| 3-11 | 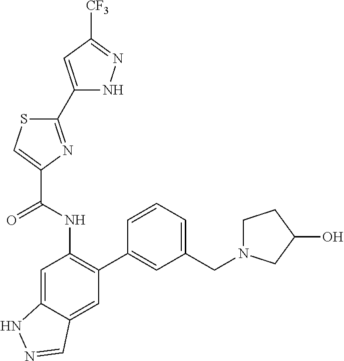 [SMILES: O=C(Nc1cc2[nH]ncc2cc1-c1cccc(CN2CCC(O)C2)c1)c1csc(-c2cc(C(F)(F)F)n[nH]2)n1] | 553.1 | 554.4 | 6.07 | N-(5-(3-((3- hydroxypyrrolidin- 1- yl)methyl)phenyl)- 1H-indazol-6-yl)-2- (3- (trifluoromethyl)- 1H-pyrazol-5- yl)thiazole-4- carboxamide |
| 3-12 | 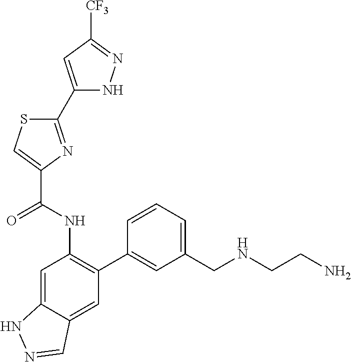 [SMILES: NCCNCc1cccc(-c2cc3cn[nH]c3cc2NC(=O)c2csc(-c3cc(C(F)(F)F)n[nH]3)n2)c1] | 526.1 | 527.3 | 5.28 | N-(5-(3-((2- aminoethylamino) methyl)phenyl)-1H- indazol-6-yl)-2-(3- (trifluoromethyl)- 1H-pyrazol-5- yl)thiazole-4- carboxamide |
| 3-13 | 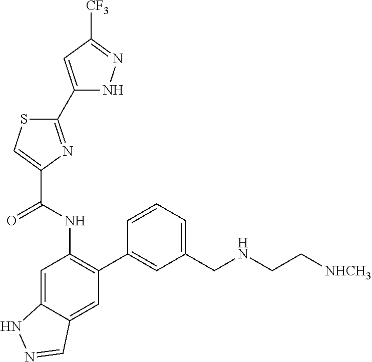 [SMILES: CNCCNCc1cccc(-c2cc3cn[nH]c3cc2NC(=O)c2csc(-c3cc(C(F)(F)F)n[nH]3)n2)c1] | 540.1 | 541.4 | 4.97 | N-(5-(3-((2- (methylamino)ethyl amino)methyl)phenyl)- 1H-indazol-6- yl)-2-(3- (trifluoromethyl)- 1H-pyrazol-5- yl)thiazole-4- carboxamide |
| 3-14 | 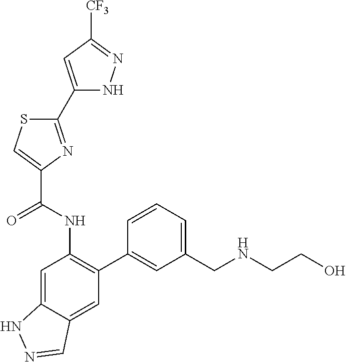 [SMILES: O=C(Nc1cc2[nH]ncc2cc1-c1cccc(CNCCO)c1)c1csc(-c2cc(C(F)(F)F)n[nH]2)n1] | 527.1 | 528.3 | 5.42 | N-(5-(3-((2- hydroxyethylamino) methyl)phenyl)-1H- indazol-6-yl)-2-(3- (trifluoromethyl)- 1H-pyrazol-5- yl)thiazole-4- carboxamide |

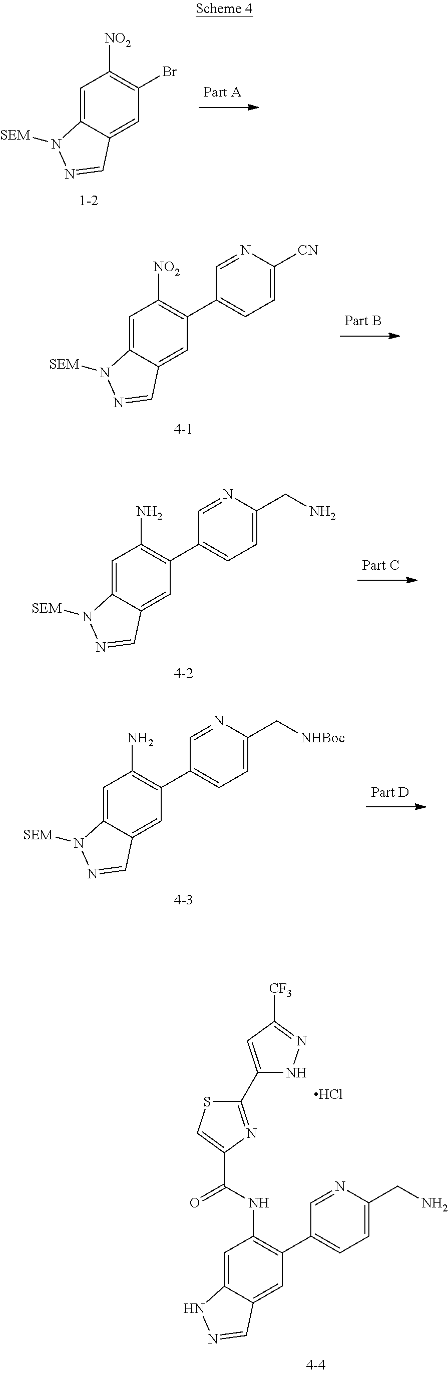 [SMILES: C[Si](C)(C)CCOCn1ncc2cc(Br)c([N+](=O)[O-])cc21 ; C[Si](C)(C)CCOCn1ncc2cc(-c3ccc(C#N)nc3)c([N+](=O)[O-])cc21 ; C[Si](C)(C)CCOCn1ncc2cc(-c3ccc(CN)nc3)c(N)cc21 ; CC(C)(C)OC(=O)NCc1ccc(-c2cc3cnn(COCC[Si](C)(C)C)c3cc2N)cn1 ; NCc1ccc(-c2cc3cn[nH]c3cc2NC(=O)c2csc(-c3cc(C(F)(F)F)n[nH]3)n2)cn1 ; Cl]

### N-(5-(6-(aminomethyl)pyridin-3-yl)-1H-indazol-6-yl)-2-(3-(trifluoromethyl)-1H-pyrazol-5-yl)thiazole-4-carboxamide Hydrochloride (4-4)

### 5-(6-nitro-1-((2-(trimethylsilyl)ethoxy)methyl)-1H-indazol-5-yl)picolinonitrile (4-1)

Part A: A mixture of 1-2 (150 mg, 0.403 mmol), 5-(4,4,5,5-tetramethyl-1,3,2-dioxaborolan-2-yl)picolinonitrile (111 mg, 0.484 mmol), PdCl₂dppf.CH₂Cl₂ (29 mg, 0.0403 mmol), and K₂CO₃ (111 mg, 0.806 mmol) in DMF (1.5 mL) was stirred under a nitrogen atmosphere at 85° C. for 16 hours. The mixture was allowed to cool to room temperature, diluted with ethyl acetate, washed with water and brine, dried over sodium sulfate, filtered, concentrated under vacuum, and purified by silica gel chromatography to afford 5-(4,4,5,5-tetramethyl-1,3,2-dioxaborolan-2-yl)picolinonitrile (4-1) as a yellow solid.

### 5-(6-(aminomethyl)pyridin-3-yl)-1-((2-(trimethylsilyl)ethoxy)methyl)-1H-indazol-6-amine (4-2)

Part B: A mixture of 4-1 (134 mg, 0.339 mmol) and Pd/C (10% Pd, 144 mg, 0.0677 mmol) in ethyl acetate (3.3 mL) and acetic acid (0.3 mL) was stirred under a hydrogen atmosphere at room temperature for 5 hours. The mixture was filtered through a pad of Celite and concentrated under vacuum to afford 5-(6-(aminomethyl)pyridin-3-yl)-1-((2-(trimethylsilyl)ethoxy)methyl)-1H-indazol-6-amine (4-2) as a brown oil.

### tert-butyl (5-(6-amino-1-((2-(trimethylsilyl)ethoxy)methyl)-1H-indazol-5-yl)pyridin-2-yl)methylcarbamate (4-3)

Part C: A mixture of 4-3 (135 mg, 0.314 mmol), Boc₂O (72 mg, 0.33 mmol) and TEA (38 mg, 0.377 mmol) in dichloromethane (1.5 mL) was stirred at room temperature for 22 hours. The reaction was diluted with ethyl acetate, washed with saturated aqueous ammonium chloride and brine, dried over sodium sulfate, filtered, concentrated under vacuum and purified by silica gel chromatography to afford tert-butyl (5-(6-amino-1-((2-(trimethylsilyl)ethoxy)methyl)-1H-indazol-5-yl)pyridin-2-yl)methylcarbamate (4-3) as a brown oil.

### N-(5-(6-(aminomethyl)pyridin-3-yl)-1H-indazol-6-yl)-2-(3-(trifluoromethyl)-1H-pyrazol-5-yl)thiazole-4-carboxamide Hydrochloride (4-4)

Part D: A mixture of 4-3 (30 mg, 0.0639 mmol), 2-(1-(tetrahydro-2H-pyran-2-yl)-3-(trifluoromethyl)-1H-pyrazol-5-yl)thiazole-4-carboxylic acid (18 mg, 0.0532 mmol), HATU (30 mg, 0.0798 mmol), and DIPEA (21 mg, 0.17 mmol) in DMF (1 mL) was stirred at room temperature for 16 hours. The reaction was diluted with ethyl acetate, washed with water and brine, dried over sodium sulfate, filtered, concentrated under vacuum, and purified by silica gel chromatography to give a white solid. The solid was stirred with HCl (4N in dioxane, 1 mL) in methanol (1 mL) and water (1 drop) at 40° C. for 14 hours, then 50° C. for 24 hours. The reaction was allowed to cool to room temperature, concentrated under vacuum, and purified by prep-HPLC to afford 4-4 as a white solid. ¹H NMR (400 MHz, DMSO d₆) δ 14.76 (bs, 1H); 13.24 (bs, 1H); 9.95 (s, 1H); 8.78 (bs, 1H); 8.50 (s, 1H); 8.42-8.32 (m, 3H); 8.22-8.12 (m, 1H); 8.10-8.09 (m, 1H); 7.83 (s, 1H); 7.68-7.59 (m, 1H); 7.27-7.15 (M, 1H); 4.20 (bs, 2H). HPLC t_{R}=4.30 min (UV_{254 nm}). Mass calculated for formula C₂₁H₁₅F₃N₈OS 484.11; observed MH⁺ (ESI MS) 485.5 (m/z).

The compounds in Table 4 were prepared according to the Reaction Schemes and Scheme 4. The free base is shown.

TABLE 4

| | | | MS | | |
|---|---|---|---|---|---|
| | | | MH⁺/MH⁻ | HPLC | |
| Cmp | Structure | MW | m/z | t_{R} | Name |
| 4-5 | 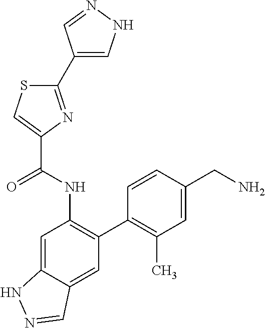 [SMILES: Cc1cc(CN)ccc1-c1cc2cn[nH]c2cc1NC(=O)c1csc(-c2cn[nH]c2)n1] | 429.1 | 431.4 | 4.38 | N-(5-(4- (aminomethyl)-2- methylphenyl)-1H- indazol-6-yl)-2-(1H- pyrazol-4-yl)thiazole- 4-carboxamide |
| 4-6 | 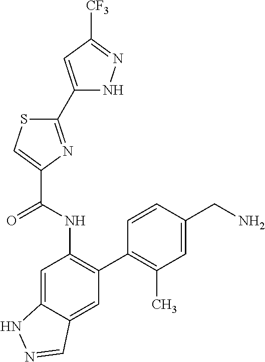 [SMILES: Cc1cc(CN)ccc1-c1cc2cn[nH]c2cc1NC(=O)c1csc(-c2cc(C(F)(F)F)n[nH]2)n1] | 497.1 | 496.8 | 4.97 | N-(5-(4- (aminomethyl)-2- methylphenyl)-1H- indazol-6-yl)-2-(3- (trifluoromethyl)-1H- pyrazol-5-yl)thiazole- 4-carboxamide |
| 4-7 | 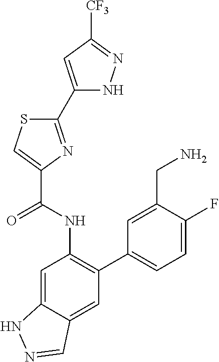 [SMILES: NCc1cc(-c2cc3cn[nH]c3cc2NC(=O)c2csc(-c3cc(C(F)(F)F)n[nH]3)n2)ccc1F] | 501.1 | 502.1 | 4.91 | N-(5-(3- (aminomethyl)-4- fluorophenyl)-1H- indazol-6-yl)-2-(3- (trifluoromethyl)-1H- pyrazol-5-yl)thiazole- 4-carboxamide |

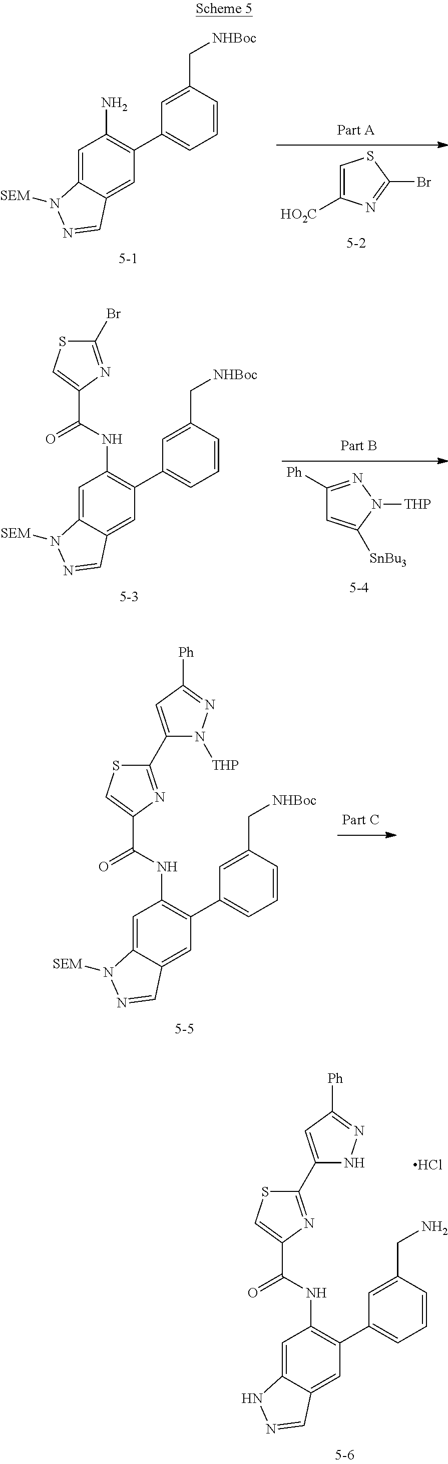

### N-(5-(3-(aminomethyl)phenyl)-1H-indazol-6-yl)-2-(3-phenyl-1H-pyrazol-5-yl)thiazole-4-carboxamide Hydrochloride (5-6)

### tert-butyl 3-(6-(2-bromothiazole-4-carboxamido)-1-((2-(trimethylsilyl)ethoxy)methyl)-1H-indazol-5-yl)benzylcarbamate (5-3)

Part A: A mixture of tert-butyl 3-(6-amino-1-((2-(trimethylsilyl)ethoxy)methyl)-1H-indazol-5-yl)benzylcarbamate (5-1) (352 mg, 0.752 mmol), 2-Bromo-4-thiazolecarboxylic acid (5-2) (156 mg, 0.902 mmol), HATU (429 mg, 1.13 mmol), and DIPEA (292 mg, 2.26 mmol) in DMF (4 mL) was stirred at room temperature for 18 hours. The mixture was diluted with ethyl acetate, washed with saturated aqueous sodium bicarbonate and brine, dried over sodium sulfate, filtered, concentrated under vacuum, and purified by silica gel chromatography to afford tert-butyl 3-(6-(2-bromothiazole-4-carboxamido)-1-((2-(trimethylsilyl)ethoxy)methyl)-1H-indazol-5-yl)benzylcarbamate (5-3).

### tert-butyl 3-(6-(2-(3-phenyl-1-(tetrahydro-2H-pyran-2-yl)-1H-pyrazol-5-yl)thiazole-4-carboxamido)-1-((2-(trimethylsilyl)ethoxy)methyl)-1H-indazol-5-yl)benzylcarbamate (5-5)

Part B: A mixture of tert-butyl 3-(6-(2-bromothiazole-4-carboxamido)-1-((2-(trimethylsilyl)ethoxy)methyl)-1H-indazol-5-yl)benzylcarbamate (5-3) (120 mg, 0.182 mmol), 3-phenyl-1-(tetrahydro-2H-pyran-2-yl)-5-(tributylstannyl)-1H-pyrazole (5-4) (188 mg, 0.364 mmol), Pd(PPh₃)₄ (42 mg, 0.0364) in 1,4-dioxane (3 mL) was stirred at 110° C. for 72 h. The mixture was allowed to cool to room temperature, diluted with ethyl acetate, washed with water and brine, dried over sodium sulfate, filtered, concentrated under vacuum, and purified by silica gel chromatography to afford tert-butyl 3-(6-(2-(3-phenyl-1-(tetrahydro-2H-pyran-2-yl)-1H-pyrazol-5-yl)thiazole-4-carboxamido)-1-((2-(trimethylsilyl)ethoxy)methyl)-1H-indazol-5-yl)benzylcarbamate (5-5).

### N-(5-(3-(aminomethyl)phenyl)-1H-indazol-6-yl)-2-(3-phenyl-1H-pyrazol-5-yl)thiazole-4-carboxamide Hydrochloride (5-6)

Part C: A mixture of tert-butyl 3-(6-(2-(3-phenyl-1-(tetrahydro-2H-pyran-2-yl)-1H-pyrazol-5-yl)thiazole-4-carboxamido)-1-((2-(trimethylsilyl)ethoxy)methyl)-1H-indazol-5-yl)benzylcarbamate (5-5) (150 mg, 0.182 mmol) and HCl (4N in dioxane, 4 mL, 16 mmol) in methanol (2 mL) and water (1 mL) was stirred at 60° C. for 16 hours. The reaction was allowed to cool to room temperature, concentrated under vacuum, and purified by prep-HPLC to afford N-(5-(3-(aminomethyl)phenyl)-1H-indazol-6-yl)-2-(3-phenyl-1H-pyrazol-5-yl)thiazole-4-carboxamide hydrochloride (5-6). ¹H NMR (400 MHz, DMSO d₆) δ 13.93 (bs, 1H); 13.18 (bs, 1H); 9.86 (s, 1H); 8.78 (s, 1H); 8.44 (s, 1H); 8.30 (bs, 3H); 8.12 (s, 1H); 8.78-8.48 (m, 10H); 6.74 (s, 1H); 4.13-4.10 (m, 2H). HPLC t_{R}=6.09 min (UV_{254 nm}). Mass calculated for formula C₂₇H₂₁N₇OS 491.15; observed MH⁺ (ESI MS) 492.3 (m/z).

The compound in Table 5 was prepared according to the Reaction Schemes and Scheme 5. The free base is shown.

TABLE 5

| | | | MS | | |
|---|---|---|---|---|---|
| | | | MH⁺/MH⁻ | HPLC | |
| Cmp | Structure | MW | m/z | t_{R} | Name |
| 5-7 | 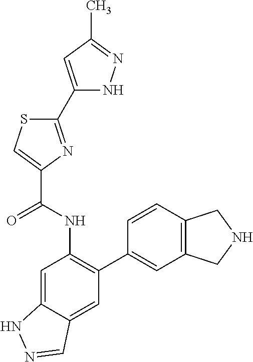 [SMILES: Cc1cc(-c2nc(C(=O)Nc3cc4[nH]ncc4cc3-c3ccc4c(c3)CNC4)cs2)[nH]n1] | 441.1 | 442.4 | 5.47 | N-(5-(isoindolin- 5-yl)-1H- indazol-6-yl)-2- (3-methyl-1H- pyrazol-5- yl)thiazole-4- carboxamide |

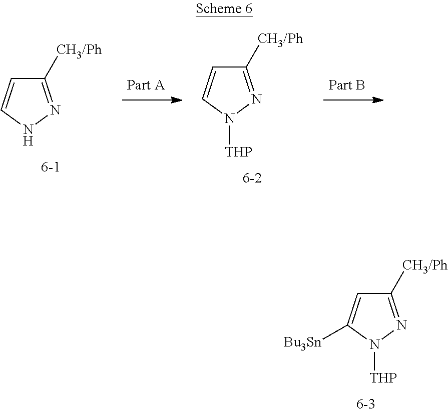

### Compound 6-3

Part A: A mixture of 6-1 (1 equiv), 3,4-dihydro-2H-pyran (1.5 equiv), and TFA (5%) in toluene (1 M) was stirred at room temperature until the reaction was deemed complete as judged by TLC analysis. The mixture was diluted with ethyl acetate, washed with saturated aqueous sodium bicarbonate and brine, dried over sodium sulfate, filtered, concentrated, and purified on silica gel to afford compound 6-2.

Part B: To a solution of compound 6-2 (1 equiv) in THF (1 M) was added n-BuLi (1 equiv) at −78° C. under nitrogen. The mixture was allowed to warm to −45° C., then stirred for 1 hour. The reaction mixture was cooled to −78° C. and tributyltinchloride (1.1 equiv) was added. The reaction mixture was allowed to warm to room temperature. The mixture was diluted with ethyl acetate, washed with water and brine, dried over sodium sulfate, filtered, concentrated under vacuum, and purified by silica gel chromatography to afford 6-3.

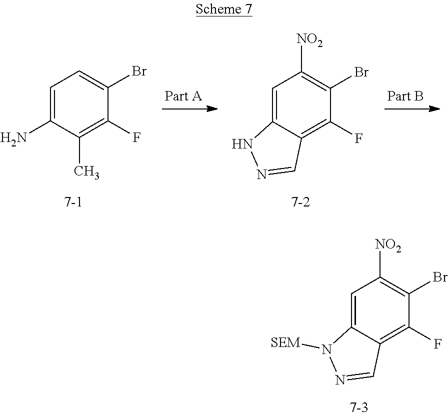

### 5-bromo-4-fluoro-6-nitro-1-((2-(trimethylsilyl)ethoxy)methyl)-1H-indazole (7-3)

### 5-bromo-4-fluoro-6-nitro-1H-indazole (7-2)

Part A: 4-bromo-3-fluoro-2-methylaniline (7-1) was converted to 5-bromo-4-fluoro-6-nitro-1H-indazole (7-2) in a similar manner to compound 1-1.¹

### 5-bromo-4-fluoro-6-nitro-1-((2-(trimethylsilyl)ethoxy)methyl)-1H-indazole (7-3)

Part B: 5-bromo-4-fluoro-6-nitro-1H-indazole (7-2) was converted to 5-bromo-4-fluoro-6-nitro-1-((2-(trimethylsilyl)ethoxy)methyl)-1H-indazole (7-3) in a similar manner to compound 1-2.

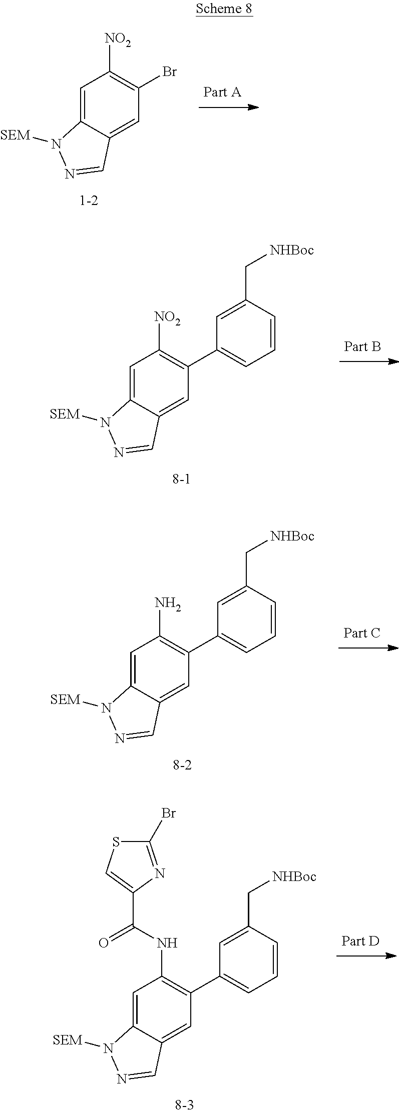 [SMILES: C[Si](C)(C)CCOCn1ncc2cc(Br)c([N+](=O)[O-])cc21 ; CC(C)(C)OC(=O)NCc1cccc(-c2cc3cnn(COCC[Si](C)(C)C)c3cc2[N+](=O)[O-])c1 ; CC(C)(C)OC(=O)NCc1cccc(-c2cc3cnn(COCC[Si](C)(C)C)c3cc2N)c1 ; CC(C)(C)OC(=O)NCc1cccc(-c2cc3cnn(COCC[Si](C)(C)C)c3cc2NC(=O)c2csc(Br)n2)c1]

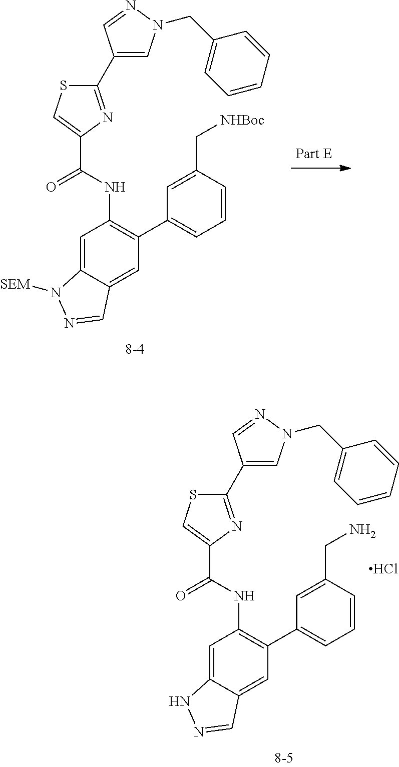 [SMILES: CC(C)(C)OC(=O)NCc1cccc(-c2cc3cnn(COCC[Si](C)(C)C)c3cc2NC(=O)c2csc(-c3cnn(Cc4ccccc4)c3)n2)c1 ; NCc1cccc(-c2cc3cn[nH]c3cc2NC(=O)c2csc(-c3cnn(Cc4ccccc4)c3)n2)c1 ; Cl]

### N-(5-(3-(aminomethyl)phenyl)-1H-indazol-6-yl)-2-(1-benzyl-1H-pyrazol-4-yl)thiazole-4-carboxamide Hydrochloride (8-5)

### tert-butyl 3-(6-nitro-1-((2-(trimethylsilyl)ethoxy)methyl)-1H-indazol-5-yl)benzylcarbamate (8-1)

Part A: A mixture of 5-bromo-6-nitro-1-((2-(trimethylsilyl)ethoxy)methyl)-1H-indazole (1-2) (400 mg, 1.07 mmol), tert-butyl 3-(4,4,5,5-tetramethyl-1,3,2-dioxaborolan-2-yl)benzylcarbamate (324 mg, 1.29 mmol), PdCl₂dppf.CH₂Cl₂ (87 mg, 0.107 mmol), and K₂CO₃ (296 mg, 2.14 mmol) in DMF (5 mL) was stirred at 85° C. for 19 hours under a nitrogen atmosphere. The reaction was allowed to cool to room temperature, diluted with ethyl acetate, washed with brine, dried over sodium sulfate, filtered, concentrated under vacuum, and purified by silica gel chromatography to afford tert-butyl 3-(6-nitro-1-((2-(trimethylsilyl)ethoxy)methyl)-1H-indazol-5-yl)benzylcarbamate (8-1).

### tert-butyl 3-(6-amino-1-((2-(trimethylsilyl)ethoxy)methyl)-1H-indazol-5-yl)benzylcarbamate (8-2)

Part B: A mixture of tert-butyl 3-(6-nitro-1-((2-(trimethylsilyl)ethoxy)methyl)-1H-indazol-5-yl)benzylcarbamate (8-1) (375 mg, 0.752 mmol) and Pd/C (10% Pd, 180 mg, 0.0752 mmol) in ethyl acetate (10 mL) was stirred under an atmosphere of hydrogen gas for 15 hours. The mixture was then filtered through a pad of Celite and concentrated to afford tert-butyl 3-(6-amino-1-((2-(trimethylsilyl)ethoxy)methyl)-1H-indazol-5-yl)benzylcarbamate (8-2).

### tert-butyl 3-(6-(2-bromothiazole-4-carboxamido)-1-((2-(trimethylsilyl)ethoxy)methyl)-1H-indazol-5-yl)benzylcarbamate (8-3)

Part C: A mixture of thiazolecarboxylic acid (156 mg, 0.75 mmol), tert-butyl 3-(6-amino-1-((2-(trimethylsilyl)ethoxy)methyl)-1H-indazol-5-yl)benzylcarbamate (8-2) (370 mg, 0.75 mmol), HATU (429 mg, 1.128 mmol), and DIPEA (292 mg, 2.256 mmol) in DMF (4 mL) was stirred at room temperature for 17 hours. The mixture was diluted with ethyl acetate, washed with brine, dried over sodium sulfate, filtered, concentrated under vacuum, and purified by silica gel chromatography to afford tert-butyl 3-(6-(2-bromothiazole-4-carboxamido)-1-((2-(trimethylsilyl)ethoxy)methyl)-1H-indazol-5-yl)benzylcarbamate (8-3).

### tert-butyl 3-(6-(2-(1-benzyl-1H-pyrazol-4-yl)thiazole-4-carboxamido)-1-((2-(trimethylsilyl)ethoxy)methyl)-1H-indazol-5-yl)benzylcarbamate (8-4)

Part D: A mixture of tert-butyl 3-(6-(2-bromothiazole-4-carboxamido)-1-((2-(trimethylsilyl)ethoxy)methyl)-1H-indazol-5-yl)benzylcarbamate (8-3) (50 mg, 0.0759 mmol), pyrazole boronate (26 mg, 0.091 mmol), PdCl₂dppf.CH₂Cl₂ (5 mg, 0.00759 mmol), and K₂CO₃ (21 mg, 0.152 mmol) in DMF (1 mL) was stirred at 85° C. for 20 hours under an atmosphere of nitrogen. The reaction was allowed to cool to room temperature, diluted with ethyl acetate, washed with water and brine, dried over sodium sulfate, filtered, concentrated under vacuum, and purified by silica gel chromatography to afford tert-butyl 3-(6-(2-(1-benzyl-1H-pyrazol-4-yl)thiazole-4-carboxamido)-1-((2-(trimethylsilyl)ethoxy)methyl)-1H-indazol-5-yl)benzylcarbamate (8-4) as a colorless oil.

### N-(5-(3-(aminomethyl)phenyl)-1H-indazol-6-yl)-2-(1-benzyl-1H-pyrazol-4-yl)thiazole-4-carboxamide Hydrochloride (8-5)

Part E: A mixture of tert-butyl 3-(6-(2-(1-benzyl-1H-pyrazol-4-yl)thiazole-4-carboxamido)-1-((2-(trimethylsilyl)ethoxy)methyl)-1H-indazol-5-yl)benzylcarbamate (8-4) (17 mg, 0.023 mmol) and HCl (4N in dioxane, 2 mL) in methanol (1 mL) and water (0.5 mL) was stirred at 60° C. for 17 hours. The reaction was allowed to cool to room temperature, concentrated under vacuum, and purified by prep-HPLC to afford N-(5-(3-(aminomethyl)phenyl)-1H-indazol-6-yl)-2-(1-benzyl-1H-pyrazol-4-yl)thiazole-4-carboxamide hydrochloride (8-5) as a white solid. ¹H NMR (400 MHz, CD₃OD) δ 8.73 (s, 1H); 8.15 (s, 1H); 8.13 (s, 1H); 8.07 (s, 1H); 7.73 (s, 1H); 7.70-7.65 (m, 3H); 7.62-7.59 (m, 1H); 7.57-7.51 (m, 1H); 7.44-7.36 (m, 3H), 7.34-7.31 (m, 2H); 5.40 (s, 2H); 4.19 (s, 2H). HPLC t_{R}=6.19 min (UV_{254 nm}). Mass calculated for formula C₂₈H₂₃N₇OS 505.17; observed MH⁺ (ESI MS) 506.9 (m/z).

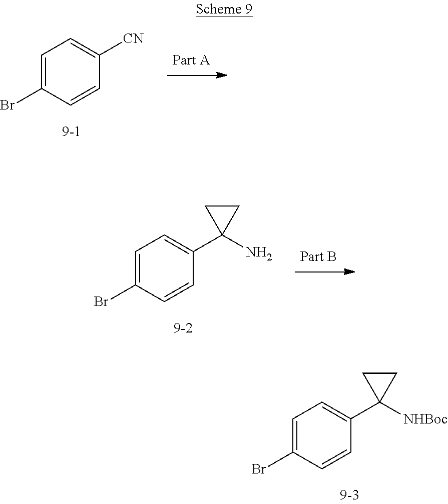 [SMILES: N#Cc1ccc(Br)cc1 ; NC1(c2ccc(Br)cc2)CC1 ; CC(C)(C)OC(=O)NC1(c2ccc(Br)cc2)CC1]

### tert-butyl 1-(4-bromophenyl)cyclopropylcarbamate (9-3)

### 1-(4-bromophenyl)cyclopropanamine (9-2)

Part A: (See reference 4) Ethylmagnesium bromide (2.02 mL, 3M in ether, 6.06 mmol) was added at −78° C. to a solution of 4-bromobenzonitrile (9-1) (500 mg, 2.75 mmol) and Ti(Oi-Pr)₄ (0.89 mL, 3.03 mmol) in Et₂O (14 mL). The reaction was stirred at −78° C. for half an hour. The reaction was warmed up to room temperature and stirred for an hour, then BF₃.OEt₂ (0.69 mL, 5.5 mmol) was added. After the reaction was stirred for an hour, 2N HCl was added. The mixture was stirred at room temperature for 10 minutes, then made basic with 2N NaOH. It was diluted with ethyl acetate, washed with brine, dried over sodium sulfate, filtered, concentrated under vacuum, and purified by silica gel chromatography to afford 1-(4-bromophenyl)cyclopropanamine (9-2).

### tert-butyl 1-(4-bromophenyl)cyclopropylcarbamate (9-3)

Part B: A mixture of 1-(4-bromophenyl)cyclopropanamine (9-2) (300 mg, 1.41 mmol) and Boc₂O (340 mg, 1.55 mmol) was refluxed in toluene (6 mL) for 1 hour. The reaction was allowed to cool to room temperature, concentrated under vacuum, and purified by silica gel chromatography to afford tert-butyl 1-(4-bromophenyl)cyclopropylcarbamate (9-3).

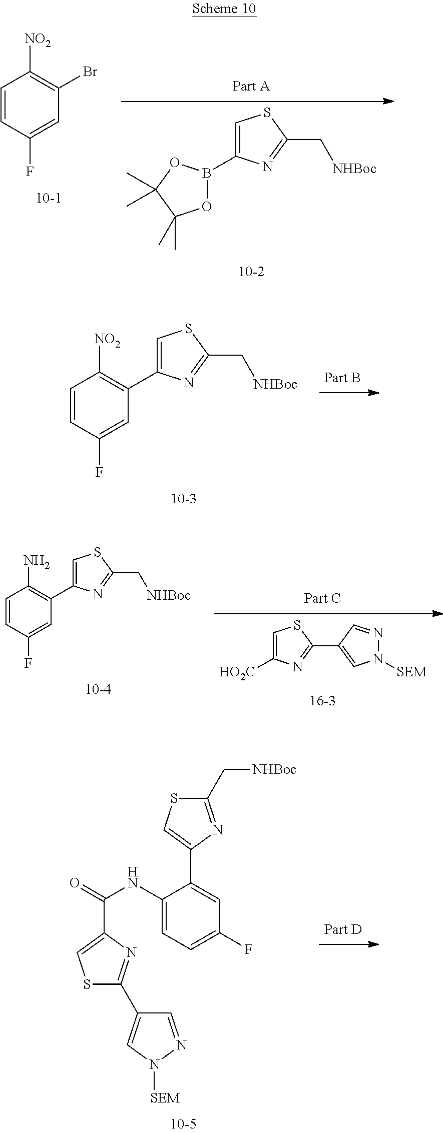 [SMILES: O=[N+]([O-])c1ccc(F)cc1Br ; CC(C)(C)OC(=O)NCc1nc(B2OC(C)(C)C(C)(C)O2)cs1 ; CC(C)(C)OC(=O)NCc1nc(-c2cc(F)ccc2[N+](=O)[O-])cs1 ; CC(C)(C)OC(=O)NCc1nc(-c2cc(F)ccc2N)cs1 ; C[Si](C)(C)CCOCn1cc(-c2nc(C(=O)O)cs2)cn1 ; CC(C)(C)OC(=O)NCc1nc(-c2cc(F)ccc2NC(=O)c2csc(-c3cnn(COCC[Si](C)(C)C)c3)n2)cs1]

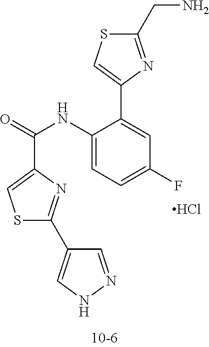 [SMILES: NCc1nc(-c2cc(F)ccc2NC(=O)c2csc(-c3cn[nH]c3)n2)cs1 ; Cl]

### N-(2-(2-(aminomethyl)thiazol-4-yl)-4-fluorophenyl)-2-(1H-pyrazol-4-yl)thiazole-4-carboxamide Hydrochloride (10-6)

### tert-butyl (4-(5-fluoro-2-nitrophenyl)thiazol-2-yl)methylcarbamate (10-3)

Part A: A mixture of 2-bromo-4-fluoro-1-nitrobenzene (10-1) (115 mg, 0.52 mmol), tert-butyl (4-(4,4,5,5-tetramethyl-1,3,2-dioxaborolan-2-yl)thiazol-2-yl)methylcarbamate (10-2) (231 mg, 0.68 mmol), PdCl₂dppf.CH₂Cl₂ (42 mg, 0.052 mmol) and K₂CO₃ (216 mg, 1.56 mmol) in DMF (5 mL) was stirred at 85° C. under a nitrogen atmosphere for 18 hours. The mixture was allowed to cool to room temperature, diluted with ethyl acetate, washed with water and brine, dried over sodium sulfate, filtered, concentrated under vacuum, and purified by silica gel chromatography to afford tert-butyl (4-(5-fluoro-2-nitrophenyl)thiazol-2-yl)methylcarbamate (10-3).

### tert-butyl (4-(2-amino-5-fluorophenyl)thiazol-2-yl)methylcarbamate (10-4)

Part B: A mixture of tert-butyl (4-(5-fluoro-2-nitrophenyl)thiazol-2-yl)methylcarbamate (10-3) (130 mg, 0.37 mmol) and Pd/C (10% Pd, 100 mg, 0.0468 mmol) in ethyl acetate (3.5 mL) was stirred under an atmosphere of hydrogen gas at room temperature for 18 hours. The mixture was then filtered through a pad of Celite and concentrated under vacuum to afford compound tert-butyl (4-(2-amino-5-fluorophenyl)thiazol-2-yl)methylcarbamate (10-4).

### tert-butyl (4-(5-fluoro-2-(2-(1-((2-(trimethylsilyl)ethoxy)methyl)-1H-pyrazol-4-yl)thiazole-4-carboxamido)phenyl)thiazol-2-yl)methylcarbamate (10-5)

Part C: A mixture of tert-butyl (4-(2-amino-5-fluorophenyl)thiazol-2-yl)methylcarbamate (10-4) (103 mg, 0.32 mmol), 2-(1-((2-(trimethylsilyl)ethoxy)methyl)-1H-pyrazol-4-yl)thiazole-4-carboxylic acid (16-3) (104 mg, 0.32 mmol), HATU (182 mg, 0.48 mmol), and DIPEA (124 mg, 0.96 mmol) in DMF (6 mL) was stirred at room temperature for 18 hours. The mixture was diluted with ethyl acetate, washed with saturated aqueous sodium bicarbonate and brine, dried over sodium sulfate, filtered, concentrated under vacuum, and purified by silica gel chromatography to afford compound tert-butyl (4-(5-fluoro-2-(2-(1-((2-(trimethylsilyl)ethoxy)methyl)-1H-pyrazol-4-yl)thiazole-4-carboxamido)phenyl)thiazol-2-yl)methylcarbamate (10-5).

### N-(2-(2-(aminomethyl)thiazol-4-yl)-4-fluorophenyl)-2-(1H-pyrazol-4-yl)thiazole-4-carboxamide Hydrochloride (10-6)

Part D: A mixture of tert-butyl (4-(5-fluoro-2-(2-(1-((2-(trimethylsilyl)ethoxy)methyl)-1H-pyrazol-4-yl)thiazole-4-carboxamido)phenyl)thiazol-2-yl)methylcarbamate (10-5) (178 mg, 0.28 mmol) and HCl (4N in 1,4-dioxane, 3 mL) in 1,4-dioxane (2 mL) and water (1 mL) was stirred at room temperature for 18 hours. The reaction was concentrated under vacuum and purified by prep-HPLC to afford N-(2-(2-(aminomethyl)thiazol-4-yl)-4-fluorophenyl)-2-(1H-pyrazol-4-yl)thiazole-4-carboxamide hydrochloride (10-6) as a white solid. ¹H NMR (400 MHz, DMSO d₆) δ 11.36 (s, 1H); 8.62 (bs, 3H); 8.37-8.33 (m, 3H); 8.27 (bs, 2H); 7.78-7.74 (dd, J₁=9.9 Hz, J₂=3.0 Hz, 1H); 7.36-7.31 (m, 1H); 1.99 (d, J=5.1 Hz, 1H). HPLC t_{R}=4.53 min (UV_{254 nm}). Mass calculated for formula C₁₇H₁₃FN₆OS₂ 400.06; observed MH⁺ (ESI MS) 401.3 (m/z).

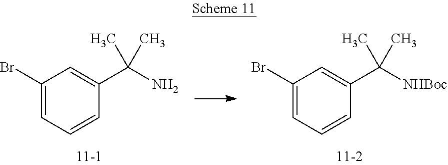 [SMILES: CC(C)(N)c1cccc(Br)c1 ; CC(C)(C)OC(=O)NC(C)(C)c1cccc(Br)c1]

### tert-butyl 2-(3-bromophenyl)propan-2-ylcarbamate (11-2)

A mixture of 2-(3-bromophenyl)propan-2-amine 11-1² (181 mg, 0.845 mmol) and Boc₂O (203 mg, 0.930 mmol) was refluxed in toluene (4 mL) for 1.5 hours. The reaction was allowed to cool to room temperature, concentrated under vacuum, and purified by silica gel chromatography to afford tert-butyl 2-(3-bromophenyl)propan-2-ylcarbamate (11-2) as a colorless oil.

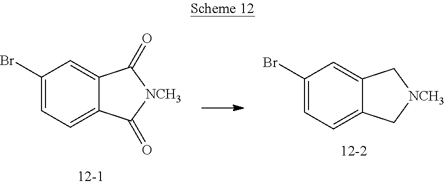 [SMILES: CN1C(=O)c2ccc(Br)cc2C1=O ; CN1Cc2ccc(Br)cc2C1]

### 5-bromo-2-methylisoindoline (12-2)

A mixture of 5-bromo-2-methylisoindoline-1,3-dione (12-1)³ (250 mg, 1.04 mmol) in BH₃.THF (1M, 8 mL) was stirred at reflux for 48 hours. The reaction was allowed to cool to room temperature, quenched with 2N HCl, then made basic with 2N NaOH. The phases were separated and the organics were washed with brine, dried over sodium sulfate, filtered, concentrated under vacuum and purified by silica gel chromatography. The resulting material was dissolved in methanol (4 mL) and concentrated HCl (4 mL) was added and the mixture was stirred at 85° C. for 16 hours. The reaction was allowed to cool to room temperature then diluted with ethyl acetate. The mixture was carefully made basic with 2N NaOH. The phases were separated and the organics were dried over sodium sulfate, filtered, and concentrated under vacuum to afford 5-bromo-2-methylisoindoline (12-2).

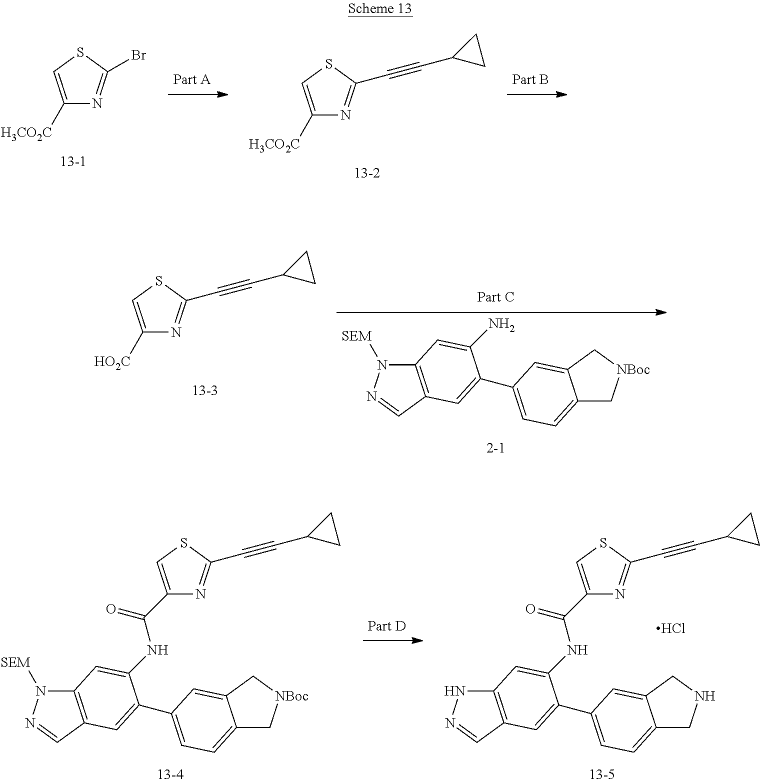

### 2-(cyclopropylethynyl)-N-(5-(isoindolin-5-yl)-1H-indazol-6-yl)thiazole-4-carboxamide Hydrochloride (13-5)

### methyl 2-(cyclopropylethynyl)thiazole-4-carboxylate (13-2)

Part A: A mixture of methyl 2-bromothiazole-4-carboxylate (13-1) (500 mg, 2.25 mmol), cyclopropylacetylene (223 mg, 3.38 mmol)), palladium acetate (51 mg, 0.225 mmol), triphenylphosphine (118 mg, 0.45 mmol), copper iodide (86 mg, 0.45 mmol), and triethylamine (455 mg, 4.5 mmol) in THF (3 mL) was stirred at 40° C. for 17 hours under a nitrogen atmosphere. The mixture was allowed to cool to room temperature, diluted with ethyl acetate, washed with 2N HCl and brine, dried over sodium sulfate, filtered, concentrated under vacuum, and purified by silica gel chromatography to afford methyl 2-(cyclopropylethynyl)thiazole-4-carboxylate (13-2).

### 2-(cyclopropylethynyl)thiazole-4-carboxylic Acid (13-3)

Part B: A mixture of methyl 2-(cyclopropylethynyl)thiazole-4-carboxylate (13-2) (100 mg, 0.483 mmol) and sodium hydroxide (2M, 2.4 mL) in methanol (5 mL) was stirred at room temperature for 1 hour. The mixture was diluted with ethyl acetate, then made acidic with 2N HCl. The phases were separated and the organics were washed with brine, dried over sodium sulfate, filtered, and concentrated under vacuum to afford 2-(cyclopropylethynyl)thiazole-4-carboxylic acid (13-3).

### tert-butyl 5-(6-(2-(cyclopropylethynyl)thiazole-4-carboxamido)-1-((2-(trimethylsilyl)ethoxy)methyl)-1H-indazol-5-yl)isoindoline-2-carboxylate (13-4)

Part C: A mixture of 2-(cyclopropylethynyl)thiazole-4-carboxylic acid (13-3) (54 mg, 0.278 mmol), tert-butyl 5-(6-amino-1-((2-(trimethylsilyl)ethoxy)methyl)-1H-indazol-5-yl)isoindoline-2-carboxylate (2-1) (160 mg, 0.333 mmol), HATU (159 mg, 0.417 mmol), and DIPEA (108 mg, 0.834 mmol) in DMF (2 mL) was stirred at room temperature for 21.5 hours. The mixture was diluted with ethyl acetate, washed with saturated aqueous sodium bicarbonate and brine, dried over sodium sulfate, filtered, concentrated under vacuum, and purified by silica gel chromatography to afford tert-butyl 5-(6-(2-(cyclopropylethynyl)thiazole-4-carboxamido)-1-((2-(trimethylsilyl)ethoxy)methyl)-1H-indazol-5-yl)isoindoline-2-carboxylate (13-4),

### 2-(cyclopropylethynyl)-N-(5-(isoindolin-5-yl)-1H-indazol-6-yl)thiazole-4-carboxamide Hydrochloride (13-5)

Part D: A mixture of tert-butyl 5-(6-(2-(cyclopropylethynyl)thiazole-4-carboxamido)-1-((2-(trimethylsilyl)ethoxy)methyl)-1H-indazol-5-yl)isoindoline-2-carboxylate (13-4) (120 mg, 0.183 mmol) and HCl (4N in dioxane, 3 mL) in methanol (3 mL) was stirred at 60° C. for 1.5 hours. The reaction was allowed to cool to room temperature, concentrated under vacuum, and purified by prep-HPLC to afford 2-(cyclopropylethynyl)-N-(5-(isoindolin-5-yl)-1H-indazol-6-yl)thiazole-4-carboxamide hydrochloride (13-5) as a white solid. ¹H NMR (400 MHz, CD₃OD) δ 8.34 (s, 1H); 8.18 (s, 1H); 8.08 (s, 1H); 7.75 (s, 1H); 7.57-7.54 (m, 3H); 4.70 (d, J=6 Hz, 4H); 1.62-1.59 (m, 1H); 1.04-0.98 (m, 2H); 0.91-0.86 (m, 2H). HPLC t_{R}=4.75 min (UV_{254 nm}). Mass calculated for formula C₂₄H₁₉N₅OS 425.13; observed MH⁺ (ESI MS) 426.1 (m/z).

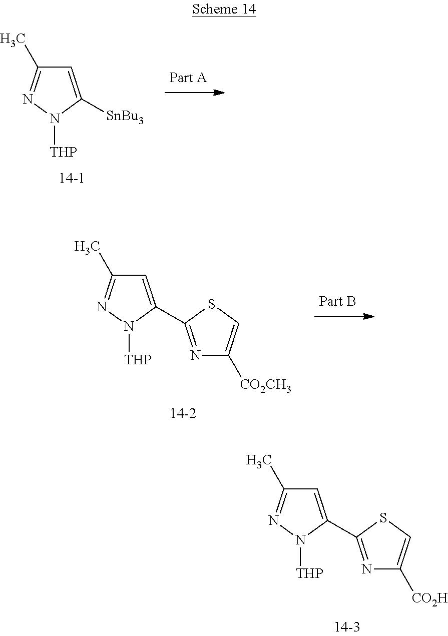

### 2-(3-methyl-1-(tetrahydro-2H-pyran-2-yl)-1H-pyrazol-5-yl)thiazole-4-carboxylic Acid (14-3)

### Methyl 2-(3-methyl-1-(tetrahydro-2H-pyran-2-yl)-1H-pyrazol-5-yl)thiazole-4-carboxylate (14-2)

Part A: A mixture of 3-methyl-1-(tetrahydro-2H-pyran-2-yl)-5-(tributylstannyl)-1H-pyrazole (14-1) (9.20 g, 18.0 mmol), methyl 2-bromothiazole-4-carboxylate (2.00 g, 9.0 mmol) and Pd(PPh₃)₄ (2.08 g, 1.8 mmol) in 1,4-dioxane (10 mL) was stirred at 110° C. for 72 h under a nitrogen atmosphere. The mixture was allowed to cool to room temperature, diluted with ethyl acetate, washed with water and brine, dried over sodium sulfate, filtered, concentrated under vacuum, and purified by silica gel chromatography to afford methyl 2-(3-methyl-1-(tetrahydro-2H-pyran-2-yl)-1H-pyrazol-5-yl)thiazole-4-carboxylate (14-2).

### 2-(3-methyl-1-(tetrahydro-2H-pyran-2-yl)-1H-pyrazol-5-yl)thiazole-4-carboxylic Acid (14-3)

Part B: A solution of sodium hydroxide (2N, 51 mL, 102 mmol) was added to a stirred solution of 2-(3-methyl-1-(tetrahydro-2H-pyran-2-yl)-1H-pyrazol-5-yl)thiazole-4-carboxylate (14-2) (3.14 g, 10.2 mmol) in methanol (51 mL). The mixture was stirred at 25° C. for 18 hours. The reaction mixture was neutralized with HCl (2N, 51 mL, 102 mmol), extract with dichloromethane, washed with brine, dried over sodium sulfate, filtered, and concentrated under vacuum to afford 2-(3-methyl-1-(tetrahydro-2H-pyran-2-yl)-1H-pyrazol-5-yl)thiazole-4-carboxylic acid (14-3).

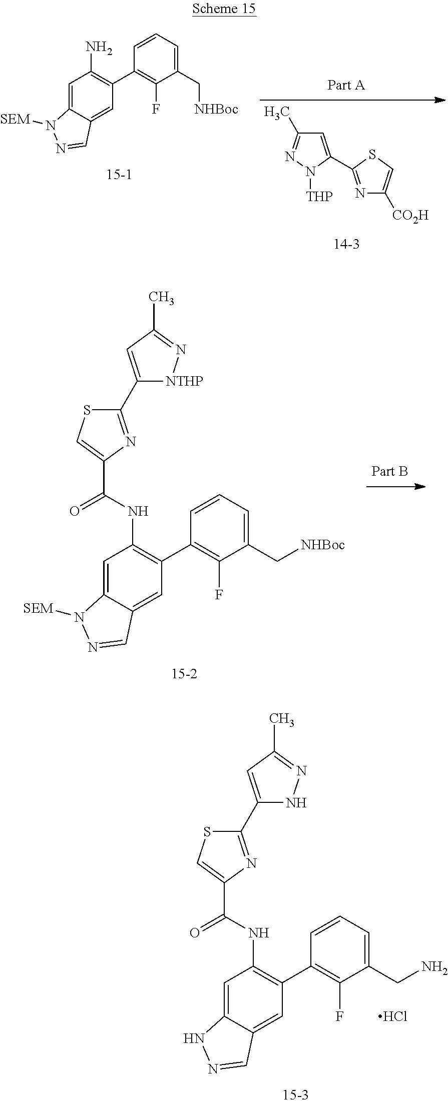

### N-(5-(3-(aminomethyl)-2-fluorophenyl)-1H-indazol-6-yl)-2-(3-methyl-1H-pyrazol-5-yl)thiazole-4-carboxamide Hydrochloride (15-3)

### tert-butyl 2-fluoro-3-(6-(2-(3-methyl-1-(tetrahydro-2H-pyran-2-yl)-1H-pyrazol-5-yl)thiazole-4-carboxamido)-1-((2-(trimethylsilyl)ethoxy)methyl)-1H-indazol-5-yl)benzylcarbamate (15-2)

Part A: A mixture of tert-butyl 3-(6-amino-1-((2-(trimethylsilyl)ethoxy)methyl)-1H-indazol-5-yl)-2-fluorobenzylcarbamate (15-1) (100 mg, 0.205 mmol), 2-(3-methyl-1-(tetrahydro-2H-pyran-2-yl)-1H-pyrazol-5-yl)thiazole-4-carboxylic acid (14-3) (90 mg, 0.308 mmol), HATU (117 mg, 0.308 mmol), and DIPEA (79 mg, 0.615 mmol) in DMF (2 mL) was stirred at room temperature for 18 hours. The mixture was diluted with ethyl acetate, washed with saturated aqueous sodium bicarbonate and brine, dried over sodium sulfate, filtered, concentrated under vacuum, and purified by silica gel chromatography to afford tert-butyl 2-fluoro-3-(6-(2-(3-methyl-1-(tetrahydro-2H-pyran-2-yl)-1H-pyrazol-5-yl)thiazole-4-carboxamido)-1-((2-(trimethylsilyl)ethoxy)methyl)-1H-indazol-5-yl)benzylcarbamate (15-2).

### N-(5-(3-(aminomethyl)-2-fluorophenyl)-1H-indazol-6-yl)-2-(3-methyl-1H-pyrazol-5-yl)thiazole-4-carboxamide Hydrochloride (15-3)

Part B: A mixture of tert-butyl 2-fluoro-3-(6-(2-(3-methyl-1-(tetrahydro-2H-pyran-2-yl)-1H-pyrazol-5-yl)thiazole-4-carboxamido)-1-((2-(trimethylsilyl)ethoxy)methyl)-1H-indazol-5-yl)benzylcarbamate (15-2) (50 mg, 0.069 mmol), and HCl (4N in dioxane, 2 mL, 8.0 mmol) in methanol (1 mL) and water (0.5 mL) was stirred at 60° C. for 16 hours. The reaction was allowed to cool to room temperature, concentrated under vacuum, and purified by prep-HPLC to afford N-(5-(3-(aminomethyl)-2-fluorophenyl)-1H-indazol-6-yl)-2-(3-methyl-1H-pyrazol-5-yl)thiazole-4-carboxamide hydrochloride (15-3). ¹H NMR (400 MHz, DMSO d₆) δ 13.12 (bs, 1H); 9.53 (s, 1H); 8.68 (s, 1H); 8.45 (bs, 3H); 8.35 (s, 1H); 8.11 (s, 1H); 7.87-7.83 (m, 1H);); 7.75 (s, 1H); 7.67-7.63 (m, 1H);); 7.55-7.51 (m, 1H); 6.07 (s, 1H); 4.10 (d, J=5 Hz, 2H); 2.38 (s, 3H). HPLC t_{R}=6.14 min (UV_{254 nm}). Mass calculated for formula C₂₂H₁₈FN₇OS 447.13; observed MH⁻ 446.3 (ESI MS) (m/z).

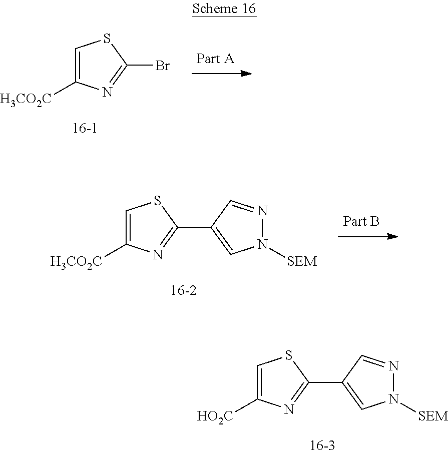 [SMILES: COC(=O)c1csc(Br)n1 ; COC(=O)c1csc(-c2cnn(COCC[Si](C)(C)C)c2)n1 ; C[Si](C)(C)CCOCn1cc(-c2nc(C(=O)O)cs2)cn1]

### 2-(1-((2-(trimethylsilyl)ethoxy)methyl)-1H-pyrazol-4-yl)thiazole-4-carboxylic Acid (16-3) methyl 2-(1-((2-(trimethylsilyl)ethoxy)methyl)-1H-pyrazol-4-yl)thiazole-4-carboxylate (16-2)

Part A: A mixture of methyl 2-bromothiazole-4-carboxylate (16-1) (2.00 g, 9.01 mmol), 4-(4,4,5,5-tetramethyl-1,3,2-dioxaborolan-2-yl)-1-((2-(trimethylsilyl)ethoxy)methyl)-1H-pyrazole (4.38 g, 13.5 mmol), Pd(PPh₃)₄ (1.04 g, 0.901 mmol), and K₂CO₃ (2.49 g, 18.0 mmol) in dioxane (20 mL) was stirred in an 110° C. oil bath under a nitrogen atmosphere for 16 hours. The reaction was allowed to cool to room temperature, diluted with ethyl acetate, washed with water and brine, dried over sodium sulfate, filtered, concentrated under vacuum and purified by silica gel chromatography to afford methyl 2-(1-((2-(trimethylsilyl)ethoxy)methyl)-1H-pyrazol-4-yl)thiazole-4-carboxylate (16-2).

Part B: A solution of aqueous lithium hydroxide (2M, 30.1 mmol) was added to a stirring solution of methyl 2-(1-((2-(trimethylsilyl)ethoxy)methyl)-1H-pyrazol-4-yl)thiazole-4-carboxylate (16-2) (1.75 g, 5.16 mmol) in THF (15 mL) at room temperature and the mixture was stirred for 2.5 hours. The reaction mixture was diluted with ethyl acetate and the pH adjusted to 5 by addition of 2N HCl. The phases were separated and the organics were washed with brine, dried over sodium sulfate, filtered, and concentrated under vacuum to afford 2-(1-((2-(trimethylsilyl)ethoxy)methyl)-1H-pyrazol-4-yl)thiazole-4-carboxylic acid (16-3) as a pale yellow solid.

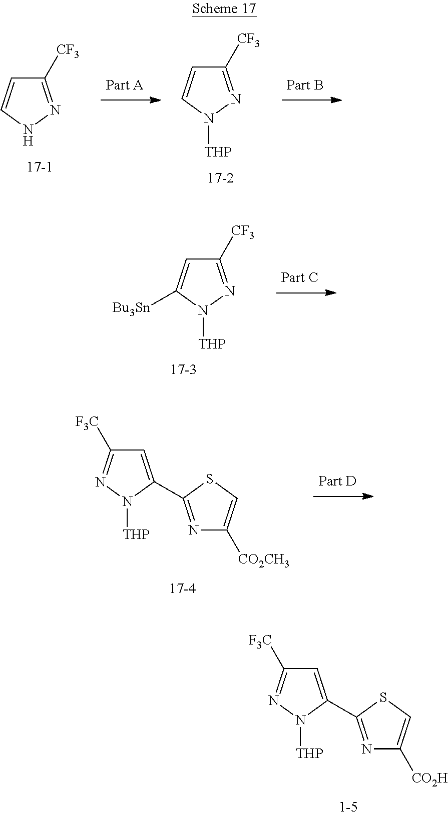

### 2-(1-(tetrahydro-2H-pyran-2-yl)-3-(trifluoromethyl)-1H-pyrazol-5-yl)thiazole-4-carboxylic Acid (1-5)

### 1-(tetrahydro-2H-pyran-2-yl)-3-(trifluoromethyl)-1H-pyrazole (17-2)

Part A: A mixture of 3-(trifluoromethyl)pyrazole (17-1) (2.00 g, 14.7 mmol), 3,4-dihydro-2H-pyran (1.85 g, 22.0 mmol), and TFA (71 mg, 0.735 mmol) in toluene (15 mL) was stirred at room temperature until the reaction was deemed complete as judged by TLC analysis. The mixture was diluted with ethyl acetate, washed with saturated aqueous sodium bicarbonate and brine, dried over sodium sulfate, filtered, concentrated under vacuum, and purified by silica gel chromatography to afford compound 1-(tetrahydro-2H-pyran-2-yl)-3-(trifluoromethyl)-1H-pyrazole (17-2).

Part B: To a solution of compound 1-(tetrahydro-2H-pyran-2-yl)-3-(trifluoromethyl)-1H-pyrazole (17-2) (2.41 g, 11.0 mmol) in THF (40 mL) was added n-BuLi (2N in hexane, 5.5 mL) at −78° C. under a nitrogen atmosphere. The mixture was allowed to warm to −45° C., then stirred for 1 hour. The reaction mixture was cooled to −78° C. and tributyltinchloride (3.92 g, 12.1 mmol) was added. The reaction mixture was allowed to warm to room temperature. The mixture was diluted with ethyl acetate, washed with water and brine, dried over sodium sulfate, filtered, concentrated under vacuum, and purified by silica gel chromatography to afford 1-(tetrahydro-2H-pyran-2-yl)-5-(tributylstannyl)-3-(trifluoromethyl)-1H-pyrazole (17-3).

Part C: A mixture of 1-(tetrahydro-2H-pyran-2-yl)-5-(tributylstannyl)-3-(trifluoromethyl)-1H-pyrazole (17-3) (1.88 g, 3.70 mmol), methyl 2-bromothiazole-4-carboxylate (0.41 g, 1.85 mmol) and Pd(PPh₃)₄ (0.85 g, 0.74 mmol) in 1,4-dioxane (5 mL mL) was stirred at 110° C. for 72 h under a nitrogen atmosphere. The mixture was allowed to cool to room temperature, diluted with ethyl acetate, washed with water and brine, dried over sodium sulfate, filtered, concentrated under vacuum, and purified by silica gel chromatography to afford methyl 2-(1-(tetrahydro-2H-pyran-2-yl)-3-(trifluoromethyl)-1H-pyrazol-5-yl)thiazole-4-carboxylate (17-4).

Part D: A solution of sodium hydroxide (2N, 4 mL, 8.0 mmol) was added to a stirred solution of methyl 2-(1-(tetrahydro-2H-pyran-2-yl)-3-(trifluoromethyl)-1H-pyrazol-5-yl)thiazole-4-carboxylate (17-4) (285 mg, 0.79 mmol) in methanol (4 mL). The mixture was stirred at 25° C. for 18 hours. The reaction mixture was neutralized with HCl (2N, 4 mL, 8.0 mmol), extracted with dichloromethane, washed with brine, dried over sodium sulfate, filtered, and concentrated under vacuum to give 2-(1-(tetrahydro-2H-pyran-2-yl)-3-(trifluoromethyl)-1H-pyrazol-5-yl)thiazole-4-carboxylic acid (1-5).

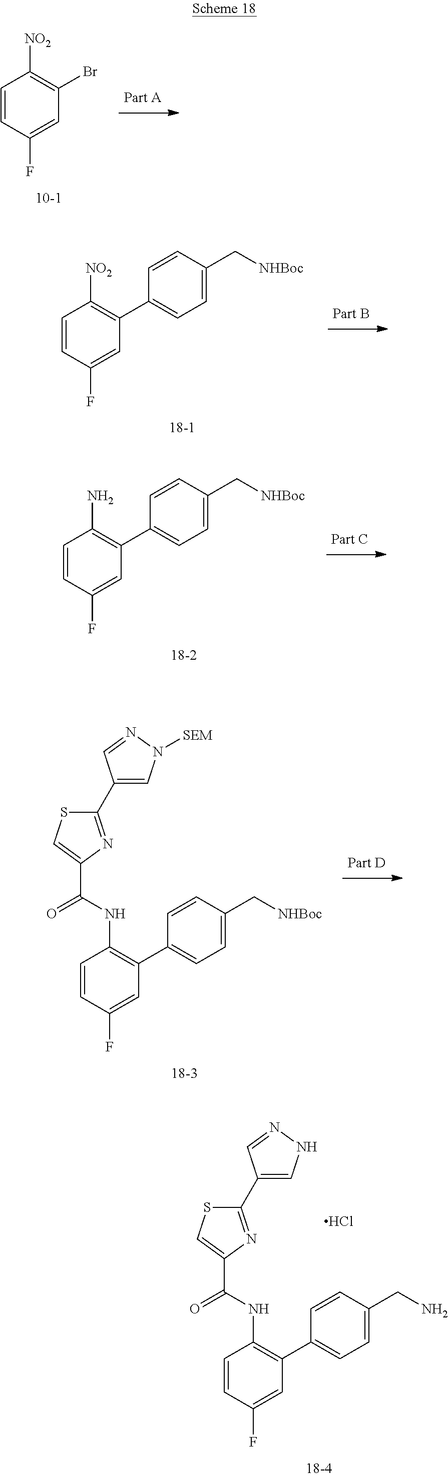 [SMILES: O=[N+]([O-])c1ccc(F)cc1Br ; CC(C)(C)OC(=O)NCc1ccc(-c2cc(F)ccc2[N+](=O)[O-])cc1 ; CC(C)(C)OC(=O)NCc1ccc(-c2cc(F)ccc2N)cc1 ; CC(C)(C)OC(=O)NCc1ccc(-c2cc(F)ccc2NC(=O)c2csc(-c3cnn(COCC[Si](C)(C)C)c3)n2)cc1 ; NCc1ccc(-c2cc(F)ccc2NC(=O)c2csc(-c3cn[nH]c3)n2)cc1 ; Cl]

### N-(4′-(aminomethyl)-5-fluorobiphenyl-2-yl)-2-(1H-pyrazol-4-yl)thiazole-4-carboxamide Hydrochloride (18-4)

### tert-butyl (5′-fluoro-2′-nitrobiphenyl-4-yl)methylcarbamate (18-1)

Part A: A mixture of 2-bromo-4-fluoro-1-nitrobenzene (10-1) (300 mg, 1.36 mmol), tert-butyl 4-(4,4,5,5-tetramethyl-1,3,2-dioxaborolan-2-yl)benzylcarbamate (411 mg, 1.64 mmol), Pd(PPh₃)₄ (157 mg, 0.136 mmol), and K₂CO₃ (375 mg, 2.72 mmol) in DMF (5 mL) was stirred at 85° C. under a nitrogen atmosphere for 16 hours. The reaction was allowed to cool to room temperature, diluted with ethyl acetate, washed with water and brine, dried over sodium sulfate, filtered, concentrated under vacuum and purified by silica gel chromatography to give tert-butyl (5′-fluoro-2′-nitrobiphenyl-4-yl)methylcarbamate (18-1).

### tert-butyl (2′-amino-5′-fluorobiphenyl-4-yl)methylcarbamate (18-2)

Part B: A mixture of tert-butyl (5′-fluoro-2′-nitrobiphenyl-4-yl)methylcarbamate (18-1) (211 mg, 0.609 mmol) and Pd/C (10% Pd, 259 mg, 0.122 mmol) in ethyl acetate (4 mL) was stirred under a hydrogen atmosphere for 17 hours. The mixture was filtered through a pad of Celite and concentrated under vacuum to give tert-butyl (2′-amino-5′-fluorobiphenyl-4-yl)methylcarbamate (18-2).

### tert-butyl (5′-fluoro-2′-(2-(1-((2-(trimethylsilyl)ethoxy)methyl)-1H-pyrazol-4-yl)thiazole-4-carboxamido)biphenyl-4-yl)methylcarbamate (18-3)

Part C: A mixture of tert-butyl (2′-amino-5′-fluorobiphenyl-4-yl)methylcarbamate (18-2) (178 mg, 0.563 mmol), 2-(1-((2-(trimethylsilyl)ethoxy)methyl)-1H-pyrazol-4-yl)thiazole-4-carboxylic acid (16-3) (153 mg, 0.469 mmol), HATU (267 mg, 0.704 mmol), and DIPEA (182 mg, 1.41 mmol) in DMF (7 mL) was stirred at room temperature for five hours. The mixture was diluted with ethyl acetate, washed with saturated aqueous sodium bicarbonate and brine, dried over sodium sulfate, filtered, concentrated under vacuum, and purified by silica gel chromatography to give tert-butyl (5′-fluoro-2′-(2-(1-((2-(trimethylsilyl)ethoxy)methyl)-1H-pyrazol-4-yl)thiazole-4-carboxamido)biphenyl-4-yl)methylcarbamate (18-3).

### N-(4′-(aminomethyl)-5-fluorobiphenyl-2-yl)-2-(1H-pyrazol-4-yl)thiazole-4-carboxamide Hydrochloride (18-4)

Part D: A mixture of tert-butyl (5′-fluoro-2′-(2-(1-((2-(trimethylsilyl)ethoxy)methyl)-1H-pyrazol-4-yl)thiazole-4-carboxamido)biphenyl-4-yl)methylcarbamate (18-3) (224 mg, 0.359 mmol) and HCl (4N in dioxane, 4 mL) in dioxane (4 mL) was stirred at room temperature for 2 hours. The reaction mixture was concentrated under vacuum and purified on prep-HPLC to give N-(4′-(aminomethyl)-5-fluorobiphenyl-2-yl)-2-(1H-pyrazol-4-yl)thiazole-4-carboxamide hydrochloride (18-4) as a white solid. ¹H NMR (400 MHz, DMSO d₆) δ 9.74 (s, 1H); 8.42 (bs, 3H); 8.23 (s, 1H); 8.18-8.16 (m, 1H); 8.03 (bs, 1H); 7.66-7.59 (m, 4H); 7.34-7.29 (m, 1H); 7.23-7.19 (m, 1H); 4.14-4.10 (m, 2H). HPLC t_{R}=4.67 min (UV_{254 nm}). Mass calculated for formula C₂₀H₁₆FN₅OS 393.11; observed MH⁺ 394.3 (ESI MS) (m/z).

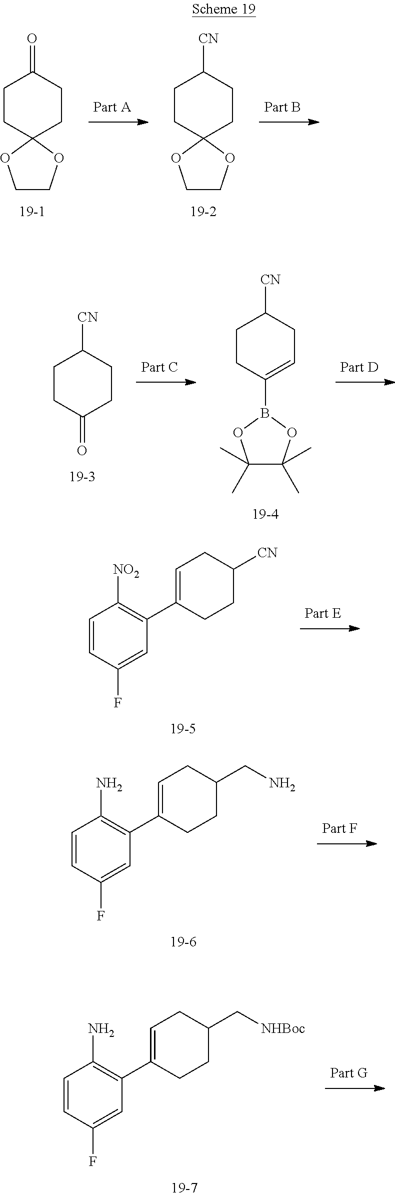

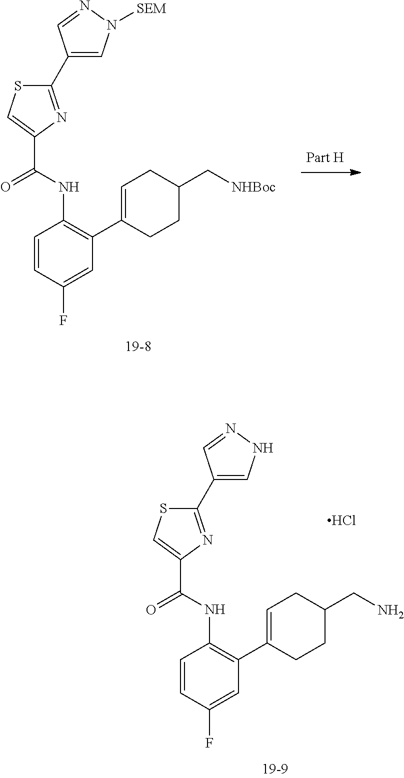 [SMILES: CC(C)(C)OC(=O)NCC1CC=C(c2cc(F)ccc2NC(=O)c2csc(-c3cnn(COCC[Si](C)(C)C)c3)n2)CC1 ; NCC1CC=C(c2cc(F)ccc2NC(=O)c2csc(-c3cn[nH]c3)n2)CC1 ; Cl]

### N-(2-(4-(aminomethyl)cyclohex-1-enyl)-4-fluorophenyl)-2-(1H-pyrazol-4-yl)thiazole-4-carboxamide Hydrochloride (19-9)

### 1,4-dioxaspiro[4,5]decane-8-carbonitrile (19-2)

Part A: A solution of potassium tert-butoxide (5.60 g, 50.0 mmol) in a 1:1 mixture of tert-butanol and 1,2-dimethoxyethane (60 mL) was added to a solution of 1,4-cyclohexanedione monoethylene ketal (19-1) (3.80 g, 24.4 mmol) and tosylmethyl isocyanide (5.03 g, 25.6 mmol) in dimethoxyethane (50 mL) at 0° C. The reaction mixture was stirred at 0° C. for 1 h, then allowed to warm up to room temperature and stir for 1 h. The reaction mixture was diluted with water and extracted with ether. The combined ether solution was dried over sodium sulfate, filtered, and concentrated under vacuum to give 1,4-dioxaspiro[4,5]decane-8-carbonitrile (19-2). ¹H NMR (300 MHz, CDCl₃) δ 3.94-3.95 (m, 4H), 2.61-2.70 (m, 1H), 1.92-1.98 (m, 4H), 1.82-1.88 (m, 2H), 1.56-1.66 (m, 2H).

### 4-oxocyclohexanecarbonitrile (19-3)

Part B: To a solution of 1,4-dioxaspiro[4,5]decane-8-carbonitrile (19-2) (6.30 g, 37.7 mmol) in CH₃CN (100 mL) and H₂O (50 mL) was added a solution of ammonium cerium (IV) nitrate (2.06 g, 3.77 mmol) in H₂O (25 mL). The reaction was heated at 70° C. for 1 h. The mixture was cooled and diluted with H₂O, extracted with ether and dried over sodium sulfate, filtered, concentrated under vacuum and purified by silica gel chromatography to give 4-oxocyclohexanecarbonitrile (19-3). ¹H NMR (300 MHz, CDCl₃) δ 3.00-3.06 (m, 1H), 2.58-2.68 (m, 2H), 2.38-2.47 (m, 2H), 2.10-2.28 (m, 4H).

### 4-(4,4,5,5-tetramethyl-1,3,2-dioxaborolan-2-yl)cyclohex-3-enecarbonitrile (19-4)

Part C: To a solution of 4-oxocyclohexanecarbonitrile (19-3) (1.00 g, 8.12 mmol) in THF (10 mL) at −78° C. was added LiHMDS (10.5 mL, 10.5 mmol, 1M in THF). The reaction was stirred at −78° C. for 0.5 h, then a solution of PhNTf₂ (3.50 g, 9.74 mmol) in THF (2 mL) was added, the reaction was slowly warmed up to room temperature and stirred at room temperature overnight. The reaction was quentioned with H₂O, extracted with EtOAc and dried over sodium sulfate. The organic solution was filtered and concentrated under vacuum. The resulting residue was dissolved in 1,4-dioxanes (18 mL), and bis(pinacolato)diboron (3.00 g, 12.2 mmol), KOAc (1.59 g, 16.2 mmol), and PdCl₂(dppf)₂ (0.22 g, 0.30 mmol) were added. The reaction mixture was degassed and back-filled with N₂. The reaction mixture was stirred at 80° C. for 5 h. The mixture was cooled and filtered through a pad of Celite. The filtrate was concentrated under vacuum and purified by silica gel chromatography to give 4-(4,4,5,5-tetramethyl-1,3,2-dioxaborolan-2-yl)cyclohex-3-enecarbonitrile (19-4).

### 4-(5-fluoro-2-nitrophenyl)cyclohex-3-enecarbonitrile (19-5)

Part D: A mixture of 4-(4,4,5,5-tetramethyl-1,3,2-dioxaborolan-2-yl)cyclohex-3-enecarbonitrile (19-4) (1.3 g, 5.45 mmol), 2-bromo-4-fluoro-1-nitrobenzene (10-1) (1.8 g, 8.18 mmol), Pd(PPh₃)₄ (0.63 g, 0.55 mmol), and K₂CO₃ (2.3 g, 16.4 mmol) in DMF (10 mL) was stirred under a nitrogen atmosphere at 85° C. for 16 hours. The mixture was allowed to cool to room temperature, diluted with ethyl acetate, washed with water and brine, dried over sodium sulfate, filtered, concentrated under vacuum, and purified by silica gel chromatography to afford 4-(5-fluoro-2-nitrophenyl)cyclohex-3-enecarbonitrile (19-5).

### 2-(4-(aminomethyl)cyclohex-1-enyl)-4-fluoroaniline (19-6)

Part E: To a solution of 4-(5-fluoro-2-nitrophenyl)cyclohex-3-enecarbonitrile (19-5) (0.42 g, 1.71 mmol) in ethanol (30 mL) was added Raney-Ni (˜1.5 g). The reaction was stirred in Parr-shaker at room temperature under H₂ (45 psi) overnight. The mixture was diluted with methanol and filtered through a pad of Celite. The filtrate was concentrated under vacuum and purified by silica gel chromatography to give 2-(4-(aminomethyl)cyclohex-1-enyl)-4-fluoroaniline (19-6). ¹H NMR (300 MHz, CDCl₃) δ 6.68-6.77 (m, 2H), 6.59-6.63 (m, 1H), 5.77-5.78 (m, 1H), 3.60 (bs, 2H), 2.69 (d, J=6.3 Hz, 2H), 2.29-2.35 (m, 3H), 1.80-1.94 (m, 2H), 1.67-1.69 (m, 1H), 1.33-1.46 (m, 1H).

### tert-butyl (4-(2-amino-5-fluorophenyl)cyclohex-3-enyl)methylcarbamate (19-7)

Part F: A mixture of 2-(4-(aminomethyl)cyclohex-1-enyl)-4-fluoroaniline (19-6) (115 mg, 0.52 mmol), Boc₂O (115 mg, 0.52 mmol), DMAP (7 mg, 0.05 mmol) and TEA (0.22 mL, 1.56 mmol) in dichloromethane (5 mL) was stirred at room temperature for 16 hours. The reaction was diluted with ethyl acetate, washed with saturated aqueous ammonium chloride and brine, dried over sodium sulfate, filtered, concentrated under vacuum and purified by silica gel chromatography to afford of tert-butyl (4-(2-amino-5-fluorophenyl)cyclohex-3-enyl)methylcarbamate (19-7).

### tert-butyl (4-(5-fluoro-2-(2-(1-((2-(trimethylsilyl)ethoxy)methyl)-1H-pyrazol-4-yl)thiazole-4-carboxamido)phenyl)cyclohex-3-enyl)methylcarbamate (19-8)

Part G: A mixture of tert-butyl (4-(2-amino-5-fluorophenyl)cyclohex-3-enyl)methylcarbamate (19-7) (50 mg, 0.16 mmol), 2-(1-((2-(trimethylsilyl)ethoxy)methyl)-1H-pyrazol-4-yl)thiazole-4-carboxylic acid (16-3) (56 mg, 0.17 mmol), HATU (89 mg, 0.23 mmol), and DIPEA (61 mg, 0.47 mmol) in DMF (4 mL) was stirred at room temperature for 16 hours. The reaction was diluted with ethyl acetate, washed with water and brine, dried over sodium sulfate, filtered, concentrated under vacuum, and purified by silica gel chromatography to give tert-butyl (4-(5-fluoro-2-(2-(1-((2-(trimethylsilyl)ethoxy)methyl)-1H-pyrazol-4-yl)thiazole-4-carboxamido)phenyl)cyclohex-3-enyl)methylcarbamate (19-8).

### N-(2-(4-(aminomethyl)cyclohex-1-enyl)-4-fluorophenyl)-2-(1H-pyrazol-4-yl)thiazole-4-carboxamide Hydrochloride (19-9)

Part H: A mixture of tert-butyl (4-(5-fluoro-2-(2-(1-((2-(trimethylsilyl)ethoxy)methyl)-1H-pyrazol-4-yl)thiazole-4-carboxamido)phenyl)cyclohex-3-enyl)methylcarbamate (19-8) (90 mg, 0.148 mmol) and HCl (4N in dioxane, 4 mL) in 1,4-dioxane (5 mL) was stirred at room temperature for 2 hours. The reaction was concentrated under vacuum and purified by prep-HPLC to give N-(2-(4-(aminomethyl)cyclohex-1-enyl)-4-fluorophenyl)-2-(1H-pyrazol-4-yl)thiazole-4-carboxamide hydrochloride (19-9). ¹H NMR (300 MHz, DMSO d₆) δ 9.72 (s, 1H); 8.32 (s, 1H); 8.22-8.24 (m, 3H); 7.96 (bs, 3H); 7.16-7.11 (m, 2H); 5.86 (s, 1H); 2.86-2.81 (m, 2H); 2.40-2.25 (m, 3H); 2.00-1.96 (m, 3H); 1.55-1.40 (m, 1H). HPLC t_{R}=4.85 min (UV_{254 nm}). Mass calculated for formula C₂₀H₂₀FN₅OS 397.14; observed MH⁺ (ESI MS) 398.20 (m/z).

### Biological Assay

The assay used to test the compounds' abilities to inhibit phosphorylation of a substrate by PDK1 uses the IMAP® technology system available from Molecular Devices (Silicon Valley, CA, United States). The technology enables the detection of the phosphorylation of protein substrates by PDK1 and does not require the addition of antibodies to detect substrate phosphorylation. The technology is based on the high-affinity interaction of trivalent metal containing nanoparticles (beads) with phospho-groups on the substrate of interest. The readout for the assay was fluorescence polarization (FP) which increased once the fluorescently labeled substrate was phosphorylated and was bound to the beads as opposed to the unphosphorylated substrate which did not bind the beads and had relatively lower polarization.

In a microwell assay format, the fluorescently-labeled peptide substrate from glycogen synthase-1 (5FAM-PLSRTLSVSSLPGL-NH2 (SEQ ID NO:1) Molecular Devices part no RP7045). was phosphorylated in a kinase reaction. Addition of the IMAP® Binding System (available from Molecular Devices) stopped the kinase reaction and specifically bound the phosphorylated substrates. Phosphorylation and subsequent binding of the substrate to the beads was detected by FP.

The PDK1 IMAP assay utilized recombinant human PDK1 produced in Sf9 insect cells and containing amino acids 51-556 of the human PDK1 enzyme. The assay measured the change in fluorescence polarization caused by phosphorylation of a peptide substrate by PDK1. Addition of small molecule PDK1 inhibitors results in the reduction of peptide phosphorylation changing the fluorescence polarization which is measured using a fluorescence plate reader. The assay was performed in a 384-well plate with 10 nM PDK1 enzyme, 100 nM peptide substrate 1 (SEQ ID NO:1), 100 nM activated peptide PIFtide and 2.5 uM ATP for 1.5 hours. PIFtide is added separately to the IMAP reaction at 100 nM. The peptide sequence of PIFTtide is RREPRILSEEEQEMFRDFDYIADWC (SEQ ID NO:2). PIFtide is a peptide sequence that interacts with PDK-1 and is derived from PRK2 kinase, a PDK-1 substrate. This sequence is present in the hydrophobic motif present in PDK-1 substrates and binds to the kinase domain of PDK-1. It is thought to act as a docking site for PDK-1 on the substrate and in vitro has been shown enhance PDK-1 phosphorylation of substrates by approximately 4-fold. See Biondi et al., EMBO 19, 979-988 (2000).

The detection beads were then added and allowed to incubate for 1 hour at room temperature and the fluorescence was then read. Staurosporine, a broad spectrum kinase inhibitor, was used as a positive control for the assay resulting in typical IC₅₀s of 3 nM. Test compounds in 100% DMSO at a range of concentrations were added at 0.5 μl 15 minutes prior to ATP addition. The fluorescence polarization units (mP) generated with 1 uM stauroporine is considered to be background mP and the mP units generated with DMSO is considered to be total mP for each assay. The IC₅₀ value is calculated based on fitting the mP units to the total and background mP and the concentration required to inhibit the mP units by 50% is reported to be the IC₅₀.

The compounds of the instant invention that are described in the Schemes 1-19, and the associated Tables, have IC₅₀ values less than 5 μM. Table 6 below lists representative compounds of the invention with activity data.

TABLE 6

| Compound | IC₅₀ | |
|---|---|---|
| 1-11 | 33 | nM |
| 1-18 | 1855 | nM |
| 3-5 | 170 | nM |
| 4-6 | 108 | nM |
| 5-7 | 85 | nM |

What is claimed is:

1. A compound of the Formula A, 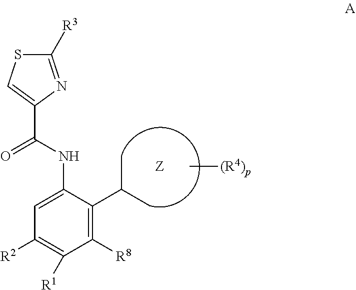
wherein,
a is 0 or 1, b is 0 or 1, m is 0, 1 or 2, and p is 0, 1, 2, 3 or 4;
ring Z is attached to phenyl via a carbon-carbon bond and is selected from cycloalkyl, cycloalkenyl, aryl, heteroaryl and heterocyclyl;
R¹ and R² are independently selected from CF₃, CN, halo, OH, C₁₋₆ alkyl and C₃₋₈ cycloalkyl, or R¹ and R² can be taken together to form a heterocyclyl which is optionally substituted with one to four substituents selected from CF₃, halo, N(R^{b})₂, OH, (O)C₁₋₃ alkyl, C₁₋₃ alkyl and C₃₋₆ cycloalkyl;
R³ is selected from hydrogen, CF₃, CN, halo, CO₂H, C₁₋₆ alkyl, C₂₋₆ alkenyl, C₂₋₆ alkynyl, C₃₋₈ cycloalkyl, C₃₋₈ cycloalkenyl, aryl, heteroaryl, heterocyclyl, N(R^{a})₂, C(═O)N(R^{a})₂, S(O)ₘ C₁₋₆ alkyl, S(O)ₘ C₃₋₈ cycloalkyl, S(O)ₘ C₃₋₈ cycloalkenyl, S(O)ₘ aryl, S(O)ₘ heteroaryl, S(O)ₘN(R^{a})₂, wherein said alkyl, alkenyl, alkynyl, cycloalkyl, cycloalkenyl, aryl, heteroaryl and heterocyclyl are optionally substituted with one to four substituents selected from CF₃, halo, OH, (O)C₁₋₆ alkyl, C₁₋₆ alkyl, C₃₋₆ cycloalkyl, aryl, heteroaryl, heterocyclyl, N(R^{a})₂, C(═O)N(R^{a})₂, S(O)ₘ C₁₋₆ alkyl, S(O)ₘ C₃₋₈ cycloalkyl, S(O)ₘ aryl, S(O)ₘ heteroaryl, S(O)ₘN(R^{a})₂, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, aryl and heterocyclyl;
R⁴ is independently selected from CF₃, CN, halo, CO₂H, OH, C₁₋₆ alkyl, S(O)ₘ C₁₋₆ alkyl, N(R^{a})₂, C(═O)N(R^{a})₂, S(O)ₘN(R^{a})₂, C₃₋₈ cycloalkyl, C₃₋₈ cycloalkenyl, aryl, heteroaryl and heterocyclyl, wherein said alkyl, cycloalkyl, cycloalkenyl, aryl, heteroaryl and heterocyclyl are optionally substituted with one to four substituents selected from CF₃, halo, N(R^{a})₂, C(═O)N(R^{a})₂, OH, (O)C₁₋₆ alkyl, C₁₋₆ alkyl, C₃₋₆ cycloalkyl, aryl, heteroaryl, and heterocyclyl, wherein said alkyl, cycloalkyl, aryl, heteroaryl and heterocyclyl are optionally substituted with one to two substituents selected from CF₃, halo, N(R^{a})₂, C(═O)N(R^{a})₂, OH, (O)C₁₋₃ alkyl, C₁₋₃ alkyl and C₃₋₆ cycloalkyl;
R⁸ is hydrogen or halo;
R^{a} is independently selected from hydrogen, C₁₋₃ alkyl and C₃₋₆ cycloalkyl, wherein said alkyl and cycloalkyl are optionally substituted with CF₃, halo, N(R^{b})₂, OH, (O)C₁₋₃ alkyl, C₁₋₃ alkyl, C₃₋₆ cycloalkyl and phenyl; and
R^{b} is independently selected from hydrogen and C₁₋₃ alkyl;
or a pharmaceutically acceptable salt, tautomer or a stereoisomer thereof.

2. The compound according to claim 1 of the Formula A, wherein,
ring Z is attached to phenyl via a carbon-carbon bond and is selected from cyclohexenyl, pyridyl, isoindolinyl, isoquinolinyl, indenyl and phenyl;
R¹ and R² are independently selected from CF₃, CN, halo, OH, C₁₋₆ alkyl and C₃₋₈ cycloalkyl, or R¹ and R² can be taken together to form an imidazole, pyrrole, pyrazole, thiophene or furan;
R³ is pyrazolyl and C₂₋₆ alkenyl which are optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl; and
all other substituents are as defined in claim 1;
or a pharmaceutically acceptable salt, tautomer or a stereoisomer thereof.

3. The compound according to claim 2 of the Formula A, wherein,
R³ is pyrazolyl which is optionally substituted with one to two substituents selected from CF₃, halo, N(R^{a})₂, C(═O)N(R^{a})₂, OH, (O)C₁₋₃ alkyl, C₁₋₃ alkyl, C₃₋₆ cycloalkyl, phenyl and heteroaryl wherein said alkyl is optionally substituted with phenyl or heterocyclyl; and
all other substituents are as defined in claim 2;
or a pharmaceutically acceptable salt, tautomer or a stereoisomer thereof.

4. The compound according to claim 3 of the Formula A, wherein,
R³ is pyrazolyl which is optionally substituted with CF₃, C₁₋₃ alkyl, phenyl and heteroaryl, wherein said alkyl is optionally substituted with phenyl or heterocyclyl; and
all other substituents are as defined in claim 3;
or a pharmaceutically acceptable salt, tautomer or a stereoisomer thereof.

5. The compound according to claim 4 of the Formula A, wherein,
R³ is pyrazolyl which is optionally substituted with CF₃; and
all other substituents are as defined in claim 4;
or a pharmaceutically acceptable salt, tautomer or a stereoisomer thereof.

6. A compound which is selected from:
N-(5-(isoindolin-5-yl)-1H-indazol-6-yl)-2-(3-(trifluoromethyl)-1H-pyrazol-5-yl)thiazole-4-carboxamide (1-7);
N-(5-(4-(aminomethyl)phenyl)-1H-indazol-6-yl)-2-(3-(trifluoromethyl)-1H-pyrazol-5-yl)thiazole-4-carboxamide (1-8);
N-(5-(3-(aminomethyl)phenyl)-1H-indazol-6-yl)-2-(3-(trifluoromethyl)-1H-pyrazol-5-yl)thiazole-4-carboxamide (1-9);
(R)—N-(5-(3-(1-aminoethyl)phenyl)-1H-indazol-6-yl)-2-(3-(trifluoromethyl)-1H-pyrazol-5-yl)thiazole-4-carboxamide (1-10);
N-(5-(3-(pyrrolidin-1-ylmethyl)phenyl)-1H-indazol-6-yl)-2-(3-(trifluoromethyl)-1H-pyrazol-5-yl)thiazole-4-carboxamide (1-11);
N-(5-(3-(aminomethyl)-4-methylphenyl)-1H-indazol-6-yl)-2-(3-(trifluoromethyl)-1H-pyrazol-5-yl)thiazole-4-carboxamide (1-12);
(S)—N-(5-(3-(1-aminoethyl)phenyl)-1H-indazol-6-yl)-2-(3-(trifluoromethyl)-1H-pyrazol-5-yl)thiazole-4-carboxamide (1-13);
N-(5-(5-(aminomethyl)-2-fluorophenyl)-1H-indazol-6-yl)-2-(3-(trifluoromethyl)-1H-pyrazol-5-yl)thiazole-4-carboxamide (1-14);
N-(5-(3-((methylamino)methyl)phenyl)-1H-indazol-6-yl)-2-(3-(trifluoromethyl)-1H-pyrazol-5-yl)thiazole-4-carboxamide (1-15);
N-(5-(3-(2-aminopropan-2-yl)phenyl)-1H-indazol-6-yl)-2-(3-(trifluoromethyl)-1H-pyrazol-5-yl)thiazole-4-carboxamide (1-16);
N-(5-(3-(morpholinomethyl)phenyl)-1H-indazol-6-yl)-2-(3-(trifluoromethyl)-1H-pyrazol-5-yl)thiazole-4-carboxamide (1-17);
N-(5-(4-(1-aminocyclopropyl)phenyl)-1H-indazol-6-yl)-2-(3-(trifluoromethyl)-1H-pyrazol-5-yl)thiazole-4-carboxamide (1-18);
N-(5-(3-(aminomethyl)-2-fluorophenyl)-1H-indazol-6-yl)-2-(3-(trifluoromethyl)-1H-pyrazol-5-yl)thiazole-4-carboxamide (1-19);
N-(5-(3-((dimethylamino)methyl)phenyl)-1H-indazol-6-yl)-2-(3-(trifluoromethyl)-1H-pyrazol-5-yl)thiazole-4-carboxamide (1-20);
N-(5-(2-methylisoindolin-5-yl)-1H-indazol-6-yl)-2-(3-(trifluoromethyl)-1H-pyrazol-5-yl)thiazole-4-carboxamide (1-21);
N-(5-(3-((4-methylpiperazin-1-yl)methyl)phenyl)-1H-indazol-6-yl)-2-(3-(trifluoromethyl)-1H-pyrazol-5-yl)thiazole-4-carboxamide (1-22);
N-(5-(3-(1-aminocyclopropyl)phenyl)-1H-indazol-6-yl)-2-(3-(trifluoromethyl)-1H-pyrazol-5-yl)thiazole-4-carboxamide (1-23);
N-(5-(3-oxoisoindolin-5-yl)-1H-indazol-6-yl)-2-(3-(trifluoromethyl)-1H-pyrazol-5-yl)thiazole-4-carboxamide (1-24);
N-(5-(1,2,3,4-tetrahydroisoquinolin-7-yl)-1H-indazol-6-yl)-2-(3-(trifluoromethyl)-1H-pyrazol-5-yl)thiazole-4-carboxamide (1-25);
N-(5-(3-(2-aminoethyl)phenyl)-1H-indazol-6-yl)-2-(3-(trifluoromethyl)-1H-pyrazol-5-yl)thiazole-4-carboxamide (1-26);
N-(5-(2-amino-2,3-dihydro-1H-inden-5-yl)-1H-indazol-6-yl)-2-(3-(trifluoromethyl)-1H-pyrazol-5-yl)thiazole-4-carboxamide (1-27);
N-(5-(3-(pyrrolidin-2-yl)phenyl)-1H-indazol-6-yl)-2-(3-(trifluoromethyl)-1H-pyrazol-5-yl)thiazole-4-carboxamide (1-28);
N-(4-fluoro-5-(isoindolin-5-yl)-1H-indazol-6-yl)-2-(3-(trifluoromethyl)-1H-pyrazol-5-yl)thiazole-4-carboxamide (1-29);
N-(5-(isoindolin-5-yl)-1H-indazol-6-yl)-2-(1H-pyrazol-4-yl)thiazole-4-carboxamide (2-3);
N-(5-(4-(aminomethyl)phenyl)-1H-indazol-6-yl)-2-(1H-pyrazol-4-yl)thiazole-4-carboxamide (2-5);
N-(5-(3-(aminomethyl)phenyl)-1H-indazol-6-yl)-2-(1H-pyrazol-4-yl)thiazole-4-carboxamide (2-6);
N-(5-(3-(piperidin-1-ylmethyl)phenyl)-1H-indazol-6-yl)-2-(3-(trifluoromethyl)-1H-pyrazol-5-yl)thiazole-4-carboxamide (3-5);
N-(5-(3-((2-(dimethylamino)ethylamino)methyl)phenyl)-1H-indazol-6-yl)-2-(3-(trifluoromethyl)-1H-pyrazol-5-yl)thiazole-4-carboxamide (3-6);
N-(5-(3-((benzylamino)methyl)phenyl)-1H-indazol-6-yl)-2-(3-(trifluoromethyl)-1H-pyrazol-5-yl)thiazole-4-carboxamide (3-7);
N-(5-(3-((cyclopropylamino)methyl)phenyl)-1H-indazol-6-yl)-2-(3-(trifluoromethyl)-1H-pyrazol-5-yl)thiazole-4-carboxamide (3-8);
N-(5-(3-((2-methoxyethylamino)methyl)phenyl)-1H-indazol-6-yl)-2-(3-(trifluoromethyl)-1H-pyrazol-5-yl)thiazole-4-carboxamide (3-9);
N-(5-(3-((3-aminopyrrolidin-1-yl)methyl)phenyl)-1H-indazol-6-yl)-2-(3-(trifluoromethyl)-1H-pyrazol-5-yl)thiazole-4-carboxamide (3-10);
N-(5-(3-((3-hydroxypyrrolidin-1-yl)methyl)phenyl)-1H-indazol-6-yl)-2-(3-(trifluoromethyl)-1H-pyrazol-5-yl)thiazole-4-carboxamide (3-11);
N-(5-(3-((2-aminoethylamino)methyl)phenyl)-1H-indazol-6-yl)-2-(3-(trifluoromethyl)-1H-pyrazol-5-yl)thiazole-4-carboxamide (3-12);
N-(5-(3-((2-(methylamino)ethylamino)methyl)phenyl)-1H-indazol-6-yl)-2-(3-(trifluoromethyl)-1H-pyrazol-5-yl)thiazole-4-carboxamide (3-13);
N-(5-(3-((2-hydroxyethylamino)methyl)phenyl)-1H-indazol-6-yl)-2-(3-(trifluoromethyl)-1H-pyrazol-5-yl)thiazole-4-carboxamide (3-14);
N-(5-(6-(aminomethyl)pyridin-3-yl)-1H-indazol-6-yl)-2-(3-(trifluoromethyl)-1H-pyrazol-5-yl)thiazole-4-carboxamide (4-4);
N-(5-(4-(aminomethyl)-2-methylphenyl)-1H-indazol-6-yl)-2-(1H-pyrazol-4-yl)thiazole-4-carboxamide (4-5);
N-(5-(4-(aminomethyl)-2-methylphenyl)-1H-indazol-6-yl)-2-(3-(trifluoromethyl)-1H-pyrazol-5-yl)thiazole-4-carboxamide (4-6);
N-(5-(3-(aminomethyl)-4-fluorophenyl)-1H-indazol-6-yl)-2-(3-(trifluoromethyl)-1H-pyrazol-5-yl)thiazole-4-carboxamide (4-7);
N-(5-(3-(aminomethyl)phenyl)-1H-indazol-6-yl)-2-(3-phenyl-1H-pyrazol-5-yl)thiazole-4-carboxamide (5-6);
N-(5-(isoindolin-5-yl)-1H-indazol-6-yl)-2-(3-methyl-1H-pyrazol-5-yl)thiazole-4-carboxamide (5-7);
N-(5-(3-(aminomethyl)phenyl)-1H-indazol-6-yl)-2-(1-benzyl-1H-pyrazol-4-yl)thiazole-4-carboxamide hydrochloride (8-5);
N-(2-(2-(aminomethyl)thiazol-4-yl)-4-fluorophenyl)-2-(1H-pyrazol-4-yl)thiazole-4-carboxamide hydrochloride (10-6);
2-(cyclopropylethynyl)-N-(5-(isoindolin-5-yl)-1H-indazol-6-yl)thiazole-4-carboxamide hydrochloride (13-5);
N-(5-(3-(aminomethyl)-2-fluorophenyl)-1H-indazol-6-yl)-2-(3-methyl-1H-pyrazol-5-yl)thiazole-4-carboxamide hydrochloride (15-3);
N-(4′-(aminomethyl)-5-fluorobiphenyl-2-yl)-2-(1H-pyrazol-4-yl)thiazole-4-carboxamide (18-4); and
N-(2-(4-(aminomethyl)cyclohex-1-enyl)-4-fluorophenyl)-2-(1H-pyrazol-4-yl)thiazole-4-carboxamide (19-9);
or a pharmaceutically acceptable salt or a stereoisomer thereof.

7. A pharmaceutical composition comprising a pharmaceutical carrier, and dispersed therein, a therapeutically effective amount of a compound of claim 1.

8. The use of the compound according to claim 1 for the preparation of a medicament useful in the treatment or prevention of cancer in a mammal in need of such treatment.


SEQUENCE LISTING (2 sequences)

SEQ ID NO: 1 - protein, 14 aa, Artificial Sequence
  Completely Synthetic Amino Acid Sequence
PLSRTLSVSSLPGL

SEQ ID NO: 2 - protein, 25 aa, Artificial Sequence
  Completely Synthetic Amino Acid Sequence
RREPRILSEEEQEMFRDFDYIADWC